(19) United States
(12) Patent Application Publication
(10) Pub. No.: US 2023/0357335 A1
(43) Pub. Date: Nov. 9, 2023

(54) LOW MOLECULAR WEIGHT PROTEIN COMPOSITIONS

(71) Applicant: Advance International Inc., Livermore, CA (US)

(72) Inventors: Khalid MENTAK, San Ramon, CA (US); Fred JEWETT, Livermore, CA (US); Shahmard Maziar GHORBANI, Alamo, CA (US)

(21) Appl. No.: 17/871,274

(22) Filed: Jul. 22, 2022

Related U.S. Application Data
(63) Continuation of application No. PCT/US2021/014472, filed on Jan. 21, 2021.
(60) Provisional application No. 62/964,606, filed on Jan. 22, 2020.

(51) Int. Cl. C07K 14/46

(52) U.S. Cl. CPC C07K 14/461; A23J 1/04

(57) ABSTRACT

Described herein are protein compositions comprising low molecular weight proteins, water insoluble, and non-hygroscopic properties, methods of treating diseases or conditions using such compositions, and methods of inhibiting angiotensin-1-converting enzyme (ACE) using the same.

### CROSS-REFERENCE

This application is a continuation of International Application No. PCT/US2021/014472, filed Jan. 21, 2021, which claims the benefit of U.S. Provisional Application No. 62/964,606 filed on Jan. 22, 2020, both of which are incorporated herein by reference in their entirety for all purposes.

### BACKGROUND OF THE INVENTION

Bioactive proteins and peptides are conventionally produced by hydrolyzing animal products. These processes include breaking down of high molecular weight proteins either chemically (using acids or bases) or biologically (using enzymes) into small molecular protein units know as peptides. Hydrolyzation of proteins has major drawbacks including the denaturation of proteins, addition of harmful chemicals, and prohibitive manufacturing costs.

### SUMMARY OF THE INVENTION

Described herein are compositions comprising low molecular weight proteins, methods of treating diseases or conditions using such compositions, and methods of inhibiting angiotensin-1-converting enzyme (ACE) using the same.

In one aspect, described herein is a composition comprising an animal protein population, wherein greater than 30% of proteins in said animal protein population have a molecular weight of less than 6000 Daltons.

In some embodiments, the composition comprises greater than 50% of proteins in said animal protein population have a molecular weight of less than 6000 Daltons. In some embodiments, the composition comprises greater than 60% of proteins in said animal protein population have a molecular weight of less than 6000 Daltons.

In another aspect, described herein is a composition comprising a protein population, wherein greater than 30% of proteins in said animal protein population have from 50 to 60 amino acids.

In some embodiments, the composition comprises greater than 50% of proteins in said animal protein population have from 50 to 60 amino acids. In some embodiments, the composition comprises greater than 90% of proteins in said animal protein population have from 50 to 60 amino acids.

In another aspect, described herein is a method of treating a disease or a condition, said method comprising administering to a subject said composition of any one of the preceding claims.

In some embodiments, the disease or condition is hypertension, cardiovascular disease, diabetes, cancer, or Alzheimer's disease. In some embodiments, the disease or condition is malnourishment or an associated condition.

In another aspect, described herein is a method of inhibiting angiotensin-1-converting enzyme (ACE) in a subject, the method comprising administering to a subject in need thereof a therapeutically effective amount of a composition described herein.

Aspects of the disclosure herein provide a composition comprising a protein population, wherein greater than 30% of proteins in the protein population comprise: (a) aldehydes; (b) amides; (c) triazenes; (d) carboxylic acids; (e) alkanes or any combination thereof. In some embodiments, greater than 50% of proteins in said protein population have a molecular weight of less than 6000 Daltons. In some embodiments, greater than 60% of proteins in said protein population have a molecular weight of less than 6000 Daltons.

Aspects of the disclosure herein provide a composition comprising a protein population, wherein greater than 30% of proteins in said population have a molecular weight of less than 6000 Daltons. In some embodiments, greater than 50% of proteins in said protein population have a molecular weight of less than 6000 Daltons. In some embodiments, greater than 60% of proteins in said protein population have a molecular weight of less than 6000 Daltons. In some embodiments, said protein population is water insoluble. In some embodiments, said protein population is non-hygroscopic. In some embodiments, a majority of the proteins in said protein population are non-hydrolyzed proteins. In some embodiments, greater than 90% of proteins in said protein population are non-hydrolyzed proteins. In some embodiments, said protein population comprises one or more proteins with an aldehyde group. In some embodiments, at least 10% of proteins in said protein population comprise an aldehyde group. In some embodiments, at least 50% of proteins in said protein population comprise an aldehyde group. In some embodiments, said protein population comprises one or more proteins with a triazene group. In some embodiments, at least 10% of proteins in said protein population comprise a triazene group. In some embodiments, at least 50% of proteins in said protein population comprise a triazene group. In some embodiments, said protein population comprises one or more proteins with a carboxylic acid group. In some embodiments, at least 10% of proteins in said protein population comprise a carboxylic acid group. In some embodiments, at least 50% of proteins in said protein population comprise a carboxylic acid group. In some embodiments, said protein population comprises one or more proteins with an alkene group. In some embodiments, at least 10% of proteins in said protein population comprise an alkene acid group. In some embodiments, at least 50% of proteins in said protein population comprise an alkene acid group. In some embodiments, said alkene group is a trans alkene group. In some embodiments, said protein population comprises one or more proteins with an amide group. In some embodiments, at least 10% of proteins in said protein population comprise an amide group. In some embodiments, at least 50% of proteins in said protein population comprise an amide group. In some embodiments, said protein population comprises one or more proteins with an alkane group. In some embodiments, at least 10% of proteins in said protein population comprise an alkane group. In some embodiments, at least 50% of proteins in said protein population comprise an alkane group. In some embodiments, at least 10% of proteins in said protein population are bioactive proteins. In some embodiments, at least 50% of proteins in said protein population are bioactive proteins. In some embodiments, at least 90% of proteins in said protein population are bioactive proteins. In some embodiments, said protein population is suitable for human consumption. In some embodiments, said protein population has antihypersensitivity, antithrombotic, antioxidant, antimicrobial, antiproliferative, or anticoagulative properties. In some embodiments, said protein population is an angiotensin-converting-enzyme inhibitor. In some embodiments, greater than 30% of proteins in said protein population have from 50 to 60 amino acids. In some embodiments, greater than 50% of proteins in said protein population have from 50 to 60 amino acids. In some embodiments, greater than 90% of proteins in said protein population have from 50 to 60 amino acids. In some embodiments, said protein population is derived from an animal. In some embodiments, said animal is a fish. In some embodiments, said fish comprises bonito, salmon, sardine, tuna, Alaskan pollack, yellowfin sole, shark, mackerel, cichlid animal, yellowstripe trevally, sardinelle, or hoki. In some embodiments, said animal is a chicken. In some embodiments, said animal is a cow, pig, goat, sheep, or any combination thereof.

Aspects of the disclosure further provide a method of treating a disease or a condition, said method comprising administering to a subject said composition. In some embodiments, said disease or condition is hypertension, cardiovascular disease, diabetes, cancer, or Alzheimer's disease. In some embodiments, said disease or condition is malnourishment or an associated condition.

Aspects of the disclosure further provide a method of inhibiting angiotensin-1-converting enzyme (ACE) in a subject, said method comprising administering to a subject said composition. In some embodiments, said subject is a human.

### INCORPORATION BY REFERENCE

All publications, patents, and patent applications, and NCBI accession numbers mentioned in this specification are herein incorporated by reference to the same extent as if each individual publication, patent, patent application, or NCBI accession number was specifically and individually indicated to be incorporated by reference. To the extent publications and patents, patent applications, or NCBI accession numbers incorporated by reference contradict the disclosure contained in the specification, the specification is intended to supersede and/or take precedence over any such contradictory material. Each of U.S. Pat. Nos. 9,826,757 and 10,039,299 is incorporated herein by reference in its entirety respectively.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1 is an illustration of the protein composition recovery system provided herein showing the various protein composition isolation options in accordance with exemplary embodiments of the present invention. As shown, the protein composition recovery system 1000 includes a grinding unit 100, a slurry preparation tank 150, a dewatering device 170, a protein separation system 10 that can include one or more of a belt filtration system, a rotary drum filter, an immersion extractor, a percolation extractor, a screw press, a decanter centrifuge. or any combination thereof. The product recovery system 1000 also includes a solvent/liquid recovery (SLR) system 700 for recovering the solvent for reuse and for processing the recovered omega-3 oil. The SLR system 700 includes a liquid phase processing unit 705 and a separation unit 715 and optionally a recovered solvent tank 740. The protein composition recovery system 1000 also includes a solvent supply tank 180, discharge stage 30, a dryer unit 800, a vapor condenser 805 and a milling unit 815.

FIG. 2 is a detailed view of a belt filtration system option that can be used as a protein recovery system in accordance with exemplary embodiments of the protein composition recovery system provided herein.

FIG. 3 a detailed view of a rotary drum filtration system option that can be used in accordance with exemplary embodiments of the protein composition recovery system provided herein.

FIG. 4 is a detailed view of an immersion extraction system option that can be used in accordance with exemplary embodiments of the protein composition recovery system provided herein.

FIG. 5 a detailed view of a percolation extraction system option that can be used in accordance with exemplary embodiments of the protein composition recovery system provided herein.

FIG. 6A is a schematic top view and FIG. 6B is a schematic side view of a screw press system option that can be used in accordance with exemplary embodiments of the product recovery system provided herein.

FIG. 7 shows the total spectra absorptions of protein compositions described herein and other known protein compositions.

FIGS. 8A-8B show a comparison of spectral absorption of the protein compositions described herein and a known protein composition derived from animal. FIG. 8A shows the presence of aldehyde groups in the protein compositions described herein while aldehyde groups are absent in known protein composition derived from animal. FIG. 8B shows the presence of amide groups in the protein compositions described herein while amide groups are absent in known protein composition derived from animal.

FIGS. 9A-9C show a comparison of spectral absorption of the protein compositions described herein and a known protein composition derived from peas. FIG. 9A shows the presence of triazene groups in the protein compositions described herein while triazene are absent in the known protein composition derived from peas. FIG. 9B shows the presence of carboxylic acid groups in the protein compositions described herein while carboxylic groups are absent in the known protein composition derived from peas. FIG. 9C shows the presence of alkane groups in the protein compositions described herein while alkane groups are absent in the known protein composition derived from peas.

FIGS. 10A-10C show a comparison of spectral absorption of the protein compositions described herein and a known protein composition derived from whey. FIG. 10A shows the presence of aldehyde groups in the protein compositions described herein while aldehyde groups are absent in the known protein composition derived from whey. FIG. 10B shows the presence of amide groups in the protein compositions described herein while amide groups are absent in the known protein composition derived from whey. FIG. 10C shows the presence of alkene groups in the protein compositions described herein while alkene groups are absent in the known protein composition derived from whey.

### DETAILED DESCRIPTION OF THE INVENTION

While various embodiments of the disclosure have been shown and described herein, it will be obvious to those skilled in the art that such embodiments are provided by way of example only. Numerous variations, changes, and substitutions may occur to those skilled in the art without departing from the disclosure. It should be understood that various alternatives to the embodiments of the disclosure described herein may be employed.

Unless defined otherwise, all technical and scientific terms used herein have the same meaning as commonly understood by one of ordinary skill in the art to which the present disclosure belongs. In case of conflict, the present application including the definitions will control. Also, unless otherwise required by context, singular terms shall include pluralities and plural terms shall include the singular.

### Overview

Bioactive proteins have excellent nutritional value and physiological functions including antihypertensive, antithrombotic, antioxidant, antimicrobial, antiproliferative, anticoagulant effects and act as ACE (angiotensin-1-converting enzyme) inhibitors. Bioactive proteins and peptides are conventionally produced by hydrolyzing animal meat products (Cited in Shahidi et al. “Bioactives from Marine Resources” American Chemical Society pp 24-34, 2008, and Ryan et al. “Bioactive Peptides from Muscle Source: Meat and Animal.” Nutrients. 3: 765-791, 2011). Bioactive peptides from Pacific Hake animal, shark and Alaska Pollock have been reported to contain ACE inhibitory peptide (peptides that inhibit the body enzyme responsible for high blood pressure) and antioxidant activity, respectively (Cited in Shahidi et al., 2008 and Ryan et al., 2011). However, conventional bioactive proteins and peptides are produced by breaking down (i.e. hydrolyzing) high molecular weight proteins either chemically (using acids or bases) or biologically (using enzymes) into small molecular protein units know as peptides. Hydrolyzation has major drawbacks including the denaturation of proteins, addition of harmful chemicals, and prohibitive manufacturing costs.

It was shown that the higher the percentage of smaller molecular peptide or polypeptide fractions in a protein solution the greater is the bioactivity and the faster is the digestion in the body. The food and pharmaceutical industries have strong demands for low molecular weight bioactive proteins based on the major benefits afforded by this class of proteins in contrast with conventional proteins. However, currently, there are no non-hydrolyzed proteins with a molecular weight profile that supports fast digestion and significant bioactivity. In fact, most hydrolyzed proteins still retain a significant population of large molecular weight peptides and have lower digestibility and very limited bioactivity. It is hypothesized that a protein with a large population of species with low molecular weight would provide a unique combination of fast digestibility for optimal nutrition and enhanced bioactivity for therapeutic effects. Accordingly, in one aspect, described herein is a protein composition comprising a protein population, wherein greater than 30% of proteins in said protein population have a low molecular weight. In some embodiments, greater than 30% of proteins in the protein population have a molecular weight of less than 6000 Daltons. In some embodiments, about 69% of proteins in the protein population have a molecular weight of 5000 Daltons or less. In some embodiments a protein in the protein population is derived from an animal protein.

Additionally, a protein composition with a highly concentrated (percent weight or “wt %”) protein composition provides a high nutrition value per gram of protein composition consumed. One distinctive structural feature of a protein composition indicative of a high concentration of protein is the presence of amides. A higher concentration of amides indicates a higher concentration of protein. Accordingly, in one aspect, described herein is a protein composition comprising from about 10 wt % to about 95 wt % of protein.

A protein composition with a complete amino acid profile (a Protein digestibility-corrected amino acid score “PDCAAS” score of 1 or greater) provides optimal nutritional value as a standalone product or as an added ingredient. Accordingly, in one aspect, described herein is a protein composition comprising a PDCAAS score in the range of from about 0.6 to about 1.0.

A protein composition that is water insoluble and non-hygroscopic provides an enormous advantage in food processing since water soluble proteins because uncontrolled viscosity increases, and a host of problems associated with adhesion to equipment and even damage to presses and extruders. Generally, the potential for introducing novel protein-enriched food products conventionally limited by protein's natural water solubility. Thus, water insolubility allows for vastly improved versatility in food preparations. Additionally, a protein composition (e.g., protein powder) with a hygroscopic property (binds to water from the environment) is prone to clumping, spoilage and hazardous work conditions costing manufacturers added expenses. One distinctive structural feature of a protein composition indicative of water insolubility and non-hygroscopicity is the presence of alkenes, alkanes, and aldehydes. A higher concentration of alkenes, alkanes, and aldehydes in a protein composition provides for a higher degree of water insolubility and non-hygroscopicity. Accordingly, in one aspect, described herein is a protein composition that is water insoluble. In some embodiments, the protein composition does not form a viscous solution when combined to aqueous solutions (e.g. water). In some embodiments, the protein composition forms smooth dispersions in variety of hydrophilic and hydrophobic liquids. In some embodiments, the protein composition provides flexibility in formulating existing and novel foods that is not disclosed in the art. In some embodiments, the protein composition is non-hygroscopic. In some embodiments, the protein composition provides an ability to save costs on storage and handling during protein application processing.

### Protein Compositions

In some embodiments, the protein composition described herein comprises greater than 20% of proteins in said animal protein population having a molecular weight of less than 6000 Daltons. In some embodiments, the composition comprises greater than 25% of proteins in said animal protein population having a molecular weight of less than 6000 Daltons. In some embodiments, the composition comprises greater than 30% of proteins in said animal protein population having a molecular weight of less than 6000 Daltons. In some embodiments, the composition comprises greater than 35% of proteins in said animal protein population having a molecular weight of less than 6000 Daltons. In some embodiments, the composition comprises greater than 40% of proteins in said animal protein population having a molecular weight of less than 6000 Daltons. In some embodiments, the composition comprises greater than 45% of proteins in said animal protein population having a molecular weight of less than 6000 Daltons. In some embodiments, the composition comprises greater than 50% of proteins in said animal protein population having a molecular weight of less than 6000 Daltons. In some embodiments, the composition comprises greater than 55% of proteins in said animal protein population having a molecular weight of less than 6000 Daltons. In some embodiments, the composition comprises greater than 60% of proteins in said animal protein population having a molecular weight of less than 6000 Daltons. In some embodiments, the composition comprises greater than 65% of proteins in said animal protein population having a molecular weight of less than 6000 Daltons. In some embodiments, the composition comprises greater than 70% of proteins in said animal protein population having a molecular weight of less than 6000 Daltons. In some embodiments, the composition comprises greater than 75% of proteins in said animal protein population having a molecular weight of less than 6000 Daltons. In some embodiments, the composition comprises greater than 80% of proteins in said animal protein population having a molecular weight of less than 6000 Daltons. In some embodiments, the composition comprises greater than 85% of proteins in said animal protein population having a molecular weight of less than 6000 Daltons. In some embodiments, the composition comprises greater than 90% of proteins in said animal protein population having a molecular weight of less than 6000 Daltons. In some embodiments, about 69% of proteins in the protein population have a molecular weight of less than 6000 Daltons.

In some embodiments, the protein composition described herein comprises greater than 20% of proteins in said animal protein population having a molecular weight from 5800 to 6000 Daltons. In some embodiments, the composition comprises greater than 25% of proteins in said animal protein population having a molecular weight from 5800 to 6000 Daltons. In some embodiments, the composition comprises greater than 30% of proteins in said animal protein population having a molecular weight from 5800 to 6000 Daltons. In some embodiments, the composition comprises greater than 35% of proteins in said animal protein population having a molecular weight from 5800 to 6000 Daltons. In some embodiments, the composition comprises greater than 40% of proteins in said animal protein population having a molecular weight from 5800 to 6000 Daltons. In some embodiments, the composition comprises greater than 45% of proteins in said animal protein population having a molecular weight from 5800 to 6000 Daltons. In some embodiments, the composition comprises greater than 50% of proteins in said animal protein population having a molecular weight from 5800 to 6000 Daltons. In some embodiments, the composition comprises greater than 55% of proteins in said animal protein population having a molecular weight from 5800 to 6000 Daltons. In some embodiments, the composition comprises greater than 60% of proteins in said animal protein population having a molecular weight from 5800 to 6000 Daltons. In some embodiments, the composition comprises greater than 65% of proteins in said animal protein population having a molecular weight from 5800 to 6000 Daltons. In some embodiments, the composition comprises greater than 70% of proteins in said animal protein population having a molecular weight from 5800 to 6000 Daltons. In some embodiments, the composition comprises greater than 75% of proteins in said animal protein population having a molecular weight from 5800 to 6000 Daltons. In some embodiments, the composition comprises greater than 80% of proteins in said animal protein population having a molecular weight from 5800 to 6000 Daltons. In some embodiments, the composition comprises greater than 85% of proteins in said animal protein population having a molecular weight from 5800 to 6000 Daltons. In some embodiments, the composition comprises greater than 90% of proteins in said animal protein population having a molecular weight from 5800 to 6000 Daltons. In some embodiments, about 69% of proteins in the protein population have a molecular weight from 5800 to 6000 Daltons.

In some embodiments, the protein composition described herein comprises greater than 20% of proteins in said animal protein population having a molecular weight from 5500 to 6000 Daltons. In some embodiments, the composition comprises greater than 25% of proteins in said animal protein population having a molecular weight from 5500 to 6000 Daltons. In some embodiments, the composition comprises greater than 30% of proteins in said animal protein population having a molecular weight from 5500 to 6000 Daltons. In some embodiments, the composition comprises greater than 35% of proteins in said animal protein population having a molecular weight from 5500 to 6000 Daltons. In some embodiments, the composition comprises greater than 40% of proteins in said animal protein population having a molecular weight from 5500 to 6000 Daltons. In some embodiments, the composition comprises greater than 45% of proteins in said animal protein population having a molecular weight from 5500 to 6000 Daltons. In some embodiments, the composition comprises greater than 50% of proteins in said animal protein population having a molecular weight from 5500 to 6000 Daltons. In some embodiments, the composition comprises greater than 55% of proteins in said animal protein population having a molecular weight from 5500 to 6000 Daltons. In some embodiments, the composition comprises greater than 60% of proteins in said animal protein population having a molecular weight from 5500 to 6000 Daltons. In some embodiments, the composition comprises greater than 65% of proteins in said animal protein population having a molecular weight from 5500 to 6000 Daltons. In some embodiments, the composition comprises greater than 70% of proteins in said animal protein population having a molecular weight from 5500 to 6000 Daltons. In some embodiments, the composition comprises greater than 75% of proteins in said animal protein population having a molecular weight from 5500 to 6000 Daltons. In some embodiments, the composition comprises greater than 80% of proteins in said animal protein population having a molecular weight from 5500 to 6000 Daltons. In some embodiments, the composition comprises greater than 85% of proteins in said animal protein population having a molecular weight from 5500 to 6000 Daltons. In some embodiments, the composition comprises greater than 90% of proteins in said animal protein population having a molecular weight from 5500 to 6000 Daltons. In some embodiments, about 69% of proteins in the protein population have a molecular weight from 5500 to 6000 Daltons.

In some embodiments, the protein composition described herein comprises greater than 20% of proteins in said animal protein population having a molecular weight from 5000 to 6000 Daltons. In some embodiments, the composition comprises greater than 25% of proteins in said animal protein population having a molecular weight from 5000 to 6000 Daltons. In some embodiments, the composition comprises greater than 30% of proteins in said animal protein population having a molecular weight from 5000 to 6000 Daltons. In some embodiments, the composition comprises greater than 35% of proteins in said animal protein population having a molecular weight from 5000 to 6000 Daltons. In some embodiments, the composition comprises greater than 40% of proteins in said animal protein population having a molecular weight from 5000 to 6000 Daltons. In some embodiments, the composition comprises greater than 45% of proteins in said animal protein population having a molecular weight from 5000 to 6000 Daltons. In some embodiments, the composition comprises greater than 50% of proteins in said animal protein population having a molecular weight from 5000 to 6000 Daltons. In some embodiments, the composition comprises greater than 55% of proteins in said animal protein population having a molecular weight from 5000 to 6000 Daltons. In some embodiments, the composition comprises greater than 60% of proteins in said animal protein population having a molecular weight from 5000 to 6000 Daltons. In some embodiments, the composition comprises greater than 65% of proteins in said animal protein population having a molecular weight from 5000 to 6000 Daltons. In some embodiments, the composition comprises greater than 70% of proteins in said animal protein population having a molecular weight from 5000 to 6000 Daltons. In some embodiments, the composition comprises greater than 75% of proteins in said animal protein population having a molecular weight from 5000 to 6000 Daltons. In some embodiments, the composition comprises greater than 80% of proteins in said animal protein population having a molecular weight from 5000 to 6000 Daltons. In some embodiments, the composition comprises greater than 85% of proteins in said animal protein population having a molecular weight from 5000 to 6000 Daltons. In some embodiments, the composition comprises greater than 90% of proteins in said animal protein population having a molecular weight from 5000 to 6000 Daltons. In some embodiments, about 69% of proteins in the protein population have a molecular weight from 5000 to 6000 Daltons.

In some embodiments, the protein composition described herein comprises greater than 20% of proteins in said animal protein population having a molecular weight equal to or less than 5000 Daltons. In some embodiments, the composition comprises greater than 25% of proteins in said animal protein population having a molecular weight equal to or less than 5000 Daltons. In some embodiments, the composition comprises greater than 30% of proteins in said animal protein population having a molecular weight equal to or less than 5000 Daltons. In some embodiments, the composition comprises greater than 35% of proteins in said animal protein population having a molecular weight equal to or less than 5000 Daltons. In some embodiments, the composition comprises greater than 40% of proteins in said animal protein population having a molecular weight equal to or less than 5000 Daltons. In some embodiments, the composition comprises greater than 45% of proteins in said animal protein population having a molecular weight equal to or less than 5000 Daltons. In some embodiments, the composition comprises greater than 50% of proteins in said animal protein population having a molecular weight equal to or less than 5000 Daltons. In some embodiments, the composition comprises greater than 55% of proteins in said animal protein population having a molecular weight equal to or less than 5000 Daltons. In some embodiments, the composition comprises greater than 60% of proteins in said animal protein population having a molecular weight equal to or less than 5000 Daltons. In some embodiments, the composition comprises greater than 65% of proteins in said animal protein population having a molecular weight equal to or less than 5000 Daltons. In some embodiments, the composition comprises greater than 70% of proteins in said animal protein population having a molecular weight equal to or less than 5000 Daltons. In some embodiments, the composition comprises greater than 75% of proteins in said animal protein population having a molecular weight equal to or less than 5000 Daltons. In some embodiments, the composition comprises greater than 80% of proteins in said animal protein population having a molecular weight equal to or less than 5000 Daltons. In some embodiments, the composition comprises greater than 85% of proteins in said animal protein population having a molecular weight equal to or less than 5000 Daltons. In some embodiments, the composition comprises greater than 90% of proteins in said animal protein population having a molecular weight equal to or less than 5000 Daltons. In some embodiments, about 69% of proteins in the protein population have a molecular weight equal to or less than 5000 Daltons.

In some embodiments, the protein composition described herein comprises greater than 20% of proteins in said animal protein population having a molecular weight of more than 6000 Daltons. In some embodiments, the composition comprises greater than 25% of proteins in said animal protein population having a molecular weight of more than 6000 Daltons. In some embodiments, the composition comprises greater than 30% of proteins in said animal protein population having a molecular weight of more than 6000 Daltons. In some embodiments, the composition comprises greater than 35% of proteins in said animal protein population having a molecular weight of more than 6000 Daltons. In some embodiments, the composition comprises greater than 40% of proteins in said animal protein population having a molecular weight of more than 6000 Daltons. In some embodiments, the composition comprises greater than 45% of proteins in said animal protein population having a molecular weight of more than 6000 Daltons. In some embodiments, the composition comprises greater than 50% of proteins in said animal protein population having a molecular weight of more than 6000 Daltons. In some embodiments, the composition comprises greater than 55% of proteins in said animal protein population having a molecular weight of more than 6000 Daltons. In some embodiments, the composition comprises greater than 60% of proteins in said animal protein population having a molecular weight of more than 6000 Daltons. In some embodiments, the composition comprises greater than 65% of proteins in said animal protein population having a molecular weight of more than 6000 Daltons. In some embodiments, the composition comprises greater than 70% of proteins in said animal protein population having a molecular weight of more than 6000 Daltons. In some embodiments, the composition comprises greater than 75% of proteins in said animal protein population having a molecular weight of more than 6000 Daltons. In some embodiments, the composition comprises greater than 80% of proteins in said animal protein population having a molecular weight of more than 6000 Daltons. In some embodiments, the composition comprises greater than 85% of proteins in said animal protein population having a molecular weight of more than 6000 Daltons. In some embodiments, the composition comprises greater than 90% of proteins in said animal protein population having a molecular weight of more than 6000 Daltons. In some embodiments, about 69% of proteins in the protein population have a molecular weight of more than 6000 Daltons.

In some embodiments, the protein composition described herein comprises greater than 20% of proteins in said animal protein population having a molecular weight from 5800 to 6000 Daltons. In some embodiments, the composition comprises greater than 25% of proteins in said animal protein population having a molecular weight from 5800 to 6000 Daltons. In some embodiments, the composition comprises greater than 30% of proteins in said animal protein population having a molecular weight from 5800 to 6000 Daltons. In some embodiments, the composition comprises greater than 35% of proteins in said animal protein population having a molecular weight from 5800 to 6000 Daltons. In some embodiments, the composition comprises greater than 40% of proteins in said animal protein population having a molecular weight from 5800 to 6000 Daltons. In some embodiments, the composition comprises greater than 45% of proteins in said animal protein population having a molecular weight from 5800 to 6000 Daltons. In some embodiments, the composition comprises greater than 50% of proteins in said animal protein population having a molecular weight from 5800 to 6000 Daltons. In some embodiments, the composition comprises greater than 55% of proteins in said animal protein population having a molecular weight from 5800 to 6000 Daltons. In some embodiments, the composition comprises greater than 60% of proteins in said animal protein population having a molecular weight from 5800 to 6000 Daltons. In some embodiments, the composition comprises greater than 65% of proteins in said animal protein population having a molecular weight from 5800 to 6000 Daltons. In some embodiments, the composition comprises greater than 70% of proteins in said animal protein population having a molecular weight from 5800 to 6000 Daltons. In some embodiments, the composition comprises greater than 75% of proteins in said animal protein population having a molecular weight from 5800 to 6000 Daltons. In some embodiments, the composition comprises greater than 80% of proteins in said animal protein population having a molecular weight from 5800 to 6000 Daltons. In some embodiments, the composition comprises greater than 85% of proteins in said animal protein population having a molecular weight from 5800 to 6000 Daltons. In some embodiments, the composition comprises greater than 90% of proteins in said animal protein population having a molecular weight from 5800 to 6000 Daltons. In some embodiments, about 69% of proteins in the protein population have a molecular weight from 5800 to 6000 Daltons.

In some embodiments, the protein composition described herein comprises greater than 20% of proteins in said animal protein population having a molecular weight from 5500 to 6000 Daltons. In some embodiments, the composition comprises greater than 25% of proteins in said animal protein population having a molecular weight from 5500 to 6000 Daltons. In some embodiments, the composition comprises greater than 30% of proteins in said animal protein population having a molecular weight from 5500 to 6000 Daltons. In some embodiments, the composition comprises greater than 35% of proteins in said animal protein population having a molecular weight from 5500 to 6000 Daltons. In some embodiments, the composition comprises greater than 40% of proteins in said animal protein population having a molecular weight from 5500 to 6000 Daltons. In some embodiments, the composition comprises greater than 45% of proteins in said animal protein population having a molecular weight from 5500 to 6000 Daltons. In some embodiments, the composition comprises greater than 50% of proteins in said animal protein population having a molecular weight from 5500 to 6000 Daltons. In some embodiments, the composition comprises greater than 55% of proteins in said animal protein population having a molecular weight from 5500 to 6000 Daltons. In some embodiments, the composition comprises greater than 60% of proteins in said animal protein population having a molecular weight from 5500 to 6000 Daltons. In some embodiments, the composition comprises greater than 65% of proteins in said animal protein population having a molecular weight from 5500 to 6000 Daltons. In some embodiments, the composition comprises greater than 70% of proteins in said animal protein population having a molecular weight from 5500 to 6000 Daltons. In some embodiments, the composition comprises greater than 75% of proteins in said animal protein population having a molecular weight from 5500 to 6000 Daltons. In some embodiments, the composition comprises greater than 80% of proteins in said animal protein population having a molecular weight from 5500 to 6000 Daltons. In some embodiments, the composition comprises greater than 85% of proteins in said animal protein population having a molecular weight from 5500 to 6000 Daltons. In some embodiments, the composition comprises greater than 90% of proteins in said animal protein population having a molecular weight from 5500 to 6000 Daltons. In some embodiments, about 69% of proteins in the protein population have a molecular weight from 5500 to 6000 Daltons.

In some embodiments, the protein composition described herein comprises greater than 20% of proteins in said animal protein population having a molecular weight from 5000 to 6000 Daltons. In some embodiments, the composition comprises greater than 25% of proteins in said animal protein population having a molecular weight from 5000 to 6000 Daltons. In some embodiments, the composition comprises greater than 30% of proteins in said animal protein population having a molecular weight from 5000 to 6000 Daltons. In some embodiments, the composition comprises greater than 35% of proteins in said animal protein population having a molecular weight from 5000 to 6000 Daltons. In some embodiments, the composition comprises greater than 40% of proteins in said animal protein population having a molecular weight from 5000 to 6000 Daltons. In some embodiments, the composition comprises greater than 45% of proteins in said animal protein population having a molecular weight from 5000 to 6000 Daltons. In some embodiments, the composition comprises greater than 50% of proteins in said animal protein population having a molecular weight from 5000 to 6000 Daltons. In some embodiments, the composition comprises greater than 55% of proteins in said animal protein population having a molecular weight from 5000 to 6000 Daltons. In some embodiments, the composition comprises greater than 60% of proteins in said animal protein population having a molecular weight from 5000 to 6000 Daltons. In some embodiments, the composition comprises greater than 65% of proteins in said animal protein population having a molecular weight from 5000 to 6000 Daltons. In some embodiments, the composition comprises greater than 70% of proteins in said animal protein population having a molecular weight from 5000 to 6000 Daltons. In some embodiments, the composition comprises greater than 75% of proteins in said animal protein population having a molecular weight from 5000 to 6000 Daltons. In some embodiments, the composition comprises greater than 80% of proteins in said animal protein population having a molecular weight from 5000 to 6000 Daltons. In some embodiments, the composition comprises greater than 85% of proteins in said animal protein population having a molecular weight from 5000 to 6000 Daltons. In some embodiments, the composition comprises greater than 90% of proteins in said animal protein population having a molecular weight from 5000 to 6000 Daltons. In some embodiments, about 69% of proteins in the protein population have a molecular weight from 5000 to 6000 Daltons.

In some embodiments, the protein composition described herein comprises greater than 20% of proteins in said animal protein population having a molecular weight equal to or more than 5000 Daltons. In some embodiments, the composition comprises greater than 25% of proteins in said animal protein population having a molecular weight equal to or more than 5000 Daltons. In some embodiments, the composition comprises greater than 30% of proteins in said animal protein population having a molecular weight equal to or more than 5000 Daltons. In some embodiments, the composition comprises greater than 35% of proteins in said animal protein population having a molecular weight equal to or more than 5000 Daltons. In some embodiments, the composition comprises greater than 40% of proteins in said animal protein population having a molecular weight equal to or more than 5000 Daltons. In some embodiments, the composition comprises greater than 45% of proteins in said animal protein population having a molecular weight equal to or more than 5000 Daltons. In some embodiments, the composition comprises greater than 50% of proteins in said animal protein population having a molecular weight equal to or more than 5000 Daltons. In some embodiments, the composition comprises greater than 55% of proteins in said animal protein population having a molecular weight equal to or more than 5000 Daltons. In some embodiments, the composition comprises greater than 60% of proteins in said animal protein population having a molecular weight equal to or more than 5000 Daltons. In some embodiments, the composition comprises greater than 65% of proteins in said animal protein population having a molecular weight equal to or more than 5000 Daltons. In some embodiments, the composition comprises greater than 70% of proteins in said animal protein population having a molecular weight equal to or more than 5000 Daltons. In some embodiments, the composition comprises greater than 75% of proteins in said animal protein population having a molecular weight equal to or more than 5000 Daltons. In some embodiments, the composition comprises greater than 80% of proteins in said animal protein population having a molecular weight equal to or more than 5000 Daltons. In some embodiments, the composition comprises greater than 85% of proteins in said animal protein population having a molecular weight equal to or more than 5000 Daltons. In some embodiments, the composition comprises greater than 90% of proteins in said animal protein population having a molecular weight equal to or more than 5000 Daltons. In some embodiments, about 69% of proteins in the protein population have a molecular weight equal to or more than 5000 Daltons.

In some embodiments, a majority of proteins in said animal protein population are non-hydrolyzed proteins. In some embodiments, greater than 50% of proteins in said animal protein population are non-hydrolyzed proteins. In some embodiments, greater than 60% of proteins in said animal protein population are non-hydrolyzed proteins. In some embodiments, greater than 70% of proteins in said animal protein population are non-hydrolyzed proteins. In some embodiments, greater than 80% of proteins in said animal protein population are non-hydrolyzed proteins. In some embodiments, greater than 90% of proteins in said animal protein population are non-hydrolyzed proteins. In some embodiments, greater than 95% of proteins in said animal protein population are non-hydrolyzed proteins. In some embodiments, the protein used in the protein compositions disclosed herein is non-hydrolyzed. In some examples, the protein in the protein compositions disclosed herein has a degree of hydrolysis of about 1% to about 50%. In some examples, the protein in the protein compositions disclosed herein has a degree of hydrolysis of at least about 1%. In some examples, the protein in the protein compositions disclosed herein has a degree of hydrolysis of at most about 50%. In some examples, the protein in the protein compositions disclosed herein has a degree of hydrolysis of about 1% to about 2%, about 1% to about 4%, about 1% to about 6%, about 1% to about 8%, about 1% to about 10%, about 1% to about 15%, about 1% to about 20%, about 1% to about 25%, about 1% to about 30%, about 1% to about 40%, about 1% to about 50%, about 2% to about 4%, about 2% to about 6%, about 2% to about 8%, about 2% to about 10%, about 2% to about 15%, about 2% to about 20%, about 2% to about 25%, about 2% to about 30%, about 2% to about 40%, about 2% to about 50%, about 4% to about 6%, about 4% to about 8%, about 4% to about 10%, about 4% to about 15%, about 4% to about 20%, about 4% to about 25%, about 4% to about 30%, about 4% to about 40%, about 4% to about 50%, about 6% to about 8%, about 6% to about 10%, about 6% to about 15%, about 6% to about 20%, about 6% to about 25%, about 6% to about 30%, about 6% to about 40%, about 6% to about 50%, about 8% to about 10%, about 8% to about 15%, about 8% to about 20%, about 8% to about 25%, about 8% to about 30%, about 8% to about 40%, about 8% to about 50%, about 10% to about 15%, about 10% to about 20%, about 10% to about 25%, about 10% to about 30%, about 10% to about 40%, about 10% to about 50%, about 15% to about 20%, about 15% to about 25%, about 15% to about 30%, about 15% to about 40%, about 15% to about 50%, about 20% to about 25%, about 20% to about 30%, about 20% to about 40%, about 20% to about 50%, about 25% to about 30%, about 25% to about 40%, about 25% to about 50%, about 30% to about 40%, about 30% to about 50%, or about 40% to about 50%. In some examples, the protein in the protein compositions disclosed herein has a degree of hydrolysis of about 1%, about 2%, about 4%, about 6%, about 8%, about 10%, about 15%, about 20%, about 25%, about 30%, about 40%, or about 50%.

In various embodiments, the protein compositions of the disclosure have low osmolality. In some embodiments, the protein compositions described herein have an osmolality between about 100 mOsm/kg and 1000 mOsm/kg, for example between 200 mOsm/kg and 1000 mOsm/kg, between 300 mOsm/kg and 1000 mOsm/kg, between 400 mOsm/kg and 1000 mOsm/kg, between 500 mOsm/kg and 1000 mOsm/kg, between 600 mOsm/kg and 1000 mOsm/kg, between 700 mOsm/kg and 1000 mOsm/kg, between 800 mOsm/kg and 1000 mOsm/kg, or between 900 mOsm/kg and 1000 mOsm/kg. In some embodiments, the osmolality of the protein compositions described herein is less than about 300 mOsm/kg, less than about 275 mOsm/kg, less than about 250 mOsm/kg, less than about 240 mOsm/kg, less than about 230 mOsm/kg, less than about 220 mOsm/kg, less than about 210 mOsm/kg, or less than about 200 mOsm/kg.

In some embodiments, the protein in the protein compositions has a digestibility coefficient of at least about 70%. In some embodiments, the protein in the protein compositions has a digestibility coefficient of at least about 80%. In some embodiments, the protein in the protein compositions has a digestibility coefficient of at least about 90%. In some embodiments, the protein in the protein compositions has a digestibility coefficient of at least about 95%. In some embodiments, the protein in the protein compositions has a digestibility coefficient of at least about 97%. In some embodiments, the protein in the protein compositions has a digestibility coefficient of at least about 98%. In some embodiments, the protein in the protein compositions has a digestibility coefficient of at least about 99%.

In various embodiments, the protein compositions described herein have a low glycemic index. The glycemic index (GI) is a ranking of food on a scale from 0 to 100 according to the extent to which they raise blood sugar levels after eating. Glucose for example has a GI of 100, sucrose has a GI of 65+ and isomaltulose has a GI of 32. Foods with a high GI (>50) are those which are rapidly digested and absorbed and result in marked fluctuations in blood sugar levels. Low-GI foods (<40), by virtue of their slow digestion and absorption, produce gradual rises in blood sugar and insulin levels, and have proven beneficial for health. Low GI diets have shown to improve both glucose and lipid levels in humans with diabetes (type 1 and type 2). They also have benefits in weight control because they help to control appetite and delay hunger. Low GI diets also reduce insulin levels and insulin resistance. In some embodiments, the protein compositions described herein have a GI of about 0 to about 70. In some embodiments, the protein compositions described herein have a GI of at least about 0. In some embodiments, the protein compositions described herein have a GI of at most about 70. In some embodiments, the protein compositions described herein have a GI of about 0 to about 10, about 0 to about 20, about 0 to about 30, about 0 to about 40, about 0 to about 50, about 0 to about 60, about 0 to about 70, about 10 to about 20, about 10 to about 30, about 10 to about 40, about 10 to about 50, about 10 to about 60, about 10 to about 70, about 20 to about 30, about 20 to about 40, about 20 to about 50, about 20 to about 60, about 20 to about 70, about 30 to about 40, about 30 to about 50, about 30 to about 60, about 30 to about 70, about 40 to about 50, about 40 to about 60, about 40 to about 70, about 50 to about 60, about 50 to about 70, or about 60 to about 70. In some embodiments, the protein compositions described herein have a GI of about 0, about 10, about 20, about 30, about 40, about 50, about 60, or about 70.

As used herein, the term “glycemic load” refers to the impact of a food on blood glucose. Glycemic load refers to the glycemic index of a food relative to its carbohydrate load. Thus, glycemic load is calculated as follows: (Glycemic Index/Available Carbohydrates)×100. In certain embodiments, the protein compositions disclosed herein will have a glycemic load of not more than about 12 on a 50 g basis. In certain embodiments, the protein compositions disclosed herein will have a glycemic load of not more than about 10 on a 50 g reference basis. In certain embodiments, the protein compositions disclosed herein will have a glycemic load of not more than about 8 on a 50 g reference basis. In certain embodiments, the protein compositions disclosed herein will have a glycemic load of not more than about 6 on a 50 g reference basis. In certain embodiments, the protein compositions disclosed herein will have a glycemic load of not more than about 4 on a 50 g reference basis. In certain embodiments, the protein compositions disclosed herein will have a glycemic load of not more than about 2 on a 50 g reference basis.

The protein compositions described herein comprise a protein. The amount of protein in the protein compositions will depend on the targeted application of the protein compositions. In some embodiments, the protein compositions described have a high protein content. In some examples, the protein compositions described herein comprise from about 10 wt % to about 95 wt % of protein. In some examples, the protein compositions described herein comprise from about 15 wt % to about 90 wt % of protein. In some examples, the protein compositions described herein comprise from about 20 wt % to about 80 wt % of protein. In some examples, the amount of protein in the protein compositions described herein is from about 20 wt % to about 75 wt %. In some examples, the amount of protein in the protein compositions described herein is at least about 20 wt %. In some examples, the amount of protein in the protein compositions described herein is from about 20 wt % to about 25 wt %, about 20 wt % to about 30 wt %, about 20 wt % to about 35 wt %, about 20 wt % to about 40 wt %, about 20 wt % to about 45 wt %, about 20 wt % to about 50 wt %, about 20 wt % to about 55 wt %, about 20 wt % to about 60 wt %, about 20 wt % to about 65 wt %, about 20 wt % to about 70 wt %, about 20 wt % to about 75 wt %, about 20 wt % to about 80 wt %, about 20 wt % to about 85 wt %, about 20 wt % to about 90 wt %, about 20 wt % to about 95 wt %, about 25 wt % to about 30 wt %, about 25 wt % to about 35 wt %, about 25 wt % to about 40 wt %, about 25 wt % to about 45 wt %, about 25 wt % to about 50 wt %, about 25 wt % to about 55 wt %, about 25 wt % to about 60 wt %, about 25 wt % to about 65 wt %, about 25 wt % to about 70 wt %, about 25 wt % to about 75 wt %, about 25 wt % to about 80 wt %, about 25 wt % to about 85 wt %, about 25 wt % to about 90 wt %, about 25 wt % to about 95 wt %, about 30 wt % to about 35 wt %, about 30 wt % to about 40 wt %, about 30 wt % to about 45 wt %, about 30 wt % to about 50 wt %, about 30 wt % to about 55 wt %, about 30 wt % to about 60 wt %, about 30 wt % to about 65 wt %, about 30 wt % to about 70 wt %, about 30 wt % to about 75 wt %, about 30 wt % to about 80 wt %, about 30 wt % to about 85 wt %, about 30 wt % to about 90 wt %, about 30 wt % to about 95 wt %, about 35 wt % to about 40 wt %, about 35 wt % to about 45 wt %, about 35 wt % to about 50 wt %, about 35 wt % to about 55 wt %, about 35 wt % to about 60 wt %, about 35 wt % to about 65 wt %, about 35 wt % to about 70 wt %, about 35 wt % to about 75 wt %, about 35 wt % to about 80 wt %, about 35 wt % to about 85 wt %, about 35 wt % to about 90 wt %, about 35 wt % to about 95 wt %, about 40 wt % to about 45 wt %, about 40 wt % to about 50 wt %, about 40 wt % to about 55 wt %, about 40 wt % to about 60 wt %, about 40 wt % to about 65 wt %, about 40 wt % to about 70 wt %, about 40 wt % to about 75 wt %, about 40 wt % to about 80 wt %, about 40 wt % to about 85 wt %, about 40 wt % to about 90 wt %, about 40 wt % to about 95 wt %, about 45 wt % to about 50 wt %, about 45 wt % to about 55 wt %, about 45 wt % to about 60 wt %, about 45 wt % to about 65 wt %, about 45 wt % to about 70 wt %, about 45 wt % to about 75 wt %, about 45 wt % to about 80 wt %, about 45 wt % to about 85 wt %, about 45 wt % to about 90 wt %, about 45 wt % to about 95 wt %, about 50 wt % to about 55 wt %, about 50 wt % to about 60 wt %, about 50 wt % to about 65 wt %, about 50 wt % to about 70 wt %, about 50 wt % to about 75 wt %, about 50 wt % to about 80 wt %, about 50 wt % to about 85 wt %, about 50 wt % to about 90 wt %, about 50 wt % to about 95 wt %, about 55 wt % to about 60 wt %, about 55 wt % to about 65 wt %, about 55 wt % to about 70 wt %, about 55 wt % to about 75 wt %, about 55 wt % to about 80 wt %, about 55 wt % to about 85 wt %, about 55 wt % to about 90 wt %, about 55 wt % to about 95 wt %, about 60 wt % to about 65 wt %, about 60 wt % to about 70 wt %, about 60 wt % to about 75 wt %, about 60 wt % to about 80 wt %, about 60 wt % to about 85 wt %, about 60 wt % to about 90 wt %, about 60 wt % to about 95 wt %, about 65 wt % to about 70 wt %, about 65 wt % to about 75 wt %, about 65 wt % to about 80 wt %, about 65 wt % to about 85 wt %, about 65 wt % to about 90 wt %, about 65 wt % to about 95 wt %, about 70 wt % to about 75 wt %, about 70 wt % to about 80 wt %, about 70 wt % to about 85 wt %, about 70 wt % to about 90 wt %, about 70 wt % to about 95 wt %, about 75 wt % to about 80 wt %, about 75 wt % to about 85 wt %, about 75 wt % to about 90 wt %, about 75 wt % to about 95 wt %, about 80 wt % to about 85 wt %, about 80 wt % to about 90 wt %, about 80 wt % to about 95 wt %, about 85 wt % to about 90 wt %, about 85 wt % to about 95 wt %, or about 90 wt % to about 95 wt %. In some examples, the amount of protein in the protein compositions described herein is from about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, about 90 wt %, or about 95 wt %.

In some embodiments, said animal protein population comprises one or more proteins with an amide group. In some embodiments, at least 1% of proteins in said animal protein population comprise an amide group. In some embodiments, at least 2% of proteins in said animal protein population comprise an amide group. In some embodiments, at least 3% of proteins in said animal protein population comprise an amide group. In some embodiments, at least 4% of proteins in said animal protein population comprise an amide group. In some embodiments, at least 5% of proteins in said animal protein population comprise an amide group. In some embodiments, at least 6% of proteins in said animal protein population comprise an amide group. In some embodiments, at least 7% of proteins in said animal protein population comprise an amide group. In some embodiments, at least 8% of proteins in said animal protein population comprise an amide group. In some embodiments, at least 9% of proteins in said animal protein population comprise an amide group. In some embodiments, at least 10% of proteins in said animal protein population comprise an amide group. In some embodiments, at least 11% of proteins in said animal protein population comprise an amide group. In some embodiments, at least 12% of proteins in said animal protein population comprise an amide group. In some embodiments, at least 13% of proteins in said animal protein population comprise an amide group. In some embodiments, at least 14% of proteins in said animal protein population comprise an amide group. In some embodiments, at least 15% of proteins in said animal protein population comprise an amide group. In some embodiments, at least 16% of proteins in said animal protein population comprise an amide group. In some embodiments, at least 17% of proteins in said animal protein population comprise an amide group. In some embodiments, at least 18% of proteins in said animal protein population comprise an amide group. In some embodiments, at least 19% of proteins in said animal protein population comprise an amide group. In some embodiments, at least 20% of proteins in said animal protein population comprise an amide group. In some embodiments, at least 23% of proteins in said animal protein population comprise an amide group. In some embodiments, at least 25% of proteins in said animal protein population comprise an amide group. In some embodiments, at least 27% of proteins in said animal protein population comprise an amide group. In some embodiments, at least 30% of proteins in said animal protein population comprise an amide group. In some embodiments, at least 33% of proteins in said animal protein population comprise an amide group. In some embodiments, at least 35% of proteins in said animal protein population comprise an amide group. In some embodiments, at least 37% of proteins in said animal protein population comprise an amide group. In some embodiments, at least 40% of proteins in said animal protein population comprise an amide group. In some embodiments, at least 42% of proteins in said animal protein population comprise an amide group. In some embodiments, at least 45% of proteins in said animal protein population comprise an amide group. In some embodiments, at least 47% of proteins in said animal protein population comprise an amide group. In some embodiments, at least 50% of proteins in said animal protein population comprise an amide group. In some embodiments, at least 52% of proteins in said animal protein population comprise an amide group. In some embodiments, at least 55% of proteins in said animal protein population comprise an amide group. In some embodiments, at least 57% of proteins in said animal protein population comprise an amide group. In some embodiments, at least 60% of proteins in said animal protein population comprise an amide group. In some embodiments, the amount of the animal protein population that comprises an amide group is about 60% to about 100%. In some embodiments, the amount of the animal protein population that comprises an amide group is about 60% to about 65%, about 60% to about 70%, about 60% to about 75%, about 60% to about 80%, about 60% to about 85%, about 60% to about 90%, about 60% to about 95%, about 60% to about 100%, about 65% to about 70%, about 65% to about 75%, about 65% to about 80%, about 65% to about 85%, about 65% to about 90%, about 65% to about 95%, about 65% to about 100%, about 70% to about 75%, about 70% to about 80%, about 70% to about 85%, about 70% to about 90%, about 70% to about 95%, about 70% to about 100%, about 75% to about 80%, about 75% to about 85%, about 75% to about 90%, about 75% to about 95%, about 75% to about 100%, about 80% to about 85%, about 80% to about 90%, about 80% to about 95%, about 80% to about 100%, about 85% to about 90%, about 85% to about 95%, about 85% to about 100%, about 90% to about 95%, about 90% to about 100%, or about 95% to about 100%. In some embodiments, the amount of the animal protein population that comprises an amide group is about 60%, about 65%, about 70%, about 75%, about 80%, about 85%, about 90%, about 95%, or about 100%. In some embodiments, the amount of the animal protein population that comprises an amide group is at least about 60%, about 65%, about 70%, about 75%, about 80%, about 85%, about 90%, or about 95%. In some embodiments, the amount of the animal protein population that comprises an amide group is at most about 65%, about 70%, about 75%, about 80%, about 85%, about 90%, about 95%, or about 100%.

The Protein Digestibility-Corrected Amino Acid Score (PDCAAS) is a method for evaluation of protein quality that is described in Protein Quality Evaluation, Food and Nutrition Paper 51, Rome, Italy: FAO/WHO, 1991, p 35. To calculate a PDCAAS value, a food is analyzed for proximate and amino acid composition. A protein digestibility value is obtained from a data base or determined by the rat balance method. An amino acid score is determined as follows: Amino Acid Score=MCI of essential amino acid in 1.0 q test protein mg of essential amino acid in 1.0 g reference pattern. The PDCAAS is then calculated by multiplying the lowest amino acid score×true protein digestibility: PDCAAS=Lowest Amino Acid Score×True Digestibility. Scores above 1.00 are considered as 1.00.

In certain embodiments, the protein compositions of the disclosure have PDCAAS score greater than 0, for example greater than 0.5, greater than 0.6, greater than 0.7, greater than 0.8, greater than 0.9, greater than 0.97, or greater than 0.99. In other embodiments of the disclosure, the protein compositions have a PDCAAS score in the range of from about 0.6 to about 1.0. In other embodiments, PDCAAS score is in the range of 0.75 to 1.0. In some embodiments, the PDCAAS score is in the range of from 0.85 to 1.0. In some embodiments, the PDCAAS score is 1 (or greater than one, which is considered as 1).

In various embodiments, the protein used in the protein compositions disclosed herein comprises one or more essential amino acids, i.e. one or more amino acids selected from the group consisting of phenylalanine, valine, threonine, tryptophan, methionine, leucine, isoleucine, lysine, and histidine. In some embodiments, the protein used in the protein compositions disclosed herein comprises all essential amino acids. In some embodiments, the protein used in the protein compositions disclosed herein further comprises one or more non-essential amino acids, for example one or more of cysteine, aspartic acid, serine, glutamic acid, proline, glycine, alanine, tyrosine, and arginine. In some embodiments, the protein used in the protein compositions disclosed herein comprises all of phenylalanine, valine, threonine, tryptophan, methionine, leucine, isoleucine, lysine, histidine, cysteine, aspartic acid, serine, glutamic acid, proline, glycine, alanine, tyrosine, and arginine.

In some embodiments, said animal protein population is water insoluble. In some embodiments, said animal protein population is non-hygroscopic.

In some embodiments, the protein in the protein in the protein compositions described herein has a solubility of less than 50%, less than 40%, less than 30%, less than 20%, less than 15%, less than 10%, less than 7%, less than 5%, less than 3%, less than 2%, less than 1%, or less than 0.5% at a pH range of about 1-8, for example at a pH of 7. In some embodiments, the protein in the protein compositions disclosed herein are non-hygroscopic i.e. they do not adsorb moisture from the surrounding atmosphere.

As previously indicated, in various embodiments, the protein compositions disclosed herein are water insoluble. As such the protein compositions disclosed herein comprise essentially no water. As such, the protein compositions of the disclosure have a long shelf-life. The term shelf-life as used herein means a period of time for which the protein compositions can be stored for, at ambient temperature, while maintaining substantially the same physical and chemical properties, e.g. taste, smell, color, and the like. In some examples, protein compositions disclosed herein have a shelf life of at least about 0.5 years to about 15 years. In some examples, protein compositions disclosed herein have a shelf life of at least about 0.5 years. In some examples, protein compositions disclosed herein have a shelf life of at most about 15 years. In some examples, protein compositions disclosed herein have a shelf life of at least about 0.5 years to about 1 year, about 0.5 years to about 1.5 years, about 0.5 years to about 2 years, about 0.5 years to about 2.5 years, about 0.5 years to about 3 years, about 0.5 years to about 2.5 years, about 0.5 years to about 4 years, about 0.5 years to about 4.5 years, about 0.5 years to about 5 years, about 0.5 years to about 10 years, about 0.5 years to about 15 years, about 1 year to about 1.5 years, about 1 year to about 2 years, about 1 year to about 2.5 years, about 1 year to about 3 years, about 1 year to about 2.5 years, about 1 year to about 4 years, about 1 year to about 4.5 years, about 1 year to about 5 years, about 1 year to about 10 years, about 1 year to about 15 years, about 1.5 years to about 2 years, about 1.5 years to about 2.5 years, about 1.5 years to about 3 years, about 1.5 years to about 2.5 years, about 1.5 years to about 4 years, about 1.5 years to about 4.5 years, about 1.5 years to about 5 years, about 1.5 years to about 10 years, about 1.5 years to about 15 years, about 2 years to about 2.5 years, about 2 years to about 3 years, about 2 years to about 2.5 years, about 2 years to about 4 years, about 2 years to about 4.5 years, about 2 years to about 5 years, about 2 years to about 10 years, about 2 years to about 15 years, about 2.5 years to about 3 years, about 2.5 years to about 2.5 years, about 2.5 years to about 4 years, about 2.5 years to about 4.5 years, about 2.5 years to about 5 years, about 2.5 years to about 10 years, about 2.5 years to about 15 years, about 3 years to about 2.5 years, about 3 years to about 4 years, about 3 years to about 4.5 years, about 3 years to about 5 years, about 3 years to about 10 years, about 3 years to about 15 years, about 2.5 years to about 4 years, about 2.5 years to about 4.5 years, about 2.5 years to about 5 years, about 2.5 years to about 10 years, about 2.5 years to about 15 years, about 4 years to about 4.5 years, about 4 years to about 5 years, about 4 years to about 10 years, about 4 years to about 15 years, about 4.5 years to about 5 years, about 4.5 years to about 10 years, about 4.5 years to about 15 years, about 5 years to about 10 years, about 5 years to about 15 years, or about 10 years to about 15 years. In some examples, protein compositions disclosed herein have a shelf life of at least about 0.5 years, about 1 year, about 1.5 years, about 2 years, about 2.5 years, about 3 years, about 2.5 years, about 4 years, about 4.5 years, about 5 years, about 10 years, or about 15 years. In some examples, protein compositions disclosed herein have a shelf life of at least about 15 years.

In some embodiments, by virtue of their extended shelf-life and water insolubility, the protein compositions of the present disclosure do not require refrigeration or freezing. The protein compositions are thus suitable for shipping and distribution. The protein compositions can be thus also be purchased in larger, more convenient and/or cost-effective quantities, as the quality of the protein compositions will be maintained for long periods of time.

In some embodiments, said animal protein population comprises one or more proteins with an aldehyde group. In some embodiments, at least 1% of proteins in said animal protein population comprise an aldehyde group. In some embodiments, at least 2% of proteins in said animal protein population comprise an aldehyde group. In some embodiments, at least 3% of proteins in said animal protein population comprise an aldehyde group. In some embodiments, at least 4% of proteins in said animal protein population comprise an aldehyde group. In some embodiments, at least 5% of proteins in said animal protein population comprise an aldehyde group. In some embodiments, at least 6% of proteins in said animal protein population comprise an aldehyde group. In some embodiments, at least 7% of proteins in said animal protein population comprise an aldehyde group. In some embodiments, at least 8% of proteins in said animal protein population comprise an aldehyde group. In some embodiments, at least 9% of proteins in said animal protein population comprise an aldehyde group. In some embodiments, at least 10% of proteins in said animal protein population comprise an aldehyde group. In some embodiments, at least 11% of proteins in said animal protein population comprise an aldehyde group. In some embodiments, at least 12% of proteins in said animal protein population comprise an aldehyde group. In some embodiments, at least 13% of proteins in said animal protein population comprise an aldehyde group. In some embodiments, at least 14% of proteins in said animal protein population comprise an aldehyde group. In some embodiments, at least 15% of proteins in said animal protein population comprise an aldehyde group. In some embodiments, at least 16% of proteins in said animal protein population comprise an aldehyde group. In some embodiments, at least 17% of proteins in said animal protein population comprise an aldehyde group. In some embodiments, at least 18% of proteins in said animal protein population comprise an aldehyde group. In some embodiments, at least 19% of proteins in said animal protein population comprise an aldehyde group. In some embodiments, at least 20% of proteins in said animal protein population comprise an aldehyde group. In some embodiments, at least 23% of proteins in said animal protein population comprise an aldehyde group. In some embodiments, at least 25% of proteins in said animal protein population comprise an aldehyde group. In some embodiments, at least 27% of proteins in said animal protein population comprise an aldehyde group. In some embodiments, at least 30% of proteins in said animal protein population comprise an aldehyde group. In some embodiments, at least 33% of proteins in said animal protein population comprise an aldehyde group. In some embodiments, at least 35% of proteins in said animal protein population comprise an aldehyde group. In some embodiments, at least 37% of proteins in said animal protein population comprise an aldehyde group. In some embodiments, at least 40% of proteins in said animal protein population comprise an aldehyde group. In some embodiments, at least 42% of proteins in said animal protein population comprise an aldehyde group. In some embodiments, at least 45% of proteins in said animal protein population comprise an aldehyde group. In some embodiments, at least 47% of proteins in said animal protein population comprise an aldehyde group. In some embodiments, at least 50% of proteins in said animal protein population comprise an aldehyde group. In some embodiments, at least 52% of proteins in said animal protein population comprise an aldehyde group. In some embodiments, at least 55% of proteins in said animal protein population comprise an aldehyde group. In some embodiments, at least 57% of proteins in said animal protein population comprise an aldehyde group. In some embodiments, at least 60% of proteins in said animal protein population comprise an aldehyde group. In some embodiments, the amount of the animal protein population that comprises an aldehyde group is about 60% to about 100%. In some embodiments, the amount of the animal protein population that comprises an aldehyde group is about 60% to about 65%, about 60% to about 70%, about 60% to about 75%, about 60% to about 80%, about 60% to about 85%, about 60% to about 90%, about 60% to about 95%, about 60% to about 100%, about 65% to about 70%, about 65% to about 75%, about 65% to about 80%, about 65% to about 85%, about 65% to about 90%, about 65% to about 95%, about 65% to about 100%, about 70% to about 75%, about 70% to about 80%, about 70% to about 85%, about 70% to about 90%, about 70% to about 95%, about 70% to about 100%, about 75% to about 80%, about 75% to about 85%, about 75% to about 90%, about 75% to about 95%, about 75% to about 100%, about 80% to about 85%, about 80% to about 90%, about 80% to about 95%, about 80% to about 100%, about 85% to about 90%, about 85% to about 95%, about 85% to about 100%, about 90% to about 95%, about 90% to about 100%, or about 95% to about 100%. In some embodiments, the amount of the animal protein population that comprises an aldehyde group is about 60%, about 65%, about 70%, about 75%, about 80%, about 85%, about 90%, about 95%, or about 100%. In some embodiments, the amount of the animal protein population that comprises an aldehyde group is at least about 60%, about 65%, about 70%, about 75%, about 80%, about 85%, about 90%, or about 95%. In some embodiments, the amount of the animal protein population that comprises an aldehyde group is at most about 65%, about 70%, about 75%, about 80%, about 85%, about 90%, about 95%, or about 100%.

In some embodiments, said animal protein population comprises one or more proteins with a triazene group. In some embodiments, at least 1% of proteins in said animal protein population comprise a triazene group. In some embodiments, at least 2% of proteins in said animal protein population comprise a triazene group. In some embodiments, at least 3% of proteins in said animal protein population comprise a triazene group. In some embodiments, at least 4% of proteins in said animal protein population comprise a triazene group. In some embodiments, at least 5% of proteins in said animal protein population comprise a triazene group. In some embodiments, at least 6% of proteins in said animal protein population comprise a triazene group. In some embodiments, at least 7% of proteins in said animal protein population comprise a triazene group. In some embodiments, at least 8% of proteins in said animal protein population comprise a triazene group. In some embodiments, at least 9% of proteins in said animal protein population comprise a triazene group. In some embodiments, at least 10% of proteins in said animal protein population comprise a triazene group. In some embodiments, at least 11% of proteins in said animal protein population comprise a triazene group. In some embodiments, at least 12% of proteins in said animal protein population comprise a triazene group. In some embodiments, at least 13% of proteins in said animal protein population comprise a triazene group. In some embodiments, at least 14% of proteins in said animal protein population comprise a triazene group. In some embodiments, at least 15% of proteins in said animal protein population comprise a triazene group. In some embodiments, at least 16% of proteins in said animal protein population comprise a triazene group. In some embodiments, at least 17% of proteins in said animal protein population comprise a triazene group. In some embodiments, at least 18% of proteins in said animal protein population comprise a triazene group. In some embodiments, at least 19% of proteins in said animal protein population comprise a triazene group. In some embodiments, at least 20% of proteins in said animal protein population comprise a triazene group. In some embodiments, at least 23% of proteins in said animal protein population comprise a triazene group. In some embodiments, at least 25% of proteins in said animal protein population comprise a triazene group. In some embodiments, at least 27% of proteins in said animal protein population comprise a triazene group. In some embodiments, at least 30% of proteins in said animal protein population comprise a triazene group. In some embodiments, at least 33% of proteins in said animal protein population comprise a triazene group. In some embodiments, at least 35% of proteins in said animal protein population comprise a triazene group. In some embodiments, at least 37% of proteins in said animal protein population comprise a triazene group. In some embodiments, at least 40% of proteins in said animal protein population comprise a triazene group. In some embodiments, at least 42% of proteins in said animal protein population comprise a triazene group. In some embodiments, at least 45% of proteins in said animal protein population comprise a triazene group. In some embodiments, at least 47% of proteins in said animal protein population comprise a triazene group. In some embodiments, at least 50% of proteins in said animal protein population comprise a triazene group. In some embodiments, at least 52% of proteins in said animal protein population comprise a triazene group. In some embodiments, at least 55% of proteins in said animal protein population comprise a triazene group. In some embodiments, at least 57% of proteins in said animal protein population comprise a triazene group. In some embodiments, at least 60% of proteins in said animal protein population comprise a triazene group. In some embodiments, the amount of the animal protein population that comprises a triazene group is about 60% to about 100%. In some embodiments, the amount of the animal protein population that comprises a triazene group is about 60% to about 65%, about 60% to about 70%, about 60% to about 75%, about 60% to about 80%, about 60% to about 85%, about 60% to about 90%, about 60% to about 95%, about 60% to about 100%, about 65% to about 70%, about 65% to about 75%, about 65% to about 80%, about 65% to about 85%, about 65% to about 90%, about 65% to about 95%, about 65% to about 100%, about 70% to about 75%, about 70% to about 80%, about 70% to about 85%, about 70% to about 90%, about 70% to about 95%, about 70% to about 100%, about 75% to about 80%, about 75% to about 85%, about 75% to about 90%, about 75% to about 95%, about 75% to about 100%, about 80% to about 85%, about 80% to about 90%, about 80% to about 95%, about 80% to about 100%, about 85% to about 90%, about 85% to about 95%, about 85% to about 100%, about 90% to about 95%, about 90% to about 100%, or about 95% to about 100%. In some embodiments, the amount of the animal protein population that comprises a triazene group is about 60%, about 65%, about 70%, about 75%, about 80%, about 85%, about 90%, about 95%, or about 100%. In some embodiments, the amount of the animal protein population that comprises a triazene group is at least about 60%, about 65%, about 70%, about 75%, about 80%, about 85%, about 90%, or about 95%. In some embodiments, the amount of the animal protein population that comprises a triazene group is at most about 65%, about 70%, about 75%, about 80%, about 85%, about 90%, about 95%, or about 100%.

In some embodiments, at least 1% of proteins in said animal protein population comprise a carboxylic acid group. In some embodiments, at least 2% of proteins in said animal protein population comprise a carboxylic acid group. In some embodiments, at least 3% of proteins in said animal protein population comprise a carboxylic acid group. In some embodiments, at least 4% of proteins in said animal protein population comprise a carboxylic acid group. In some embodiments, at least 5% of proteins in said animal protein population comprise a carboxylic acid group. In some embodiments, at least 6% of proteins in said animal protein population comprise a carboxylic acid group. In some embodiments, at least 7% of proteins in said animal protein population comprise a carboxylic acid group. In some embodiments, at least 8% of proteins in said animal protein population comprise a carboxylic acid group. In some embodiments, at least 9% of proteins in said animal protein population comprise a carboxylic acid group. In some embodiments, at least 10% of proteins in said animal protein population comprise a carboxylic acid group. In some embodiments, at least 11% of proteins in said animal protein population comprise a carboxylic acid group. In some embodiments, at least 12% of proteins in said animal protein population comprise a carboxylic acid group. In some embodiments, at least 13% of proteins in said animal protein population comprise a carboxylic acid group. In some embodiments, at least 14% of proteins in said animal protein population comprise a carboxylic acid group. In some embodiments, at least 15% of proteins in said animal protein population comprise a carboxylic acid group. In some embodiments, at least 16% of proteins in said animal protein population comprise a carboxylic acid group. In some embodiments, at least 17% of proteins in said animal protein population comprise a carboxylic acid group. In some embodiments, at least 18% of proteins in said animal protein population comprise a carboxylic acid group. In some embodiments, at least 19% of proteins in said animal protein population comprise a carboxylic acid group. In some embodiments, at least 20% of proteins in said animal protein population comprise a carboxylic acid group. In some embodiments, at least 23% of proteins in said animal protein population comprise a carboxylic acid group. In some embodiments, at least 25% of proteins in said animal protein population comprise a carboxylic acid group. In some embodiments, at least 27% of proteins in said animal protein population comprise a carboxylic acid group. In some embodiments, at least 30% of proteins in said animal protein population comprise a carboxylic acid group. In some embodiments, at least 33% of proteins in said animal protein population comprise a carboxylic acid group. In some embodiments, at least 35% of proteins in said animal protein population comprise a carboxylic acid group. In some embodiments, at least 37% of proteins in said animal protein population comprise a carboxylic acid group. In some embodiments, at least 40% of proteins in said animal protein population comprise a carboxylic acid group. In some embodiments, at least 42% of proteins in said animal protein population comprise a carboxylic acid group. In some embodiments, at least 45% of proteins in said animal protein population comprise a carboxylic acid group. In some embodiments, at least 47% of proteins in said animal protein population comprise a carboxylic acid group. In some embodiments, at least 50% of proteins in said animal protein population comprise a carboxylic acid group. In some embodiments, at least 52% of proteins in said animal protein population comprise a carboxylic acid group. In some embodiments, at least 55% of proteins in said animal protein population comprise a carboxylic acid group. In some embodiments, at least 57% of proteins in said animal protein population comprise a carboxylic acid group. In some embodiments, at least 60% of proteins in said animal protein population comprise a carboxylic acid group. In some embodiments, the amount of the animal protein population that comprises a carboxylic acid group is about 60% to about 100%. In some embodiments, the amount of the animal protein population that comprises a carboxylic acid group is about 60% to about 65%, about 60% to about 70%, about 60% to about 75%, about 60% to about 80%, about 60% to about 85%, about 60% to about 90%, about 60% to about 95%, about 60% to about 100%, about 65% to about 70%, about 65% to about 75%, about 65% to about 80%, about 65% to about 85%, about 65% to about 90%, about 65% to about 95%, about 65% to about 100%, about 70% to about 75%, about 70% to about 80%, about 70% to about 85%, about 70% to about 90%, about 70% to about 95%, about 70% to about 100%, about 75% to about 80%, about 75% to about 85%, about 75% to about 90%, about 75% to about 95%, about 75% to about 100%, about 80% to about 85%, about 80% to about 90%, about 80% to about 95%, about 80% to about 100%, about 85% to about 90%, about 85% to about 95%, about 85% to about 100%, about 90% to about 95%, about 90% to about 100%, or about 95% to about 100%. In some embodiments, the amount of the animal protein population that comprises a carboxylic acid group is about 60%, about 65%, about 70%, about 75%, about 80%, about 85%, about 90%, about 95%, or about 100%. In some embodiments, the amount of the animal protein population that comprises a carboxylic acid group is at least about 60%, about 65%, about 70%, about 75%, about 80%, about 85%, about 90%, or about 95%. In some embodiments, the amount of the animal protein population that comprises a carboxylic acid group is at most about 65%, about 70%, about 75%, about 80%, about 85%, about 90%, about 95%, or about 100%.

In some embodiments, said animal protein population comprises one or more proteins with an alkene group. In some embodiments, said alkene group is a trans-alkene group. In some embodiments, at least 1% of proteins in said animal protein population comprise an alkene group. In some embodiments, at least 2% of proteins in said animal protein population comprise an alkene group. In some embodiments, at least 3% of proteins in said animal protein population comprise an alkene group. In some embodiments, at least 4% of proteins in said animal protein population comprise an alkene group. In some embodiments, at least 5% of proteins in said animal protein population comprise an alkene group. In some embodiments, at least 6% of proteins in said animal protein population comprise an alkene group. In some embodiments, at least 7% of proteins in said animal protein population comprise an alkene group. In some embodiments, at least 8% of proteins in said animal protein population comprise an alkene group. In some embodiments, at least 9% of proteins in said animal protein population comprise an alkene group. In some embodiments, at least 10% of proteins in said animal protein population comprise an alkene group. In some embodiments, at least 11% of proteins in said animal protein population comprise an alkene group. In some embodiments, at least 12% of proteins in said animal protein population comprise an alkene group. In some embodiments, at least 13% of proteins in said animal protein population comprise an alkene group. In some embodiments, at least 14% of proteins in said animal protein population comprise an alkene group. In some embodiments, at least 15% of proteins in said animal protein population comprise an alkene group. In some embodiments, at least 16% of proteins in said animal protein population comprise an alkene group. In some embodiments, at least 17% of proteins in said animal protein population comprise an alkene group. In some embodiments, at least 18% of proteins in said animal protein population comprise an alkene group. In some embodiments, at least 19% of proteins in said animal protein population comprise an alkene group. In some embodiments, at least 20% of proteins in said animal protein population comprise an alkene group. In some embodiments, at least 23% of proteins in said animal protein population comprise an alkene group. In some embodiments, at least 25% of proteins in said animal protein population comprise an alkene group. In some embodiments, at least 27% of proteins in said animal protein population comprise an alkene group. In some embodiments, at least 30% of proteins in said animal protein population comprise an alkene group. In some embodiments, at least 33% of proteins in said animal protein population comprise an alkene group. In some embodiments, at least 35% of proteins in said animal protein population comprise an alkene group. In some embodiments, at least 37% of proteins in said animal protein population comprise an alkene group. In some embodiments, at least 40% of proteins in said animal protein population comprise an alkene group. In some embodiments, at least 42% of proteins in said animal protein population comprise an alkene group. In some embodiments, at least 45% of proteins in said animal protein population comprise an alkene group. In some embodiments, at least 47% of proteins in said animal protein population comprise an alkene group. In some embodiments, at least 50% of proteins in said animal protein population comprise an alkene group. In some embodiments, at least 52% of proteins in said animal protein population comprise an alkene group. In some embodiments, at least 55% of proteins in said animal protein population comprise an alkene group. In some embodiments, at least 57% of proteins in said animal protein population comprise an alkene group. In some embodiments, at least 60% of proteins in said animal protein population comprise an alkene group. In some embodiments, the amount of the animal protein population that comprises an alkene group is about 60% to about 100%. In some embodiments, the amount of the animal protein population that comprises an alkene group is about 60% to about 65%, about 60% to about 70%, about 60% to about 75%, about 60% to about 80%, about 60% to about 85%, about 60% to about 90%, about 60% to about 95%, about 60% to about 100%, about 65% to about 70%, about 65% to about 75%, about 65% to about 80%, about 65% to about 85%, about 65% to about 90%, about 65% to about 95%, about 65% to about 100%, about 70% to about 75%, about 70% to about 80%, about 70% to about 85%, about 70% to about 90%, about 70% to about 95%, about 70% to about 100%, about 75% to about 80%, about 75% to about 85%, about 75% to about 90%, about 75% to about 95%, about 75% to about 100%, about 80% to about 85%, about 80% to about 90%, about 80% to about 95%, about 80% to about 100%, about 85% to about 90%, about 85% to about 95%, about 85% to about 100%, about 90% to about 95%, about 90% to about 100%, or about 95% to about 100%. In some embodiments, the amount of the animal protein population that comprises an alkene group is about 60%, about 65%, about 70%, about 75%, about 80%, about 85%, about 90%, about 95%, or about 100%. In some embodiments, the amount of the animal protein population that comprises an alkene group is at least about 60%, about 65%, about 70%, about 75%, about 80%, about 85%, about 90%, or about 95%. In some embodiments, the amount of the animal protein population that comprises an alkene group is at most about 65%, about 70%, about 75%, about 80%, about 85%, about 90%, about 95%, or about 100%.

In some embodiments, at least 1% of proteins in said animal protein population comprise an alkane group. In some embodiments, at least 2% of proteins in said animal protein population comprise an alkane group. In some embodiments, at least 3% of proteins in said animal protein population comprise an alkane group. In some embodiments, at least 4% of proteins in said animal protein population comprise an alkane group. In some embodiments, at least 5% of proteins in said animal protein population comprise an alkane group. In some embodiments, at least 6% of proteins in said animal protein population comprise an alkane group. In some embodiments, at least 7% of proteins in said animal protein population comprise an alkane group. In some embodiments, at least 8% of proteins in said animal protein population comprise an alkane group. In some embodiments, at least 9% of proteins in said animal protein population comprise an alkane group. In some embodiments, at least 10% of proteins in said animal protein population comprise an alkane group. In some embodiments, at least 11% of proteins in said animal protein population comprise an alkane group. In some embodiments, at least 12% of proteins in said animal protein population comprise an alkane group. In some embodiments, at least 13% of proteins in said animal protein population comprise an alkane group. In some embodiments, at least 14% of proteins in said animal protein population comprise an alkane group. In some embodiments, at least 15% of proteins in said animal protein population comprise an alkane group. In some embodiments, at least 16% of proteins in said animal protein population comprise an alkane group. In some embodiments, at least 17% of proteins in said animal protein population comprise an alkane group. In some embodiments, at least 18% of proteins in said animal protein population comprise an alkane group. In some embodiments, at least 19% of proteins in said animal protein population comprise an alkane group. In some embodiments, at least 20% of proteins in said animal protein population comprise an alkane group. In some embodiments, at least 23% of proteins in said animal protein population comprise an alkane group. In some embodiments, at least 25% of proteins in said animal protein population comprise an alkane group. In some embodiments, at least 27% of proteins in said animal protein population comprise an alkane group. In some embodiments, at least 30% of proteins in said animal protein population comprise an alkane group. In some embodiments, at least 33% of proteins in said animal protein population comprise an alkane group. In some embodiments, at least 35% of proteins in said animal protein population comprise an alkane group. In some embodiments, at least 37% of proteins in said animal protein population comprise an alkane group. In some embodiments, at least 40% of proteins in said animal protein population comprise an alkane group. In some embodiments, at least 42% of proteins in said animal protein population comprise an alkane group. In some embodiments, at least 45% of proteins in said animal protein population comprise an alkane group. In some embodiments, at least 47% of proteins in said animal protein population comprise an alkane group. In some embodiments, at least 50% of proteins in said animal protein population comprise an alkane group. In some embodiments, at least 52% of proteins in said animal protein population comprise an alkane group. In some embodiments, at least 55% of proteins in said animal protein population comprise an alkane group. In some embodiments, at least 57% of proteins in said animal protein population comprise an alkane group. In some embodiments, at least 60% of proteins in said animal protein population comprise an alkane group. In some embodiments, the amount of the animal protein population that comprises an alkane group is about 60% to about 100%. In some embodiments, the amount of the animal protein population that comprises an alkane group is about 60% to about 65%, about 60% to about 70%, about 60% to about 75%, about 60% to about 80%, about 60% to about 85%, about 60% to about 90%, about 60% to about 95%, about 60% to about 100%, about 65% to about 70%, about 65% to about 75%, about 65% to about 80%, about 65% to about 85%, about 65% to about 90%, about 65% to about 95%, about 65% to about 100%, about 70% to about 75%, about 70% to about 80%, about 70% to about 85%, about 70% to about 90%, about 70% to about 95%, about 70% to about 100%, about 75% to about 80%, about 75% to about 85%, about 75% to about 90%, about 75% to about 95%, about 75% to about 100%, about 80% to about 85%, about 80% to about 90%, about 80% to about 95%, about 80% to about 100%, about 85% to about 90%, about 85% to about 95%, about 85% to about 100%, about 90% to about 95%, about 90% to about 100%, or about 95% to about 100%. In some embodiments, the amount of the animal protein population that comprises an alkane group is about 60%, about 65%, about 70%, about 75%, about 80%, about 85%, about 90%, about 95%, or about 100%. In some embodiments, the amount of the animal protein population that comprises an alkane group is at least about 60%, about 65%, about 70%, about 75%, about 80%, about 85%, about 90%, or about 95%. In some embodiments, the amount of the animal protein population that comprises an alkane group is at most about 65%, about 70%, about 75%, about 80%, about 85%, about 90%, about 95%, or about 100%.

In some embodiments, at least 5% of proteins in said animal protein population are bioactive proteins. In some embodiments, at least 10% of proteins in said animal protein population are bioactive proteins. In some embodiments, at least 15% of proteins in said animal protein population are bioactive proteins. In some embodiments, at least 20% of proteins in said animal protein population are bioactive proteins. In some embodiments, at least 25% of proteins in said animal protein population are bioactive proteins. In some embodiments, at least 30% of proteins in said animal protein population are bioactive proteins. In some embodiments, at least 35% of proteins in said animal protein population are bioactive proteins. In some embodiments, at least 40% of proteins in said animal protein population are bioactive proteins. In some embodiments, at least 45% of proteins in said animal protein population are bioactive proteins. In some embodiments, at least 50% of proteins in said animal protein population are bioactive proteins. In some embodiments, at least 55% of proteins in said animal protein population are bioactive proteins. In some embodiments, at least 60% of proteins in said animal protein population are bioactive proteins. In some embodiments, at least 65% of proteins in said animal protein population are bioactive proteins. In some embodiments, at least 70% of proteins in said animal protein population are bioactive proteins. In some embodiments, at least 75% of proteins in said animal protein population are bioactive proteins. In some embodiments, at least 80% of proteins in said animal protein population are bioactive proteins. In some embodiments, at least 85% of proteins in said animal protein population are bioactive proteins. In some embodiments, at least 88% of proteins in said animal protein population are bioactive proteins. In some embodiments, at least 90% of proteins in said animal protein population are bioactive proteins. In some embodiments, at least 91% of proteins in said animal protein population are bioactive proteins. In some embodiments, at least 92% of proteins in said animal protein population are bioactive proteins. In some embodiments, at least 93% of proteins in said animal protein population are bioactive proteins. In some embodiments, at least 94% of proteins in said animal protein population are bioactive proteins. In some embodiments, at least 95% of proteins in said animal protein population are bioactive proteins. In some embodiments, at least 96% of proteins in said animal protein population are bioactive proteins. In some embodiments, at least 97% of proteins in said animal protein population are bioactive proteins. In some embodiments, at least 98% of proteins in said animal protein population are bioactive proteins. In some embodiments, at least 99% of proteins in said animal protein population are bioactive proteins.

In some embodiments, said animal protein population is suitable for human consumption.

In some embodiments, said animal protein population has antihypersensitivity, antithrombotic, antioxidant, antimicrobial, antiproliferative, or anticoagulative properties. In some embodiments, said animal protein population has antihypersensitivity properties. In some embodiments, said animal protein population has antithrombotic properties. In some embodiments, said animal protein population has antioxidant properties. In some embodiments, said animal protein population has antimicrobial properties. In some embodiments, said animal protein population has antiproliferative properties. In some embodiments, said animal protein population has anticoagulative properties.

In some embodiments, said animal protein population is an angiotensin-converting-enzyme inhibitor.

In another aspect, described herein is a composition comprising a protein population, wherein greater than 30% of proteins in said animal protein population having from 50 to 60 amino acids. In some embodiments, greater than 35% of proteins in said animal protein population have from 50 to 60 amino acids. In some embodiments, greater than 40% of proteins in said animal protein population have from 50 to 60 amino acids. In some embodiments, greater than 45% of proteins in said animal protein population have from 50 to 60 amino acids. In some embodiments, greater than 50% of proteins in said animal protein population have from 50 to 60 amino acids. In some embodiments, greater than 55% of proteins in said animal protein population have from 50 to 60 amino acids. In some embodiments, greater than 60% of proteins in said animal protein population have from 50 to 60 amino acids. In some embodiments, greater than 65% of proteins in said animal protein population have from 50 to 60 amino acids. In some embodiments, greater than 70% of proteins in said animal protein population have from 50 to 60 amino acids. In some embodiments, greater than 75% of proteins in said animal protein population have from 50 to 60 amino acids. In some embodiments, greater than 80% of proteins in said animal protein population have from 50 to 60 amino acids. In some embodiments, greater than 85% of proteins in said animal protein population have from 50 to 60 amino acids. In some embodiments, greater than 90% of proteins in said animal protein population have from 50 to 60 amino acids. In some embodiments, greater than 91% of proteins in said animal protein population have from 50 to 60 amino acids. In some embodiments, greater than 92% of proteins in said animal protein population have from 50 to 60 amino acids. In some embodiments, greater than 93% of proteins in said animal protein population have from 50 to 60 amino acids. In some embodiments, greater than 94% of proteins in said animal protein population have from 50 to 60 amino acids. In some embodiments, greater than 95% of proteins in said animal protein population have from 50 to 60 amino acids. In some embodiments, greater than 96% of proteins in said animal protein population have from 50 to 60 amino acids. In some embodiments, greater than 97% of proteins in said animal protein population have from 50 to 60 amino acids. In some embodiments, greater than 98% of proteins in said animal protein population have from 50 to 60 amino acids. In some embodiments, greater than 99% of proteins in said animal protein population have from 50 to 60 amino acids.

In some embodiments, greater than 35% of proteins in said animal protein population have from 40 to 60 amino acids. In some embodiments, greater than 40% of proteins in said animal protein population have from 40 to 60 amino acids. In some embodiments, greater than 45% of proteins in said animal protein population have from 40 to 60 amino acids. In some embodiments, greater than 50% of proteins in said animal protein population have from 40 to 60 amino acids. In some embodiments, greater than 55% of proteins in said animal protein population have from 40 to 60 amino acids. In some embodiments, greater than 60% of proteins in said animal protein population have from 40 to 60 amino acids. In some embodiments, greater than 65% of proteins in said animal protein population have from 40 to 60 amino acids. In some embodiments, greater than 70% of proteins in said animal protein population have from 40 to 60 amino acids. In some embodiments, greater than 75% of proteins in said animal protein population have from 40 to 60 amino acids. In some embodiments, greater than 80% of proteins in said animal protein population have from 40 to 60 amino acids. In some embodiments, greater than 85% of proteins in said animal protein population have from 40 to 60 amino acids. In some embodiments, greater than 90% of proteins in said animal protein population have from 40 to 60 amino acids. In some embodiments, greater than 91% of proteins in said animal protein population have from 40 to 60 amino acids. In some embodiments, greater than 92% of proteins in said animal protein population have from 40 to 60 amino acids. In some embodiments, greater than 93% of proteins in said animal protein population have from 40 to 60 amino acids. In some embodiments, greater than 94% of proteins in said animal protein population have from 40 to 60 amino acids. In some embodiments, greater than 95% of proteins in said animal protein population have from 40 to 60 amino acids. In some embodiments, greater than 96% of proteins in said animal protein population have from 40 to 60 amino acids. In some embodiments, greater than 97% of proteins in said animal protein population have from 40 to 60 amino acids. In some embodiments, greater than 98% of proteins in said animal protein population have from 40 to 60 amino acids. In some embodiments, greater than 99% of proteins in said animal protein population have from 40 to 60 amino acids.

In some embodiments, the protein compositions comprising a protein population described herein comprise a protein powder. In some embodiments, the protein compositions comprising a protein population described herein comprise greater than 20 wt % of a protein powder, for example greater than 30%, greater than 40%, greater than 50%, or greater than 60% protein powder. The protein powder used in the protein compositions of the disclosure may comprise a plant protein, an animal protein, or a mixture thereof. In some examples, the protein powder used in the protein compositions disclosed herein comprises animal protein. In various embodiments the protein powder used in the protein compositions of the disclosure is water insoluble. In some embodiments, the protein powder used in formulations of the disclosure is non-hygroscopic. In some embodiments, the protein powder used in the protein compositions of the disclosure is water insoluble and non-hygroscopic. Additionally, in some examples, the protein powder used in the protein compositions disclosed herein has a complete amino acid profile, comprising all essential amino acids. Furthermore, the protein powder used in the protein composition of the disclosure has a high digestibility.

The protein compositions of the disclosure also comprises fat. In some examples, the fat in the protein compositions disclosed herein comprises omega-3 rich fat, for example omega-3 rich animal oil.

The protein compositions described herein also comprise carbohydrates. In some cases, said carbohydrates are complex carbohydrates. In some examples, the complex carbohydrates additionally comprise fiber.

In some embodiments, about 50 g of the protein composition disclosed herein comprises about 200 to 350 calories, for example about 250 to 350 calories or about 300 to 350 calories per serving.

In various embodiments, the protein compositions of the disclosure have low osmolality. In some embodiments, the protein compositions described herein have an osmolality between about 100 mOsm/kg and 1000 mOsm/kg, for example between 200 mOsm/kg and 1000 mOsm/kg, between 300 mOsm/kg and 1000 mOsm/kg, between 400 mOsm/kg and 1000 mOsm/kg, between 500 mOsm/kg and 1000 mOsm/kg, between 600 mOsm/kg and 1000 mOsm/kg, between 700 mOsm/kg and 1000 mOsm/kg, between 800 mOsm/kg and 1000 mOsm/kg, or between 900 mOsm/kg and 1000 mOsm/kg. In some embodiments, the osmolality of the protein compositions described herein is less than about 300 mOsm/kg, less than about 275 mOsm/kg, less than about 250 mOsm/kg, less than about 240 mOsm/kg, less than about 230 mOsm/kg, less than about 220 mOsm/kg, less than about 210 mOsm/kg, or less than about 200 mOsm/kg.

The protein compositions of the invention will have water activities that are sufficiently low to prevent the growth of most pathogenic and spoilage bacteria. The protein compositions of the disclosure have water activities that are less than or equal to about 0.90. In some embodiments, the protein compositions of the disclosure will a water activity less than about 0.85. In some embodiments the protein compositions will have a water activity not more than about 0.80, not more than about 0.75, or not more than about 0.70.

The Protein Digestibility-Corrected Amino Acid Score (PDCAAS) is a method for evaluation of protein quality that is described in Protein Quality Evaluation, Food and Nutrition Paper 51, Rome, Italy: FAO/WHO, 1991, p 35. To calculate a PDCAAS value, a food is analyzed for proximate and amino acid composition. A protein digestibility value is obtained from a data base or determined by the rat balance method. An amino acid score is determined as follows: Amino Acid Score=MCI of essential amino acid in 1.0 q test protein mg of essential amino acid in 1.0 g reference pattern

The PDCAAS is then calculated by multiplying the lowest amino acid score×true protein digestibility: PDCAAS=Lowest Amino Acid Score×True Digestibility

Scores above 1.00 are considered as 1.00.

In certain embodiments, the protein compositions of the disclosure have PDCAAS score greater than 0, for example greater than 0.5, greater than 0.6, greater than 0.7, greater than 0.8, greater than 0.9, greater than 0.97, or greater than 0.99. In other embodiments of the disclosure, the protein compositions have a PDCAAS score in the range of from about 0.6 to about 1.0. In other embodiments, PDCAAS score is in the range of 0.75 to 1.0. In some embodiments, the PDCAAS score is in the range of from 0.85 to 1.0. In some embodiments, the PDCAAS score is 1 (or greater than one, which is considered as 1).

As previously indicated, in various embodiments, the protein compositions disclosed herein are prepared without addition of water. As such the protein compositions disclosed herein comprise essentially no water. As such, the protein compositions of the disclosure have a long shelf-life. The term shelf-life as used herein means a period of time for which the protein composition can be stored for, at ambient temperature, while maintaining substantially the same physical and chemical properties, e.g. taste, smell, color, and the like. In some examples, protein compositions disclosed herein have a shelf life of at least about 0.5 years to about 15 years. In some examples, protein compositions disclosed herein have a shelf life of at least about 0.5 years. In some examples, protein compositions disclosed herein have a shelf life of at most about 15 years. In some examples, protein compositions disclosed herein have a shelf life of at least about 0.5 years to about 1 year, about 0.5 years to about 1.5 years, about 0.5 years to about 2 years, about 0.5 years to about 2.5 years, about 0.5 years to about 3 years, about 0.5 years to about 2.5 years, about 0.5 years to about 4 years, about 0.5 years to about 4.5 years, about 0.5 years to about 5 years, about 0.5 years to about 10 years, about 0.5 years to about 15 years, about 1 year to about 1.5 years, about 1 year to about 2 years, about 1 year to about 2.5 years, about 1 year to about 3 years, about 1 year to about 2.5 years, about 1 year to about 4 years, about 1 year to about 4.5 years, about 1 year to about 5 years, about 1 year to about 10 years, about 1 year to about 15 years, about 1.5 years to about 2 years, about 1.5 years to about 2.5 years, about 1.5 years to about 3 years, about 1.5 years to about 2.5 years, about 1.5 years to about 4 years, about 1.5 years to about 4.5 years, about 1.5 years to about 5 years, about 1.5 years to about 10 years, about 1.5 years to about 15 years, about 2 years to about 2.5 years, about 2 years to about 3 years, about 2 years to about 2.5 years, about 2 years to about 4 years, about 2 years to about 4.5 years, about 2 years to about 5 years, about 2 years to about 10 years, about 2 years to about 15 years, about 2.5 years to about 3 years, about 2.5 years to about 2.5 years, about 2.5 years to about 4 years, about 2.5 years to about 4.5 years, about 2.5 years to about 5 years, about 2.5 years to about 10 years, about 2.5 years to about 15 years, about 3 years to about 2.5 years, about 3 years to about 4 years, about 3 years to about 4.5 years, about 3 years to about 5 years, about 3 years to about 10 years, about 3 years to about 15 years, about 2.5 years to about 4 years, about 2.5 years to about 4.5 years, about 2.5 years to about 5 years, about 2.5 years to about 10 years, about 2.5 years to about 15 years, about 4 years to about 4.5 years, about 4 years to about 5 years, about 4 years to about 10 years, about 4 years to about 15 years, about 4.5 years to about 5 years, about 4.5 years to about 10 years, about 4.5 years to about 15 years, about 5 years to about 10 years, about 5 years to about 15 years, or about 10 years to about 15 years. In some examples, protein compositions disclosed herein have a shelf life of at least about 0.5 years, about 1 year, about 1.5 years, about 2 years, about 2.5 years, about 3 years, about 2.5 years, about 4 years, about 4.5 years, about 5 years, about 10 years, or about 15 years. In some examples, protein compositions disclosed herein have a shelf life of at least about 15 years.

In some embodiments, by virtue of their extended shelf-life of the protein compositions of the present disclosure do not require refrigeration or freezing. The protein compositions are thus suitable for shipping and distribution. The protein compositions can be thus also be purchased in larger, more convenient and/or cost-effective quantities, as the quality of the protein compositions will be maintained for long periods of time.

In some embodiments, the animal protein population comprises proteins from bonito, salmon, sardine, tuna, Alaskan pollack, yellowfin sole, shark, mackerel, cichlid animal, yellowstripe trevally, sardinelle, or hoki. In some embodiments, said animal protein population comprises proteins from bonito. In some embodiments, said animal protein population comprises proteins from salmon. In some embodiments, said animal protein population comprises proteins from sardine. In some embodiments, said animal protein population comprises proteins from tuna. In some embodiments, said animal protein population comprises proteins from Alaskan pollack. In some embodiments, said animal protein population comprises proteins from yellowfin sole. In some embodiments, said animal protein population comprises proteins from shark. In some embodiments, said animal protein population comprises proteins from mackerel. In some embodiments, said animal protein population comprises proteins from cichlid animal. In some embodiments, said animal protein population comprises proteins from yellowstripe trevally. In some embodiments, said animal protein population comprises proteins from sardinelle. In some embodiments, said animal protein population comprises proteins from hoki.

Natural protein derived from animal may contain pollutants and/or allergens present in the water where the animal that the protein was derived from resided. In some cases, animal naturally comprise compounds that are allergens to humans. Parvalbumin protein is a common animal allergen. An aspect of the present disclosure proteins a protein composition comprising protein derived from animal that comprises reduced levels of pollutants and/or allergens commonly found in natural protein derived from animal.

In some embodiments, the protein used in the protein compositions disclosed herein comprises less than 1 ppm of heavy metals, for example, less than 0.9 ppm, less than 0.8 ppm, less than 0.7 ppm, less than 0.6 ppm, less than 0.5 ppm, less than 0.4 ppm, less than 0.3 ppm, less than 0.2 ppm, less than 0.1 ppm or less than 0.05 ppm of heavy metals.

In some embodiments, the reduction in animal allergens in the protein compositions described herein compared to raw animal is about 89% to about 100%. In some embodiments, the reduction in animal allergens in the protein compositions described herein compared to raw animal is about 89% to about 90%, about 89% to about 91%, about 89% to about 92%, about 89% to about 93%, about 89% to about 94%, about 89% to about 95%, about 89% to about 96%, about 89% to about 97%, about 89% to about 98%, about 89% to about 99%, about 89% to about 100%, about 90% to about 91%, about 90% to about 92%, about 90% to about 93%, about 90% to about 94%, about 90% to about 95%, about 90% to about 96%, about 90% to about 97%, about 90% to about 98%, about 90% to about 99%, about 90% to about 100%, about 91% to about 92%, about 91% to about 93%, about 91% to about 94%, about 91% to about 95%, about 91% to about 96%, about 91% to about 97%, about 91% to about 98%, about 91% to about 99%, about 91% to about 100%, about 92% to about 93%, about 92% to about 94%, about 92% to about 95%, about 92% to about 96%, about 92% to about 97%, about 92% to about 98%, about 92% to about 99%, about 92% to about 100%, about 93% to about 94%, about 93% to about 95%, about 93% to about 96%, about 93% to about 97%, about 93% to about 98%, about 93% to about 99%, about 93% to about 100%, about 94% to about 95%, about 94% to about 96%, about 94% to about 97%, about 94% to about 98%, about 94% to about 99%, about 94% to about 100%, about 95% to about 96%, about 95% to about 97%, about 95% to about 98%, about 95% to about 99%, about 95% to about 100%, about 96% to about 97%, about 96% to about 98%, about 96% to about 99%, about 96% to about 100%, about 97% to about 98%, about 97% to about 99%, about 97% to about 100%, about 98% to about 99%, about 98% to about 100%, or about 99% to about 100%. In some embodiments, the reduction in animal allergens in the protein compositions described herein compared to raw animal is about 89%, about 90%, about 91%, about 92%, about 93%, about 94%, about 95%, about 96%, about 97%, about 98%, about 99%, or about 100%. In some embodiments, the reduction in animal allergens in the protein compositions described herein compared to raw animal is at least about 89%, about 90%, about 91%, about 92%, about 93%, about 94%, about 95%, about 96%, about 97%, about 98%, or about 99%. In some embodiments, the reduction in animal allergens in the protein compositions described herein compared to raw animal is at most about 90%, about 91%, about 92%, about 93%, about 94%, about 95%, about 96%, about 97%, about 98%, about 99%, or about 100%.

In some embodiments, the reduction in parvalbumin protein in the protein compositions described herein compared to raw animal is about 89% to about 100%. In some embodiments, the reduction in parvalbumin protein in the protein compositions described herein compared to raw animal is about 89% to about 90%, about 89% to about 91%, about 89% to about 92%, about 89% to about 93%, about 89% to about 94%, about 89% to about 95%, about 89% to about 96%, about 89% to about 97%, about 89% to about 98%, about 89% to about 99%, about 89% to about 100%, about 90% to about 91%, about 90% to about 92%, about 90% to about 93%, about 90% to about 94%, about 90% to about 95%, about 90% to about 96%, about 90% to about 97%, about 90% to about 98%, about 90% to about 99%, about 90% to about 100%, about 91% to about 92%, about 91% to about 93%, about 91% to about 94%, about 91% to about 95%, about 91% to about 96%, about 91% to about 97%, about 91% to about 98%, about 91% to about 99%, about 91% to about 100%, about 92% to about 93%, about 92% to about 94%, about 92% to about 95%, about 92% to about 96%, about 92% to about 97%, about 92% to about 98%, about 92% to about 99%, about 92% to about 100%, about 93% to about 94%, about 93% to about 95%, about 93% to about 96%, about 93% to about 97%, about 93% to about 98%, about 93% to about 99%, about 93% to about 100%, about 94% to about 95%, about 94% to about 96%, about 94% to about 97%, about 94% to about 98%, about 94% to about 99%, about 94% to about 100%, about 95% to about 96%, about 95% to about 97%, about 95% to about 98%, about 95% to about 99%, about 95% to about 100%, about 96% to about 97%, about 96% to about 98%, about 96% to about 99%, about 96% to about 100%, about 97% to about 98%, about 97% to about 99%, about 97% to about 100%, about 98% to about 99%, about 98% to about 100%, or about 99% to about 100%. In some embodiments, the reduction in parvalbumin protein in the protein compositions described herein compared to raw animal is about 89%, about 90%, about 91%, about 92%, about 93%, about 94%, about 95%, about 96%, about 97%, about 98%, about 99%, or about 100%. In some embodiments, the reduction in parvalbumin protein in the protein compositions described herein compared to raw animal is at least about 89%, about 90%, about 91%, about 92%, about 93%, about 94%, about 95%, about 96%, about 97%, about 98%, or about 99%. In some embodiments, the reduction in parvalbumin protein in the protein compositions described herein compared to raw animal is at most about 90%, about 91%, about 92%, about 93%, about 94%, about 95%, about 96%, about 97%, about 98%, about 99%, or about 100%.

In some embodiments, said protein powder used in the present disclosure comprises a plant-based protein.

### Protein Isolation Methods

In various embodiments, an animal protein described herein is prepared by a process comprising sanitizing raw material from aquatic animals mixture with ozone, combining the raw material with a solvent to create a mixture, baking the combined mixture for a first time period, separating, with a filter, liquid from the combined mixture that was baked for the first time period, baking the combined mixture without the separated liquid for a second time period, separating, with a filter, liquid from the combined mixture that was baked for the second time period, curing the combined mixture, and processing the cured mixture to produce protein powder.

In some embodiments, the process further comprises baking the combined mixture without the separated liquid for a third time period and separating liquid from the combined mixture that was baked for the third time period. The process may further comprise filtering amine from the liquid separated by the filter.

The process, in some embodiments, may comprise distilling the liquid and filtering at least a portion of the distilled liquid to produce animal oil. Further, the process may comprise separating the liquid by filtering. One portion of the liquid may be distilled. The other portion may be water which may be purified.

The process may comprise adding solvent to the combined mixture prior to baking for the first time period and adding solvent to the combined mixture prior to baking for the second time period. Further, the process may comprise grinding the raw material prior to combining the raw material and the solvent.

The solvent may comprise isopropyl alcohol. In some embodiments, the combined mixture may be baked for a first time period and the mixture rotated. The process may further comprise distilling the liquid to recover the solvent.

An exemplary system for animal protein isolation comprises a preparation tank, a reactor, a filter, a mill, and an oven. The preparation tank may be configured to sanitize raw material from aquatic animals with ozone and to combine the raw material with a solvent to create a mixture. The reactor may be configured to bake the combined mixture for a first time period and bake the combined mixture of a second time period. The filter may be configured to separate solvent, oil, water, and amine from the combined mixture after baking in the reactor for the first time and configured to separate solvent, water, and amine from the combined mixture after baking in the reactor for the second time. The mill may be configured to grind the combined mixture. The oven may be configured to cure the ground combined mixture to produce protein powder.

The starting material from which protein is to be recovered includes animal tissue. Animal tissue includes eukaryotic cells of various shapes and sizes. Animal cells are further characterized as excluding cell walls which are present in all plant cells. The animal tissue may include, but is not limited to, land and marine animals such as insects, fish, poultry and red meat. In an exemplary embodiment, the starting material includes animal tissue from a piscine animal or marine animal. Suitable examples of the starting material include, but are not limited to, tissue material derived from flesh or eggs from anchovies, arctic char, mackerel, sablefish, herrings, sardines, salmon, hake (cod family), halibut, carp, trout, oysters, krill, squid, shrimp and cuttlefish, and as an optional starting raw material, dried fishmeal or dried fish, or any combinations thereof. In some embodiments, the starting material is maintained at temperatures less than 50° F., preferably less than 45° F., and more preferably less than or equal to 40° F., prior to being processed by the protein composition recovery systems provided herein. The starting material can include animal tissue from fish, and in particular, raw fish. The raw fish should be fresh and handled in a sanitary manner. The quality of the raw material should also be verified. The fish is ground, as explained above (see e.g., grinding unit 100), into pieces prior to mixing with organic solvent and further processing. The ground animal tissue is prepared by dispensing whole raw fish and/or raw fish parts to a grinding unit. The resultant ground material will contain the complete animal components inclusive of tissue, bones, and scales. The ground material is then dispensed into a suitable vessel containing an organic solvent.

A finely ground raw fish, such as those materials with a resultant particle size of less than 5000 μm, demonstrates improved filtration characteristics over an otherwise coarsely ground material, such as a material having a particle size greater than 5000 μm. The improved filtration is evident on the belt filter system where the filtration time is significantly faster for finely ground material in comparison to a ground fish material having a particle size greater than 5000 μm. An organic solvent is generally used in the process. The solvent may include an alcohol. In some embodiments, the solvent may include one or more organic solvents with a VOC ranging between about 200-500 g/L. In some embodiments, the solvent is selected such that it meets VOC regulations promulgated by local governing authority. In a preferred embodiment, the solvent includes or is IPA (isopropyl alcohol).

In some embodiments, animal meal can be used as the starting material. A mixture of animal meal and solvent is initially heated; however, a low heat, at a temperature below 75° C., is preferably used so there is no risk of decomposition, as determined by final product analysis measuring the protein content, of the protein product due to thermal degradation effects. The ratio of solvent to animal meal in the mixture should be in the range of from about 1:1 to about 2:1 so that the animal meal hydrates into a viscous liquid during processing in the slurry preparation tank, and in particular, the heating process, which is done at a controlled temperature using a variable control system that includes a temperature sensor that is immersed into the contents of the tank and that measures the actual temperature of the mixture. A corresponding temperature feedback controller measures the process temperature and regulates the amount of steam, thermal heating fluid, or electrical wattage that is used to heat the contents of the tank. The temperature controller can modulate a control valve, or wattage regulator, to control the steam, thermal heating fluid, or amperes to achieve a specified temperature setpoint, such as 70° C. to prevent the decomposition and/or denaturation of the protein material associated with the raw animal or animal meal. By preventing decomposition and/or denaturation of the protein material, the amount of protein recovered generally is greater than 85%. The ratio of animal tissue to solvent will depend on various factors including, but not limited to, the specific animal tissue and solvent used. Where the starting material is raw animal and IPA is used as the organic solvent, the ratio of raw animal in kilograms to IPA in liters ranges from about 1:1 to 1:2.2; or from about 1:1 to 1:2.1; or from about 1:1 to 1:2.0; or from about 1:1 to 1:1.9; or from about 1:1 to 1:1.8; or from about 1:1 to 1:1.7; or from about 1:1 to 1:1.6; or from about 1:1 to 1:1.5; or from about 1:1 to 1:1.4; or from about 1:1 to 1:1.3; or from about 1:1 to 1:1.2; or from about 1:1 to 1:1.1. More preferably the ratio is about 1:2. In a preferred embodiment of the present invention, upon scale-up, about 5,000 Kg of raw animal and about 10,000 L of organic solvent are combined to form the mixture of raw animal and solvent.

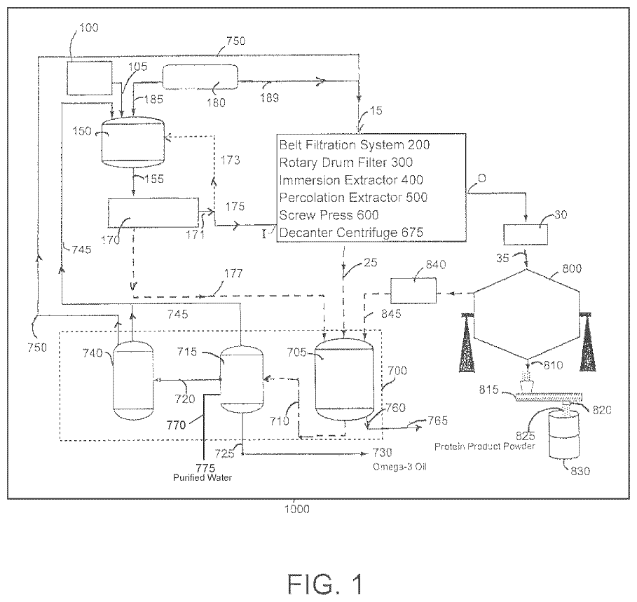

As illustrated in FIG. 1, a slurry mixture of animal tissue and organic solvent is prepared, where it is heated in the slurry tank, with agitation at a temperature ranging from about 25° C. to about 72° C. The slurry tank includes a primary agitator assembly and a heating source for providing thermal energy to the tank in order to adjust the temperature of the slurry mixture in the slurry tank. The primary agitator assembly, which consists of a rotating mixing shaft with blades, where the mixing blades are rotated by an overhead motor to achieve uniform mixing in the tank, ensures uniform mixing and heating, thus eliminating localized thermally heated zones in the tank that are in contact with the animal tissue and organic solvent mixture, particularly that portion of the mixture in proximity of the heated walls or bottom of the slurry preparation vessel. Such localized contact with thermally hot zones can induce decomposition and/or denaturing of the protein. In ensuring a thermally stable and adequately mixed environment in the slurry tank, protein conforming to the product specification will be recovered, specifically with 85% or higher protein content, as characterized by the resultant amino acid profile conducted through final product analysis.

The heating source can include a jacket encompassing at least a portion of the tank through which a thermal heating fluid, such as steam or heated oil, can be circulated or pumped; or an immersion heater that can be inserted directly into the slurry; or electrical heating elements that are in thermal communication with at least a portion of the tank; or any combination thereof. The slurry tank also can include a jacketing and insulation system to permit cooling of the tank. In some embodiment, the slurry tank includes a jacketing and insulation system that can heat and cool the tank. The slurry tank also can include a variable control system that includes a temperature sensor that is immersed into the contents of the tank and that measures the actual temperature of the mixture. The slurry tank also can include a temperature feedback controller to regulate the amount of thermal energy provided to the tank to adjust the temperature of the slurry. In some embodiments, the temperature feedback controller regulates the amount of steam, thermal heating fluid, or electrical wattage that is provided to the heating source used to heat the contents of the tank. For example, the temperature controller can modulate a control valve, or wattage regulator, or a combination thereof, to control the steam, thermal heating fluid, or amperes available to the heating source.

After processing in the slurry tank, the slurry is then transferred to a dewatering system where it undergoes dewatering to reduce the amount of water present. The dewatering system then transfers the processed slurry to the one of the 5 optional filtration extraction units.

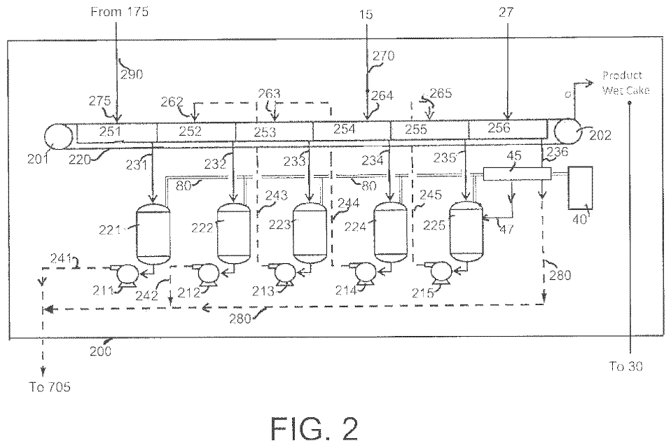

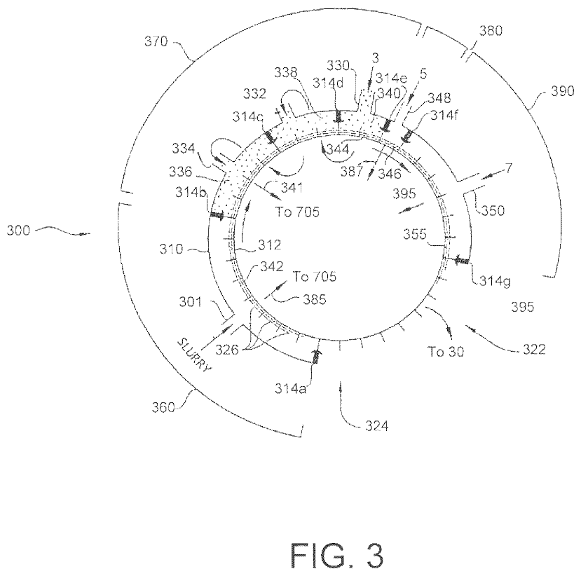

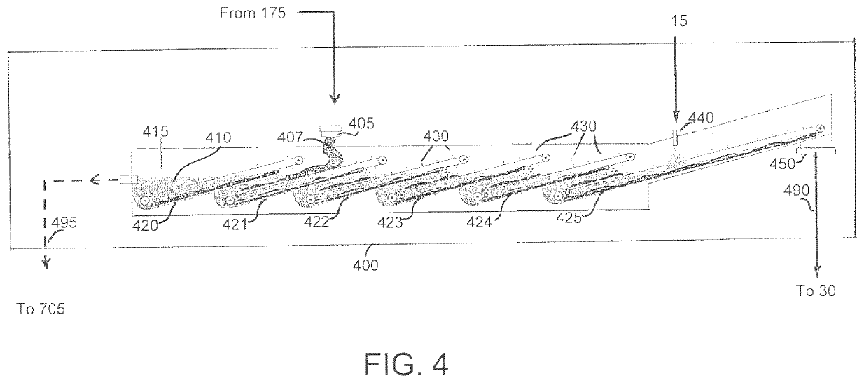

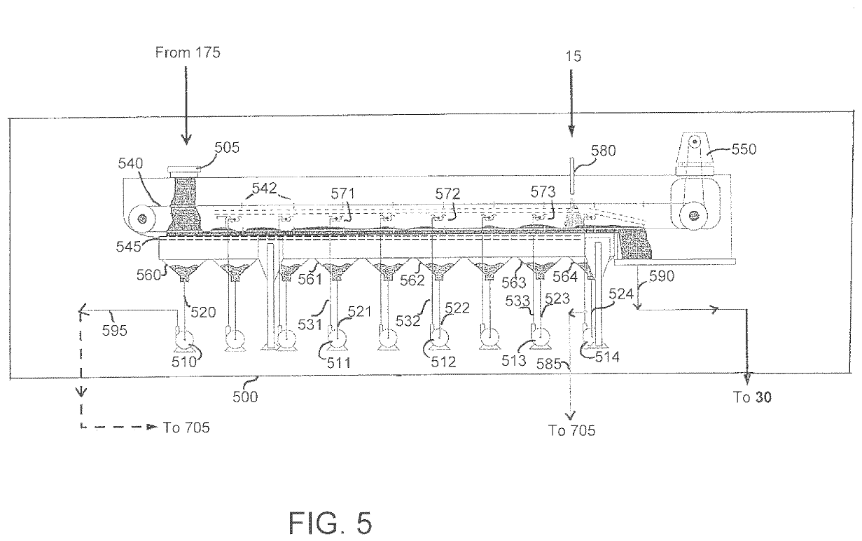

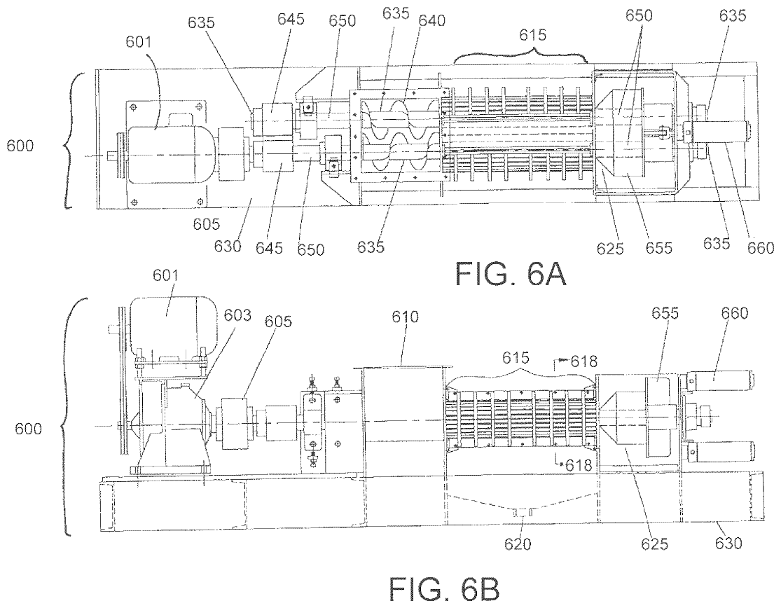

The animal tissue may be fed by the dewatering system back into the slurry preparation tank for further processing before directing the processes slurry to a filtration extraction unit. As discussed above, the slurry preparation tank can include an agitator, as well as a jacketing and insulation system to permit external heating and cooling. Preferably, the mixture is heated to a temperature not exceeding 75° C., for example, about 25-72° C. The resulting homogeneous slurry mixture is then transferred to the dewatering system and then to a filtration extraction unit of the product processing system provided herein. The product processing system, in some embodiments, includes one of a belt filtration system (an example of which is shown in FIG. 2), a rotary drum filter (an example of which is shown in FIG. 3), an immersion extractor (an example of which is shown in FIG. 4), a percolation extractor (an example of which is shown in FIG. 5, or a screw press system (an example of which is shown in FIG. 6), or any combination thereof.

In the methods provided herein, the protein material is filtered and washed. In some embodiments, a counter-current washing is used. In some embodiments, a counter-current washing system using captured filtrate is used, followed by a final fresh solvent wash. The final solvent wash can use fresh, virgin solvent, or can use solvent recovered from the system, such as by the SLR system, or a combination of the two. The recovered protein product wetcake is then dried, preferably using heat and vacuum. The nature of the continuous conveyor and filtration systems has the result of substantially increased production throughput. Continuous filtration options allow the process to be automated and to operate in closed circuit, e.g., closed system. The method also can be performed manually. In some embodiments, at least one process of the system is automated.

The low-moisture wetcake product recovered from the product processing system is further dried in a dryer and discharged from the dryer and into a particle reduction system where a micronized particle size for the product is established in the range of less than 75 μm to 250 μm. The resultant powdered protein product is then transferred to a product receptacle. The powdered protein product can be analyzed to establish whether the protein product conforms to target specifications. In an exemplary embodiment, the yield of solid protein is from about 10 wt % to about 25 wt % based upon the starting weight of the raw animal tissue starting material. Preferably, the yield of protein is in the range of from about 10 wt % to about 18 wt % based upon the starting weight of the animal tissue entering the grinding unit. In some embodiments, the yield of protein is greater than about 15 wt % based upon the starting weight of the animal tissue.

In the processes provided herein, the raw starting material generally is mixed with a food grade solvent, such as ethanol or IPA, at concentrations that can act as a disinfectant or an anti-bacterial during many stages of the process. The separated solid material containing the protein composition can be in contact with at least some amount of the food grade solvent throughout the process, making material and equipment handling easier and making maintenance of sanitary conditions throughout the process easier. This can insure that the final protein composition and omega-3 oil product obtained using the processes provided herein are sanitary and safe for human consumption.

In some embodiments, the protein compositions of the present disclosure may be derived from an animal. In some instances, the animal may comprise a chicken, fish, cow, pig, goat, sheep, or any combination thereof. In some embodiments, the protein composition may be derived from one or more chickens. In some embodiments, the protein composition may be derived from one or more cows. In some embodiments, the protein composition may be derived from one or more pigs. In some embodiments, the protein composition may be derived from one or more goats. In some embodiments, the protein composition may be derived from one or more goats. In some embodiments, the protein composition may be derived from one or more sheeps.

### Products and Packaging

The protein compositions of the present disclosure are typically in the form of a paste, but other forms such as powder (flowable or substantially flowable particulate compositions, or at least particulate compositions that can be easily scooped and measured with a spoon or similar other device) and solutions are also contemplated. The protein compositions of the disclosure are generally ready-to-use, i.e. no reconstitution is required before consumption thereof. As used herein, the term “ready-to-eat” when used to describe a food, means that after manufacture and packaging, the food product requires no additional processing, including but not limited to cooking, baking, microwaving, boiling, frying; or combination with components outside of the product's packaging.

The protein compositions of the present disclosure may be packaged and sealed in single or multi-use containers. For multi-use containers, these packages can be opened and then covered for repeated use by the ultimate user. In some embodiments, the protein compositions disclosed herein may be packaged in a squeezable pouch, tube, or packet using conventional packaging techniques. The protein compositions may be squeezed from the pouch, tube, or packet to deliver the protein composition directly into a patient's mouth.

In some embodiments, a single serving of the protein compositions is about 10 gram (g) to about 300 g. In some embodiments, a single serving of the protein compositions is at least about 10 g. In some embodiments, a single serving of the protein compositions is at most about 300 g. In some embodiments, a single serving of the protein compositions is about 10 g to about 20 g, about 10 g to about 30 g, about 10 g to about 40 g, about 10 g to about 50 g, about 10 g to about 60 g, about 10 g to about 70 g, about 10 g to about 80 g, about 10 g to about 90 g, about 10 g to about 100 g, about 10 g to about 200 g, about 10 g to about 300 g, about 20 g to about 30 g, about 20 g to about 40 g, about 20 g to about 50 g, about 20 g to about 60 g, about 20 g to about 70 g, about 20 g to about 80 g, about 20 g to about 90 g, about 20 g to about 100 g, about 20 g to about 200 g, about 20 g to about 300 g, about 30 g to about 40 g, about 30 g to about 50 g, about 30 g to about 60 g, about 30 g to about 70 g, about 30 g to about 80 g, about 30 g to about 90 g, about 30 g to about 100 g, about 30 g to about 200 g, about 30 g to about 300 g, about 40 g to about 50 g, about 40 g to about 60 g, about 40 g to about 70 g, about 40 g to about 80 g, about 40 g to about 90 g, about 40 g to about 100 g, about 40 g to about 200 g, about 40 g to about 300 g, about 50 g to about 60 g, about 50 g to about 70 g, about 50 g to about 80 g, about 50 g to about 90 g, about 50 g to about 100 g, about 50 g to about 200 g, about 50 g to about 300 g, about 60 g to about 70 g, about 60 g to about 80 g, about 60 g to about 90 g, about 60 g to about 100 g, about 60 g to about 200 g, about 60 g to about 300 g, about 70 g to about 80 g, about 70 g to about 90 g, about 70 g to about 100 g, about 70 g to about 200 g, about 70 g to about 300 g, about 80 g to about 90 g, about 80 g to about 100 g, about 80 g to about 200 g, about 80 g to about 300 g, about 90 g to about 100 g, about 90 g to about 200 g, about 90 g to about 300 g, about 100 g to about 200 g, about 100 g to about 300 g, or about 200 g to about 300 g. In some embodiments, a single serving of the protein compositions is about 10 g, about 20 g, about 30 g, about 40 g, about 50 g, about 60 g, about 70 g, about 80 g, about 90 g, about 100 g, about 200 g, or about 300 g. In some embodiments, a single serving of the protein compositions is about 50 g.

The protein compositions of the present disclosure may have any caloric density suitable for the targeted or intended patient population. In some embodiments, a single serving of the protein compositions comprises about 150 calories to about 450 calories. In some embodiments, a single serving of the protein compositions comprises at least about 150 calories. In some embodiments, a single serving of the protein compositions comprises at most about 450 calories. In some embodiments, a single serving of the protein compositions comprises about 150 calories to about 200 calories, about 150 calories to about 250 calories, about 150 calories to about 300 calories, about 150 calories to about 350 calories, about 150 calories to about 400 calories, about 150 calories to about 450 calories, about 200 calories to about 250 calories, about 200 calories to about 300 calories, about 200 calories to about 350 calories, about 200 calories to about 400 calories, about 200 calories to about 450 calories, about 250 calories to about 300 calories, about 250 calories to about 350 calories, about 250 calories to about 400 calories, about 250 calories to about 450 calories, about 300 calories to about 350 calories, about 300 calories to about 400 calories, about 300 calories to about 450 calories, about 350 calories to about 400 calories, about 350 calories to about 450 calories, or about 400 calories to about 450 calories. In some embodiments, a single serving of the protein compositions comprises about 150 calories, about 200 calories, about 250 calories, about 300 calories, about 350 calories, about 400 calories, or about 450 calories. In some embodiments, a single serving of the protein compositions comprises about 215 calories. In some embodiments, a single serving of the protein compositions comprises about 300 calories.

### Applications

Antihypertensive Peptides from Fish Sources

Naturally occurring ACE (angiotensin-1-converting enzyme) inhibitory peptides have a potential as antihypertensive components in functional foods or nutraceuticals. ACE inhibitory peptides from fish sources were identified in sardine meat, shellfish, tuna, bonito, and salmon. In some embodiments, described herein is a method of inhibiting ACE in a subject, said method comprising administering to a subject the protein composition disclosed herein.

### Antioxidant Peptides

In some embodiments, a protein composition described herein is an antioxidant protein composition. Antioxidants are known to be beneficial to human health as they may protect the body against molecules known as reactive oxygen species (ROS), which can attack membrane lipids, protein and DNA. This in turn can be a causative factor in many diseases such as cardiovascular disease, diabetes, cancer and Alzheimer's disease. Lipid oxidation can cause deterioration of food quality and a reduction in the shelf-life of a food product, while the consumption of foods containing lipid oxidation products has been linked to various diseases, including cancers, diabetes and cardiovascular disease.

The use of natural antioxidants in foodstuffs is appealing because of the potential health risk associated with synthetic antioxidants in vivo. Antioxidant peptides have been found in numerous foodstuffs including milk, wheat, potato, and fungi. Antioxidant potential of various food components is based on the ability of potential antioxidants to scavenge ROS, free radicals or prevent oxidation in model systems.

### Protein Formulations

While various embodiments of the disclosure have been shown and described herein, it will be obvious to those skilled in the art that such embodiments are provided by way of example only. Numerous variations, changes, and substitutions may occur to those skilled in the art without departing from the disclosure. It should be understood that various alternatives to the embodiments of the disclosure described herein may be employed.

Unless defined otherwise, all technical and scientific terms used herein have the same meaning as commonly understood by one of ordinary skill in the art to which the present disclosure belongs. In case of conflict, the present application including the definitions will control. Also, unless otherwise required by context, singular terms shall include pluralities and plural terms shall include the singular.

### Overview

Provided herein are novel highly nutritional protein formulations. In various embodiments, the protein formulations described herein comprise nutrients, minerals and/or vitamins necessary to sustain life. Additionally, the protein formulations of the disclosure may be highly concentrated, providing complete nutrition in a reduced volume. Additionally, in various examples, the protein formulations of the disclosure are prepared without the addition of water. As such, the protein formulations of the invention do not suffer from bacterial growth and spoilage even at warm temperatures and/or humid conditions. This results in unique and novel food preparations with a long shelf life. In some examples, the protein formulations described herein have long shelf life without the need for preservatives.

In some embodiments, the protein formulations described herein comprise greater than 20 wt % of a protein powder, for example greater than 30%, greater than 40%, greater than 50%, or greater than 60% protein powder. The protein powder used in the protein formulations of the disclosure may comprise a plant protein, an animal protein, or a mixture thereof. In some examples, the protein powder used in the protein formulations disclosed herein comprises fish protein. In various embodiments the protein powder used in the protein formulations of the disclosure is water insoluble. In some embodiments, the protein powder used in formulations of the disclosure is non-hygroscopic. In some embodiments, the protein powder used in the protein formulations of the disclosure is water insoluble and non-hygroscopic. Additionally, in some examples, the protein powder used in the protein formulations disclosed herein has a complete amino acid profile, comprising all essential amino acids. Furthermore, the protein powder used in the protein formulation of the disclosure has a high digestibility.

The protein powder of the disclosure also comprises fat. In some examples, the fat in the protein formulations disclosed herein comprises omega-3 rich fat, for example omega-3 rich fish oil.

The protein formulations described herein also comprise carbohydrates. In some cases, said carbohydrates are complex carbohydrates. In some examples, the complex carbohydrates additionally comprise fiber.

In some embodiments, about 50 g of the protein formulation disclosed herein comprises about 200 to 350 calories, for example about 250 to 350 calories or about 300 to 350 calories per serving.

In various embodiments, the protein formulations of the disclosure have low osmolality. In some embodiments, the protein formulations described herein have an osmolality between about 100 mOsm/kg and 1000 mOsm/kg, for example between 200 mOsm/kg and 1000 mOsm/kg, between 300 mOsm/kg and 1000 mOsm/kg, between 400 mOsm/kg and 1000 mOsm/kg, between 500 mOsm/kg and 1000 mOsm/kg, between 600 mOsm/kg and 1000 mOsm/kg, between 700 mOsm/kg and 1000 mOsm/kg, between 800 mOsm/kg and 1000 mOsm/kg, or between 900 mOsm/kg and 1000 mOsm/kg. In some embodiments, the osmolality of the protein formulations described herein is less than about 300 mOsm/kg, less than about 275 mOsm/kg, less than about 250 mOsm/kg, less than about 240 mOsm/kg, less than about 230 mOsm/kg, less than about 220 mOsm/kg, less than about 210 mOsm/kg, or less than about 200 mOsm/kg.

In various embodiments, the protein formulations described herein have a low glycemic index. The glycemic index (GI) is a ranking of food on a scale from 0 to 100 according to the extent to which they raise blood sugar levels after eating. Glucose for example has a GI of 100, sucrose has a GI of 65+ and isomaltulose has a GI of 32. Foods with a high GI (>50) are those which are rapidly digested and absorbed and result in marked fluctuations in blood sugar levels. Low-GI foods (<40), by virtue of their slow digestion and absorption, produce gradual rises in blood sugar and insulin levels, and have proven beneficial for health. Low GI diets have shown to improve both glucose and lipid levels in humans with diabetes (type 1 and type 2). They also have benefits in weight control because they help to control appetite and delay hunger. Low GI diets also reduce insulin levels and insulin resistance. In some embodiments, the protein formulations described herein have a GI of about 0 to about 70. In some embodiments, the protein formulations described herein have a GI of at least about 0. In some embodiments, the protein formulations described herein have a GI of at most about 70. In some embodiments, the protein formulations described herein have a GI of about 0 to about 10, about 0 to about 20, about 0 to about 30, about 0 to about 40, about 0 to about 50, about 0 to about 60, about 0 to about 70, about 10 to about 20, about 10 to about 30, about 10 to about 40, about 10 to about 50, about 10 to about 60, about 10 to about 70, about 20 to about 30, about 20 to about 40, about 20 to about 50, about 20 to about 60, about 20 to about 70, about 30 to about 40, about 30 to about 50, about 30 to about 60, about 30 to about 70, about 40 to about 50, about 40 to about 60, about 40 to about 70, about 50 to about 60, about 50 to about 70, or about 60 to about 70. In some embodiments, the protein formulations described herein have a GI of about 0, about 10, about 20, about 30, about 40, about 50, about 60, or about 70.

As used herein, the term “glycemic load” refers to the impact of a food on blood glucose. Glycemic load refers to the glycemic index of a food relative to its carbohydrate load. Thus, glycemic load is calculated as follows: (Glycemic Index/Available Carbohydrates)×100. In certain embodiments, the protein formulations disclosed herein will have a glycemic load of not more than about 12 on a 50 g basis. In certain embodiments, the protein formulations disclosed herein will have a glycemic load of not more than about 10 on a 50 g reference basis. In certain embodiments, the protein formulations disclosed herein will have a glycemic load of not more than about 8 on a 50 g reference basis. In certain embodiments, the protein formulations disclosed herein will have a glycemic load of not more than about 6 on a 50 g reference basis. In certain embodiments, the protein formulations disclosed herein will have a glycemic load of not more than about 4 on a 50 g reference basis. In certain embodiments, the protein formulations disclosed herein will have a glycemic load of not more than about 2 on a 50 g reference basis.

The protein formulations of the invention will have water activities that are sufficiently low to prevent the growth of most pathogenic and spoilage bacteria. The protein formulations of the disclosure have water activities that are less than or equal to about 0.90. In some embodiments, the protein formulations of the disclosure will a water activity less than about 0.85. In some embodiments the protein formulations will have a water activity not more than about 0.80, not more than about 0.75, or not more than about 0.70.

The Protein Digestibility-Corrected Amino Acid Score (PDCAAS) is a method for evaluation of protein quality that is described in Protein Quality Evaluation, Food and Nutrition Paper 51, Rome, Italy: FAO/WHO, 1991, p 35. To calculate a PDCAAS value, a food is analyzed for proximate and amino acid composition. A protein digestibility value is obtained from a data base or determined by the rat balance method. An amino acid score is determined as follows: Amino Acid Score=MCI of essential amino acid in 1.0 q test protein mg of essential amino acid in 1.0 g reference pattern

The PDCAAS is then calculated by multiplying the lowest amino acid score×true protein digestibility: PDCAAS=Lowest Amino Acid Score×True Digestibility

Scores above 1.00 are considered as 1.00.

In certain embodiments, the protein formulations of the disclosure have PDCAAS score greater than 0, for example greater than 0.5, greater than 0.6, greater than 0.7, greater than 0.8, greater than 0.9, greater than 0.97, or greater than 0.99. In other embodiments of the disclosure, the protein formulations have a PDCAAS score in the range of from about 0.6 to about 1.0. In other embodiments, PDCAAS score is in the range of 0.75 to 1.0. In some embodiments, the PDCAAS score is in the range of from 0.85 to 1.0. In some embodiments, the PDCAAS score is 1 (or greater than one, which is considered as 1).

As previously indicated, in various embodiments, the protein formulations disclosed herein are prepared without addition of water. As such the protein formulations disclosed herein comprise essentially no water. As such, the protein formulations of the disclosure have a long shelf-life. The term shelf-life as used herein means a period of time for which the protein formulation can be stored for, at ambient temperature, while maintaining substantially the same physical and chemical properties, e.g. taste, smell, color, and the like. In some examples, protein formulations disclosed herein have a shelf life of at least about 0.5 years to about 15 years. In some examples, protein formulations disclosed herein have a shelf life of at least about 0.5 years. In some examples, protein formulations disclosed herein have a shelf life of at most about 15 years. In some examples, protein formulations disclosed herein have a shelf life of at least about 0.5 years to about 1 year, about 0.5 years to about 1.5 years, about 0.5 years to about 2 years, about 0.5 years to about 2.5 years, about 0.5 years to about 3 years, about 0.5 years to about 2.5 years, about 0.5 years to about 4 years, about 0.5 years to about 4.5 years, about 0.5 years to about 5 years, about 0.5 years to about 10 years, about 0.5 years to about 15 years, about 1 year to about 1.5 years, about 1 year to about 2 years, about 1 year to about 2.5 years, about 1 year to about 3 years, about 1 year to about 2.5 years, about 1 year to about 4 years, about 1 year to about 4.5 years, about 1 year to about 5 years, about 1 year to about 10 years, about 1 year to about 15 years, about 1.5 years to about 2 years, about 1.5 years to about 2.5 years, about 1.5 years to about 3 years, about 1.5 years to about 2.5 years, about 1.5 years to about 4 years, about 1.5 years to about 4.5 years, about 1.5 years to about 5 years, about 1.5 years to about 10 years, about 1.5 years to about 15 years, about 2 years to about 2.5 years, about 2 years to about 3 years, about 2 years to about 2.5 years, about 2 years to about 4 years, about 2 years to about 4.5 years, about 2 years to about 5 years, about 2 years to about 10 years, about 2 years to about 15 years, about 2.5 years to about 3 years, about 2.5 years to about 2.5 years, about 2.5 years to about 4 years, about 2.5 years to about 4.5 years, about 2.5 years to about 5 years, about 2.5 years to about 10 years, about 2.5 years to about 15 years, about 3 years to about 2.5 years, about 3 years to about 4 years, about 3 years to about 4.5 years, about 3 years to about 5 years, about 3 years to about 10 years, about 3 years to about 15 years, about 2.5 years to about 4 years, about 2.5 years to about 4.5 years, about 2.5 years to about 5 years, about 2.5 years to about 10 years, about 2.5 years to about 15 years, about 4 years to about 4.5 years, about 4 years to about 5 years, about 4 years to about 10 years, about 4 years to about 15 years, about 4.5 years to about 5 years, about 4.5 years to about 10 years, about 4.5 years to about 15 years, about 5 years to about 10 years, about 5 years to about 15 years, or about 10 years to about 15 years. In some examples, protein formulations disclosed herein have a shelf life of at least about 0.5 years, about 1 year, about 1.5 years, about 2 years, about 2.5 years, about 3 years, about 2.5 years, about 4 years, about 4.5 years, about 5 years, about 10 years, or about 15 years. In some examples, protein formulations disclosed herein have a shelf life of at least about 15 years.

In some embodiments, by virtue of their extended shelf-life of the protein formulations of the present disclosure do not require refrigeration or freezing. The protein formulations are thus suitable for shipping and distribution. The protein formulations can be thus also be purchased in larger, more convenient and/or cost-effective quantities, as the quality of the protein formulations will be maintained for long periods of time.

### Protein

The protein formulations described herein comprise a protein. The amount of protein in the protein formulations will depend on the targeted application of the protein formulation. In some embodiments, the protein formulations described have a high protein content. In some examples, the protein compositions described herein comprise from about 10 wt % to about 95 wt % of protein. In some examples, the protein compositions described herein comprise from about 15 wt % to about 90 wt % of protein. In some examples, the protein formulations described herein comprise from about 20 wt % to about 80 wt % of protein. In some examples, the amount of protein in the protein formulations described herein is from about 20 wt % to about 75 wt %. In some examples, the amount of protein in the protein formulations described herein is at least about 20 wt %. In some examples, the amount of protein in the protein compositions described herein is from about 20 wt % to about 25 wt %, about 20 wt % to about 30 wt %, about 20 wt % to about 35 wt %, about 20 wt % to about 40 wt %, about 20 wt % to about 45 wt %, about 20 wt % to about 50 wt %, about 20 wt % to about 55 wt %, about 20 wt % to about 60 wt %, about 20 wt % to about 65 wt %, about 20 wt % to about 70 wt %, about 20 wt % to about 75 wt %, about 20 wt % to about 80 wt %, about 20 wt % to about 85 wt %, about 20 wt % to about 90 wt %, about 20 wt % to about 95 wt %, about 25 wt % to about 30 wt %, about 25 wt % to about 35 wt %, about 25 wt % to about 40 wt %, about 25 wt % to about 45 wt %, about 25 wt % to about 50 wt %, about 25 wt % to about 55 wt %, about 25 wt % to about 60 wt %, about 25 wt % to about 65 wt %, about 25 wt % to about 70 wt %, about 25 wt % to about 75 wt %, about 25 wt % to about 80 wt %, about 25 wt % to about 85 wt %, about 25 wt % to about 90 wt %, about 25 wt % to about 95 wt %, about 30 wt % to about 35 wt %, about 30 wt % to about 40 wt %, about 30 wt % to about 45 wt %, about 30 wt % to about 50 wt %, about 30 wt % to about 55 wt %, about 30 wt % to about 60 wt %, about 30 wt % to about 65 wt %, about 30 wt % to about 70 wt %, about 30 wt % to about 75 wt %, about 30 wt % to about 80 wt %, about 30 wt % to about 85 wt %, about 30 wt % to about 90 wt %, about 30 wt % to about 95 wt %, about 35 wt % to about 40 wt %, about 35 wt % to about 45 wt %, about 35 wt % to about 50 wt %, about 35 wt % to about 55 wt %, about 35 wt % to about 60 wt %, about 35 wt % to about 65 wt %, about 35 wt % to about 70 wt %, about 35 wt % to about 75 wt %, about 35 wt % to about 80 wt %, about 35 wt % to about 85 wt %, about 35 wt % to about 90 wt %, about 35 wt % to about 95 wt %, about 40 wt % to about 45 wt %, about 40 wt % to about 50 wt %, about 40 wt % to about 55 wt %, about 40 wt % to about 60 wt %, about 40 wt % to about 65 wt %, about 40 wt % to about 70 wt %, about 40 wt % to about 75 wt %, about 40 wt % to about 80 wt %, about 40 wt % to about 85 wt %, about 40 wt % to about 90 wt %, about 40 wt % to about 95 wt %, about 45 wt % to about 50 wt %, about 45 wt % to about 55 wt %, about 45 wt % to about 60 wt %, about 45 wt % to about 65 wt %, about 45 wt % to about 70 wt %, about 45 wt % to about 75 wt %, about 45 wt % to about 80 wt %, about 45 wt % to about 85 wt %, about 45 wt % to about 90 wt %, about 45 wt % to about 95 wt %, about 50 wt % to about 55 wt %, about 50 wt % to about 60 wt %, about 50 wt % to about 65 wt %, about 50 wt % to about 70 wt %, about 50 wt % to about 75 wt %, about 50 wt % to about 80 wt %, about 50 wt % to about 85 wt %, about 50 wt % to about 90 wt %, about 50 wt % to about 95 wt %, about 55 wt % to about 60 wt %, about 55 wt % to about 65 wt %, about 55 wt % to about 70 wt %, about 55 wt % to about 75 wt %, about 55 wt % to about 80 wt %, about 55 wt % to about 85 wt %, about 55 wt % to about 90 wt %, about 55 wt % to about 95 wt %, about 60 wt % to about 65 wt %, about 60 wt % to about 70 wt %, about 60 wt % to about 75 wt %, about 60 wt % to about 80 wt %, about 60 wt % to about 85 wt %, about 60 wt % to about 90 wt %, about 60 wt % to about 95 wt %, about 65 wt % to about 70 wt %, about 65 wt % to about 75 wt %, about 65 wt % to about 80 wt %, about 65 wt % to about 85 wt %, about 65 wt % to about 90 wt %, about 65 wt % to about 95 wt %, about 70 wt % to about 75 wt %, about 70 wt % to about 80 wt %, about 70 wt % to about 85 wt %, about 70 wt % to about 90 wt %, about 70 wt % to about 95 wt %, about 75 wt % to about 80 wt %, about 75 wt % to about 85 wt %, about 75 wt % to about 90 wt %, about 75 wt % to about 95 wt %, about 80 wt % to about 85 wt %, about 80 wt % to about 90 wt %, about 80 wt % to about 95 wt %, about 85 wt % to about 90 wt %, about 85 wt % to about 95 wt %, or about 90 wt % to about 95 wt %. In some examples, the amount of protein in the protein compositions described herein is from about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, about 90 wt %, or about 95 wt %.

In some embodiments, said protein used in the protein formulations comprises animal protein. In some instances, said animal protein is a protein from a marine animal. In some aspects, said animal protein is a protein from a fish. In some examples, the protein powder used in the protein formulations disclosed herein comprise protein from sardines, tuna, salmon, sharks, robalo, shrimp, octopus, and/or squid.

In some embodiments, said protein powder used in the present disclosure comprises a plant-based protein.

In some embodiments, the fish protein population comprises proteins from bonito, salmon, sardine, tuna, Alaskan pollack, yellowfin sole, shark, mackerel, cichlid fish, yellowstripe trevally, sardinelle, or hoki. In some embodiments, said fish protein population comprises proteins from bonito. In some embodiments, said fish protein population comprises proteins from salmon. In some embodiments, said fish protein population comprises proteins from sardine. In some embodiments, said fish protein population comprises proteins from tuna. In some embodiments, said fish protein population comprises proteins from Alaskan pollack. In some embodiments, said fish protein population comprises proteins from yellowfin sole. In some embodiments, said fish protein population comprises proteins from shark. In some embodiments, said fish protein population comprises proteins from mackerel. In some embodiments, said fish protein population comprises proteins from cichlid fish. In some embodiments, said fish protein population comprises proteins from yellowstripe trevally. In some embodiments, said fish protein population comprises proteins from sardinelle. In some embodiments, said fish protein population comprises proteins from hoki.

In some embodiments, said protein powder used in the present disclosure comprises a plant-based protein.

In some embodiments, the protein used in the protein formulations disclosed herein are water insoluble. In various embodiments, the protein in the protein formulation has a solubility of less than 50%, less than 40%, less than 30%, less than 20%, less than 15%, less than 10%, less than 7%, less than 5%, less than 3%, less than 2%, less than 1%, or less than 0.5% at a pH range of about 1-8, for example at a pH of 7. In some embodiments, the protein in the protein formulations disclosed herein are non-hygroscopic i.e. they do not adsorb moisture from the surrounding atmosphere.

In various embodiments, the protein used in the protein formulations disclosed herein comprises one or more essential amino acids, i.e. one or more amino acids selected from the group consisting of phenylalanine, valine, threonine, tryptophan, methionine, leucine, isoleucine, lysine, and histidine. In some embodiments, the protein used in the protein formulations disclosed herein comprises all essential amino acids. In some embodiments, the protein used in the protein formulations disclosed herein further comprises one or more non-essential amino acids, for example one or more of cysteine, aspartic acid, serine, glutamic acid, proline, glycine, alanine, tyrosine, and arginine. In some embodiments, the protein used in the protein formulations disclosed herein comprises all of phenylalanine, valine, threonine, tryptophan, methionine, leucine, isoleucine, lysine, histidine, cysteine, aspartic acid, serine, glutamic acid, proline, glycine, alanine, tyrosine, and arginine.

In some embodiments, the protein used in the protein formulations disclosed herein comprises less than 1 ppm of heavy metals, for example, less than 0.9 ppm, less than 0.8 ppm, less than 0.7 ppm, less than 0.6 ppm, less than 0.5 ppm, less than 0.4 ppm, less than 0.3 ppm, less than 0.2 ppm, less than 0.1 ppm or less than 0.05 ppm of heavy metals.

In some embodiments, the protein used in the protein formulations disclosed herein is non-hydrolyzed. In some examples, the protein in the protein formulations disclosed herein has a degree of hydrolysis of about 1% to about 50%. In some examples, the protein in the protein formulations disclosed herein has a degree of hydrolysis of at least about 1%. In some examples, the protein in the protein formulations disclosed herein has a degree of hydrolysis of at most about 50%. In some examples, the protein in the protein formulations disclosed herein has a degree of hydrolysis of about 1% to about 2%, about 1% to about 4%, about 1% to about 6%, about 1% to about 8%, about 1% to about 10%, about 1% to about 15%, about 1% to about 20%, about 1% to about 25%, about 1% to about 30%, about 1% to about 40%, about 1% to about 50%, about 2% to about 4%, about 2% to about 6%, about 2% to about 8%, about 2% to about 10%, about 2% to about 15%, about 2% to about 20%, about 2% to about 25%, about 2% to about 30%, about 2% to about 40%, about 2% to about 50%, about 4% to about 6%, about 4% to about 8%, about 4% to about 10%, about 4% to about 15%, about 4% to about 20%, about 4% to about 25%, about 4% to about 30%, about 4% to about 40%, about 4% to about 50%, about 6% to about 8%, about 6% to about 10%, about 6% to about 15%, about 6% to about 20%, about 6% to about 25%, about 6% to about 30%, about 6% to about 40%, about 6% to about 50%, about 8% to about 10%, about 8% to about 15%, about 8% to about 20%, about 8% to about 25%, about 8% to about 30%, about 8% to about 40%, about 8% to about 50%, about 10% to about 15%, about 10% to about 20%, about 10% to about 25%, about 10% to about 30%, about 10% to about 40%, about 10% to about 50%, about 15% to about 20%, about 15% to about 25%, about 15% to about 30%, about 15% to about 40%, about 15% to about 50%, about 20% to about 25%, about 20% to about 30%, about 20% to about 40%, about 20% to about 50%, about 25% to about 30%, about 25% to about 40%, about 25% to about 50%, about 30% to about 40%, about 30% to about 50%, or about 40% to about 50%. In some examples, the protein in the protein formulations disclosed herein has a degree of hydrolysis of about 1%, about 2%, about 4%, about 6%, about 8%, about 10%, about 15%, about 20%, about 25%, about 30%, about 40%, or about 50%.

The proteins in the protein formulations described herein have good digestibility. In some embodiments, the protein in the protein formulation has a digestibility coefficient of at least about 70%. In some embodiments, the protein in the protein formulation has a digestibility coefficient of at least about 80%. In some embodiments, the protein in the protein formulation has a digestibility coefficient of at least about 90%. In some embodiments, the protein in the protein formulation has a digestibility coefficient of at least about 95%. In some embodiments, the protein in the protein formulation has a digestibility coefficient of at least about 97%. In some embodiments, the protein in the protein formulation has a digestibility coefficient of at least about 98%. In some embodiments, the protein in the protein formulation has a digestibility coefficient of at least about 99%.

The proteins in the protein formulations described herein have good digestibility. In some embodiments, the protein in the protein formulation has a digestibility coefficient greater than about 70%. In some embodiments, the protein in the protein formulation has a digestibility coefficient of greater than about 80%. In some embodiments, the protein in the protein formulation has a digestibility coefficient of greater than about 90%. In some embodiments, the protein in the protein formulation has a digestibility coefficient of at greater than about 95%. In some embodiments, the protein in the protein formulation has a digestibility coefficient of greater than about 97%. In some embodiments, the protein in the protein formulation has a digestibility coefficient of greater than about 98%. In some embodiments, the protein in the protein formulation has a digestibility coefficient of greater than about 99%.

### Fat

In various embodiments, the protein formulations disclosed herein also comprise fat. The fat content of the protein formulations is adjusted depending on the use. In some cases, higher fat compositions can be prepared. In other instances, lower fat compositions are desired. Any edible fat can be used in the protein formulations described herein.

In some embodiments, the fat is added to the protein formulations in the form of an oil. Oil may also provide a source of calories. The oil in the protein formulations may additionally improve the texture and/or mouthfeel of the protein formulations. Further, the oil may also aid in the functionality of the protein formulations. Non limiting oils that can be used in the protein formulations described herein include fish oil, palm oil, coconut oil, rapeseed oil, sunflower oil, peanut oil, cottonseed oil, palm kernel oil, olive oil, corn oil, hazelnut oil, linseed oil, rice bran oil, sesame oil, safflower oil, canola oil, flax seed oil, soybean oil, blubber, cod liver oil, mustard oil, walnut oil, lard pork fat, beef tallow, chicken fat, chia seed oil, and combinations thereof. In some embodiments, the oils used in the protein formulations described herein are omega-3 rich oils, for example canola oil, fish oil, flaxseed oil, mustard oil, soybean oil, and walnut oil. In some embodiments, the oils used in the protein formulations described herein is fish oil, for example omega-3 rich fish oil. In some embodiments, the fish oil used in the protein formulations described herein has a high omega-3 oil content, such as 50% or higher, 60% or higher, 70% or higher, 80% or higher, 90% or higher, or 95% or higher. Suitable fish oils include, but are not limited to, pilchard oil, menhaden oil, Peruvian fish oil, sardine oil, salmon oil, herring oil, and mackerel oil.

The oils used in the protein formulations described herein may be unsaturated, partially hydrogenated, or hydrogenated. In some embodiments, the amount of trans-fat in the protein formulations described herein is low. For example, the amount of trans fat in the protein formulations described herein may be less than 20%, less than 15%, less than 10%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, or less than 0.05%.

### System for Producing the Protein and Fat

The system and methods described herein for the processes of producing protein and fat are described in U.S. Pat. No. 9,826,757, which is incorporated by reference herein in its entirety.

### Carbohydrates

In various embodiments, the protein formulations described herein also comprise carbohydrate. In various embodiments, the carbohydrates in the protein formulations may provide calories and/or act as a bulking agent. In some embodiments, the carbohydrates can also provide a smooth mouthfeel to the composition. The carbohydrate content of the protein formulation can be from about 5 wt. % to about 95 wt. % based on the total weight of the composition. In some embodiments, the carbohydrate content of the protein formulation can be from about 10 wt % to about 90 wt %. In some embodiments, the carbohydrate content of the protein formulation can be from at least about 10 wt %. In various embodiments, the carbohydrate composition can be from at most about 90 wt %. In various embodiments, the carbohydrate composition can be from about 10 wt % to about 15 wt %, about 10 wt % to about 25 wt %, about 10 wt % to about 30 wt %, about 10 wt % to about 35 wt %, about 10 wt % to about 40 wt %, about 10 wt % to about 45 wt %, about 10 wt % to about 50 wt %, about 10 wt % to about 60 wt %, about 10 wt % to about 70 wt %, about 10 wt % to about 80 wt %, about 10 wt % to about 90 wt %, about 15 wt % to about 25 wt %, about 15 wt % to about 30 wt %, about 15 wt % to about 35 wt %, about 15 wt % to about 40 wt %, about 15 wt % to about 45 wt %, about 15 wt % to about 50 wt %, about 15 wt % to about 60 wt %, about 15 wt % to about 70 wt %, about 15 wt % to about 80 wt %, about 15 wt % to about 90 wt %, about 25 wt % to about 30 wt %, about 25 wt % to about 35 wt %, about 25 wt % to about 40 wt %, about 25 wt % to about 45 wt %, about 25 wt % to about 50 wt %, about 25 wt % to about 60 wt %, about 25 wt % to about 70 wt %, about 25 wt % to about 80 wt %, about 25 wt % to about 90 wt %, about 30 wt % to about 35 wt %, about 30 wt % to about 40 wt %, about 30 wt % to about 45 wt %, about 30 wt % to about 50 wt %, about 30 wt % to about 60 wt %, about 30 wt % to about 70 wt %, about 30 wt % to about 80 wt %, about 30 wt % to about 90 wt %, about 35 wt % to about 40 wt %, about 35 wt % to about 45 wt %, about 35 wt % to about 50 wt %, about 35 wt % to about 60 wt %, about 35 wt % to about 70 wt %, about 35 wt % to about 80 wt %, about 35 wt % to about 90 wt %, about 40 wt % to about 45 wt %, about 40 wt % to about 50 wt %, about 40 wt % to about 60 wt %, about 40 wt % to about 70 wt %, about 40 wt % to about 80 wt %, about 40 wt % to about 90 wt %, about 45 wt % to about 50 wt %, about 45 wt % to about 60 wt %, about 45 wt % to about 70 wt %, about 45 wt % to about 80 wt %, about 45 wt % to about 90 wt %, about 50 wt % to about 60 wt %, about 50 wt % to about 70 wt %, about 50 wt % to about 80 wt %, about 50 wt % to about 90 wt %, about 60 wt % to about 70 wt %, about 60 wt % to about 80 wt %, about 60 wt % to about 90 wt %, about 70 wt % to about 80 wt %, about 70 wt % to about 90 wt %, or about 80 wt % to about 90 wt %. In various embodiments, the carbohydrate composition can be from about 10 wt %, about 15 wt %, about 25 wt %, about 30 wt %, about 35 wt %, about 40 wt %, about 45 wt %, about 50 wt %, about 60 wt %, about 70 wt %, about 80 wt %, or about 90 wt %.

Non limiting examples of the carbohydrates that can be used in the protein formulations described herein include rye carbohydrates, barley carbohydrates, corn carbohydrates, cassava carbohydrates, oat carbohydrates, rice carbohydrates, millet carbohydrates, sorghum carbohydrates, tapioca carbohydrates, or a combination thereof. In some examples, the carbohydrates can be selected from corn, nuts, barley, bulger, pasta, parboiled rice, dried legumes, or mixtures thereof. In some embodiments, the carbohydrates comprise barley carbohydrates.

In some embodiments, the carbohydrates in the protein formulations comprise complex carbohydrates. For example, greater than 10%, 15%, 20%, 30%, 40%, 50%, 60%, 70%, 80%, 90%, 95%, 97%, 98%, 99%, or 99.5% of the carbohydrates in the protein formulations are complex carbohydrates. In some embodiments, the carbohydrates in the protein formulations also comprise fiber. In some embodiments, the protein formulations comprise complex carbohydrates from barley and optionally include fiber.

### Vitamins

In various embodiments, the protein formulation of the present disclosure may further comprise one or more vitamins. Non limiting examples, of the vitamins that may be present in the protein formulations described herein include vitamin A (including retinol, retinal and retinoic acid, as well as provitamin carotenoids such as beta-carotene), vitamin B (including thiamin (vitamin B1), riboflavin (vitamin B2), niacin (vitamin B3), pantothenic acid (vitamin B5), pyridoxine (vitamin B6), biotin (vitamin B7), folate (vitamin B9), and cobalamin (vitamin B12), vitamin C (ascorbic acid), vitamin D (including vitamin D3 (cholecalciferol) and vitamin D2 (ergocalciferol)), vitamin E (including alpha-tocopherol), and vitamin K (including vitamin K₁ or phylloquinone, vitamin K2, and vitamin K3 or menadione).

In some embodiments, the protein formulations comprise all of vitamin A, vitamin B1, vitamin B2, vitamin B3, vitamin B5, vitamin B6, vitamin B7, vitamin B9, vitamin B12, vitamin C, vitamin D, vitamin E, and vitamin K.

In some embodiments, the one or more vitamins may be present in nutritionally relevant amounts, which means that a serving of protein formulations provide a nourishing amount of said vitamins. In some embodiments, this amount comprises at least about 1% of the U.S. RDA (Recommended Dietary Allowances) or RDI (Reference Daily Intake) for these vitamins. In some embodiments, this amount comprises from about 1% of the U.S. RDA or RDI to about 100% of the U.S. RDA or RDI. In some embodiments, this amount comprises from about 10% to about 100% of the U.S. RDA or RDI, about 20% to about 100% of the U.S. RDA or RDI, about 30% to about 100% of the U.S. RDA or RDI, about 40% to about 100% of the U.S. RDA or RDI, about 50% to about 100% of the U.S. RDA or RDI, about 60% to about 100% of the U.S. RDA or RDI, about 70% to about 100% of the U.S. RDA or RDI, about 80% to about 100% of the U.S. RDA or RDI, about 90% to about 100% of the U.S. RDA or RDI, about 92% to about 100% of the U.S. RDA or RDI, about 94% to about 100% of the U.S. RDA or RDI, about 96% to about 100% of the U.S. RDA or RDI, about 98% to about 100% of the U.S. RDA or RDI, or about 99% to about 100% of the U.S. RDA or RDI. Of course, it is recognized that the preferred daily intake of any vitamin may vary with the user, with greater than the U.S. RDA or RDI intakes being beneficial in some circumstances.

### Emulsifiers

In some embodiments, the protein formulations of the present disclosure further comprise an emulsifier. In some instances, an emulsifying agent aids in binding the fat and minimizing oil separation in the protein formulation. The emulsifying agent can be the product of an esterification of a diglyceride and glycerol, a lecithin, a monoglyceride or a diglyceride of a fatty acid ester, a monoglyceride of a fatty acid, or a phosphated monoglyceride. The diglyceride source for the product of an esterification of a diglyceride and glycerol can be a palm oil, a rapeseed oil, a soybean oil, a sunflower oil, a lard, a tallow, or a combination thereof. In various preferred embodiments, the emulsifying agent is a Dimodan® HS-KA, Dimodan® Visco-Lo, Dimodan® SO or sodium stearoyl lactylate, or a combination thereof.

Typically, the emulsifying agent is added to the food composition in an amount of 0.3 wt. % to about 1.5 wt. % based on the total weight of the composition; preferably, the emulsifying agent is added to the food composition in an amount of about 0.8 wt. % based on the total weight of the composition.

### Electrolytes

In various embodiments, the protein formulations of the present disclosure further comprises electrolytes. Non limiting examples of electrolytes that can be present in the protein formulations disclosed here in include calcium, chloride, fluoride, iodine, magnesium, zinc, chromium, potassium, sodium, sulfate, hydrosulfate, carbonate, hydrocarbonate, and phosphates.

### Minerals

In various embodiments, the protein formulations of the present disclosure further comprises one or more minerals. Non limiting examples of the minerals that may be present in the instantly disclosed protein formulations include calcium, chloride, chromium, copper, fluoride, iodine, iron, magnesium, manganese, molybdenum, phosphorus, potassium, selenium, sodium, sulfur, and zinc. In some embodiments, the mineral sources are may be present in nutritionally relevant amounts, which means that a serving of protein formulations provide a nourishing amount of said minerals. In some embodiments, this amount comprises at least about 1% of the U.S. RDA (Recommended Dietary Allowances) or RDI (Reference Daily Intake) for these minerals. In some embodiments, this amount comprises from about 1% of the U.S. RDA or RDI to about 100% of the U.S. RDA or RDI. In some embodiments, this amount comprises from about 10% to about 100% of the U.S. RDA or RDI, about 20% to about 100% of the U.S. RDA or RDI, about 30% to about 100% of the U.S. RDA or RDI, about 40% to about 100% of the U.S. RDA or RDI, about 50% to about 100% of the U.S. RDA or RDI, about 60% to about 100% of the U.S. RDA or RDI, about 70% to about 100% of the U.S. RDA or RDI, about 80% to about 100% of the U.S. RDA or RDI, about 90% to about 100% of the U.S. RDA or RDI, about 92% to about 100% of the U.S. RDA or RDI, about 94% to about 100% of the U.S. RDA or RDI, about 96% to about 100% of the U.S. RDA or RDI, about 98% to about 100% of the U.S. RDA or RDI, or about 99% to about 100% of the U.S. RDA or RDI. Of course, it is recognized that the preferred daily intake of any mineral may vary with the user, with greater than the U.S. RDA or RDI intakes being beneficial in some circumstances.

### Flavoring Agents

In some embodiments, the protein formulation of the present disclosure further comprises one of more flavoring agents.

In various embodiments the protein formulations disclosed herein further comprise one or more sweeteners. The sweeteners suitable for the protein formulation disclosed herein include any edible sweetener suitable for use in food or beverage, or an ingredient of a food or beverage, for human or animal consumption.

Sweeteners suitable for use in various embodiments of the protein formulations disclosed here include nutritive and non-nutritive, natural, and artificial or synthetic sweeteners. Suitable sweetener is selected based on the desired nutritional characteristics, taste profile, mouthfeel, and other organoleptic factors. Non-nutritive artificial sweeteners suitable for the protein formulations include, for example, peptide based sweeteners, e.g., aspartame, neotame, and alitame, and non-peptide based sweeteners, for example, sodium saccharin, calcium saccharin, acesulfame potassium, sodium cyclamate, calcium cyclamate, neohesperidin dihydrochalcone, and sucralose.

Other non-nutritive sweeteners that can be used include, for example, sorbitol, mannitol. xylitol, glycyrrhizin, D-tagatose, erythritol, meso-erythritol, maltitol, maltose, lactose, fructo-oligosaccharides, Lo Han Guo powder, steviol glycosides, e.g., rebaudiosides such as rebaudioside A, stevioside, etc., acesulfame, aspartame, other dipeptides, cyclamate, sucralose, saccharin, xylose, arabinose, isomalt, lactitol, maltitol, trehalose, and ribose, and protein sweeteners such as thaumatin, monellin, brazzein, L-alanine and glycine, related compounds, and mixtures of any of them.

In some embodiments, the protein formulations of the disclosure can include nutritive, natural crystalline or liquid sweeteners such as sucrose, liquid sucrose, fructose, liquid fructose, glucose, liquid glucose, glucose-fructose syrup from natural sources (such as apple, chicory, honey, etc.), high fructose corn syrup, invert sugar, maple syrup, maple sugar, honey, brown sugar molasses (e.g., cane molasses. Such as first molasses, second molasses, blackstrap molasses, and Sugar beet molasses), sorghum syrup, Lo Han Guo juice concentrate and/or others.

The total amount of sweeteners in the protein formulation will depend on several factors, for example upon the desired level of sweetness for the protein formulation. In some examples, the total amount of sweeteners present in the protein formulation is in the range of from about 0.1 wt % to about 20 wt % based on the total weight of the protein formulation. In some embodiments, the amount of sweetener in the protein formulation is at least about 0.1 wt %. In some embodiments, the amount of sweetener in the protein formulation is at most about 20 wt %. In some embodiments, the amount of sweetener in the protein formulation is about 0.1 wt % to about 1 wt %, about 0.1 wt % to about 2 wt %, about 0.1 wt % to about 4 wt %, about 0.1 wt % to about 6 wt %, about 0.1 wt % to about 8 wt %, about 0.1 wt % to about 10 wt %, about 0.1 wt % to about 12 wt %, about 0.1 wt % to about 14 wt %, about 0.1 wt % to about 16 wt %, about 0.1 wt % to about 18 wt %, about 0.1 wt % to about 20 wt %, about 1 wt % to about 2 wt %, about 1 wt % to about 4 wt %, about 1 wt % to about 6 wt %, about 1 wt % to about 8 wt %, about 1 wt % to about 10 wt %, about 1 wt % to about 12 wt %, about 1 wt % to about 14 wt %, about 1 wt % to about 16 wt %, about 1 wt % to about 18 wt %, about 1 wt % to about 20 wt %, about 2 wt % to about 4 wt %, about 2 wt % to about 6 wt %, about 2 wt % to about 8 wt %, about 2 wt % to about 10 wt %, about 2 wt % to about 12 wt %, about 2 wt % to about 14 wt %, about 2 wt % to about 16 wt %, about 2 wt % to about 18 wt %, about 2 wt % to about 20 wt %, about 4 wt % to about 6 wt %, about 4 wt % to about 8 wt %, about 4 wt % to about 10 wt %, about 4 wt % to about 12 wt %, about 4 wt % to about 14 wt %, about 4 wt % to about 16 wt %, about 4 wt % to about 18 wt %, about 4 wt % to about 20 wt %, about 6 wt % to about 8 wt %, about 6 wt % to about 10 wt %, about 6 wt % to about 12 wt %, about 6 wt % to about 14 wt %, about 6 wt % to about 16 wt %, about 6 wt % to about 18 wt %, about 6 wt % to about 20 wt %, about 8 wt % to about 10 wt %, about 8 wt % to about 12 wt %, about 8 wt % to about 14 wt %, about 8 wt % to about 16 wt %, about 8 wt % to about 18 wt %, about 8 wt % to about 20 wt %, about 10 wt % to about 12 wt %, about 10 wt % to about 14 wt %, about 10 wt % to about 16 wt %, about 10 wt % to about 18 wt %, about 10 wt % to about 20 wt %, about 12 wt % to about 14 wt %, about 12 wt % to about 16 wt %, about 12 wt % to about 18 wt %, about 12 wt % to about 20 wt %, about 14 wt % to about 16 wt %, about 14 wt % to about 18 wt %, about 14 wt % to about 20 wt %, about 16 wt % to about 18 wt %, about 16 wt % to about 20 wt %, or about 18 wt % to about 20 wt %. In some embodiments, the amount of sweetener in the protein formulation is from about 0.1 wt %, about 1 wt %, about 2 wt %, about 4 wt %, about 6 wt %, about 8 wt %, about 10 wt %, about 12 wt %, about 14 wt %, about 16 wt %, about 18 wt %, or about 20 wt %.

### Stabilizing Agents

In various embodiments, the protein formulations disclosed herein further comprises one or more stabilizing agents. Non limiting examples of the stabilizing agents that can be used in the instant protein formulations include alginate, agar, carrageen, cellulose and cellulose derivatives, gelatin, guar gum, gum Arabic, locust bean gum, pectin, starch, xanthan gum, sterins, tocopherols, and ascorbyl palmitate. In some embodiments, the stabilizing agent is sterins, tocopherols, or ascorbyl palmitate.

### Therapeutic Agents

The protein formulations of the present disclosure may additionally comprise a therapeutic agent. In some instances, the therapeutic agent may be a plant extract, for example comprises a cannabis extract. The protein formulations described herein can include extracts derived from plant materials of any plant species belonging to the genus cannabis. Non-limiting examples of suitable cannabis species include Cannabis sativa, cannabis indica, and Cannabis ruderalis. Hybrid cannabis strains and inbred cannabis strains are both suitable. Accordingly, in some embodiments, the protein formulations disclosed herein can include extracts from a hybrid cannabis strain. In some embodiments, the protein formulations can include extracts from an inbred cannabis strain. In some embodiments, protein formulations can include extracts from plant materials of one or more varieties of whole cannabis plants, particularly Cannabis sativa, cannabis indica, Cannabis ruderalis, or plants which are the result of genetic crosses, self-crosses or hybrids thereof. Non-limiting examples of suitable strains of cannabis include, blueberry, white widow, charlotte's web, diesel, haze, BC bud, Holland's hope, kush, northern lights, purple, Jack Herer, Acapulco Gold and Malawi Gold (Chamba), Shaman, Sour, Skunk, and Te Puke Thunder. Further examples of suitable cannabis strains include, but are not limited to Blackberry Kush, Blue Dream, Bubba Kush, Cherry Pie, Durban Poison, Fire OG, Girl Scout Cookies, Gorilla Glue, Grape Ape, Green Crack, Headband, Kosher Kush, Master Kush, OG Kush, Purple Haze, Purple Kush, Skywalker OG, Sour Diesel, Super Lemon Haze, Super Silver Haze, and White Widow. Additional non-limiting examples of Cannabis strains include strains that have been deposited under NCIMB Nos. 41541, 42254, 42255, 42256, 42257, and 42258. In some embodiments, the cannabis-based formulations can include extracts from any physical part of the plant materials. Suitable plant materials include, but are not limited to, e.g., the leaf, bud, flower, trichome, seed, or a combination thereof.

The Cannabis plant material contains suitable and desirable compounds, including but not limited to cannabinoids, terpenes, terpenoids, and flavonoids. Accordingly, in some embodiments, the protein formulations can include one or more cannabis-derived cannabinoid compounds. Cannabinoids can be in acid forms or in neutral forms (e.g., decarboxylated forms).

Typical cannabinoids isolated from Cannabis plants include, but are not limited to, tetrahydrocannabinol (THC), cannabidiol (CBD) and cannabinol (CBN). Other cannabinoids include for example, cannabichromene (CBC), cannabigerol (CBG) cannabinodiol (CBND), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM). As used herein THC, CBD, CBN, CBC, CBG, CBND, CBL, CBV, THCV, CBDV, CBCV, CBGV and CBGM refer to the decarboxylated form of the cannabinoid. Whereas, THCA, CBD A, CBNA, CBC A, CBGA, CBND A, CBL A, CBVA, THCVA, CBDVA, CBCVA, CBGVA and CBGAM (cannabigerolic acid mononiethyl ether) refer to the corresponding acid form of the cannabinoid. In the Cannabis plant, cannabinoids are synthesized and accumulated as cannabinoid acids (e.g., cannabidiolic acid (CBDA)). When the herbal product is dried, stored, or heated, the acids are decarboxylated gradually or completely into neutral forms (e.g., THCA→THC and CBDA→CBD).

In some embodiments, the protein formulations described herein can include one, at least two, at least three, at least four, at least five, at least six, at least seven, at least eight, at least nine, at least ten, at least eleven, or at least twelve cannabis-derived cannabinoid compounds. In some embodiments, the one or more cannabis-derived cannabinoid compounds is selected from the group consisting of THC, CBD, CBN, CBC, CBG, CBND, CBL, CBV, THCV, CBDV, CBCV, CBGV, CBGM, THCA, CBDA, CBNA, CBC A, CBGA, CBND A, CBLA, CBVA, THCVA, CBDVA, CBCVA, CBGVA, CBGAM, and combinations of any thereof. In some embodiments, protein formulations can include THC (or THCA or an active analogue thereof). In some embodiments, the protein formulations can include CBD (or CBDA or an active analogue thereof). In some embodiments, the methods and compositions as described herein can exclude one or more of the cannabis-derived cannabinoid compounds described above.

### Additional Ingredients

Additional ingredients, as needed or desired, may be incorporated in the protein formulations disclosure. Such ingredients include bracers, flavanols, coloring agents, preservatives, acidulants, and/or food stability anti-oxidants.

If desired, coloring agents can also be added to the protein formulations of the present disclosure. Any soluble coloring agents approved for food use can be utilized for the present disclosure.

When desired preservatives, such as ascorbic acid, benzoic acid, hexametaphosphate, and salts thereof can be added into the embodiments of the present disclosure.

Organic or inorganic edible acids may be used to adjust the pH of the protein formulations disclosed herein. Exemplary edible acids include citric acid, malic acid, fumaric acid, adipic acid, phosphoric acid, gluconic acid, tartaric acid, and ascorbic acid.

### Products/Packaging

The protein formulations of the present disclosure are typically in the form of a paste, but other forms such as powder (flowable or substantially flowable particulate compositions, or at least particulate compositions that can be easily scooped and measured with a spoon or similar other device) and solutions are also contemplated. The protein formulations of the disclosure are generally ready-to-use, i.e. no reconstitution is required before consumption thereof. As used herein, the term “ready-to-eat” when used to describe a food, means that after manufacture and packaging, the food product requires no additional processing, including but not limited to cooking, baking, microwaving, boiling, frying; or combination with components outside of the product's packaging.

The protein formulations of the present disclosure may be packaged and sealed in single or multi-use containers. For multi-use containers, these packages can be opened and then covered for repeated use by the ultimate user. In some embodiments, the protein formulations disclosed herein may be packaged in a squeezable pouch, tube, or packet using conventional packaging techniques. The protein formulations may be squeezed from the pouch, tube, or packet to deliver the protein formulation directly into an individual's mouth.

In some embodiments, a single serving of the protein formulations is about 10 gram (g) to about 300 g. In some embodiments, a single serving of the protein formulations is at least about 10 g. In some embodiments, a single serving of the protein formulations is at most about 300 g. In some embodiments, a single serving of the protein formulations is about 10 g to about 20 g, about 10 g to about 30 g, about 10 g to about 40 g, about 10 g to about 50 g, about 10 g to about 60 g, about 10 g to about 70 g, about 10 g to about 80 g, about 10 g to about 90 g, about 10 g to about 100 g, about 10 g to about 200 g, about 10 g to about 300 g, about 20 g to about 30 g, about 20 g to about 40 g, about 20 g to about 50 g, about 20 g to about 60 g, about 20 g to about 70 g, about 20 g to about 80 g, about 20 g to about 90 g, about 20 g to about 100 g, about 20 g to about 200 g, about 20 g to about 300 g, about 30 g to about 40 g, about 30 g to about 50 g, about 30 g to about 60 g, about 30 g to about 70 g, about 30 g to about 80 g, about 30 g to about 90 g, about 30 g to about 100 g, about 30 g to about 200 g, about 30 g to about 300 g, about 40 g to about 50 g, about 40 g to about 60 g, about 40 g to about 70 g, about 40 g to about 80 g, about 40 g to about 90 g, about 40 g to about 100 g, about 40 g to about 200 g, about 40 g to about 300 g, about 50 g to about 60 g, about 50 g to about 70 g, about 50 g to about 80 g, about 50 g to about 90 g, about 50 g to about 100 g, about 50 g to about 200 g, about 50 g to about 300 g, about 60 g to about 70 g, about 60 g to about 80 g, about 60 g to about 90 g, about 60 g to about 100 g, about 60 g to about 200 g, about 60 g to about 300 g, about 70 g to about 80 g, about 70 g to about 90 g, about 70 g to about 100 g, about 70 g to about 200 g, about 70 g to about 300 g, about 80 g to about 90 g, about 80 g to about 100 g, about 80 g to about 200 g, about 80 g to about 300 g, about 90 g to about 100 g, about 90 g to about 200 g, about 90 g to about 300 g, about 100 g to about 200 g, about 100 g to about 300 g, or about 200 g to about 300 g. In some embodiments, a single serving of the protein formulations is about 10 g, about 20 g, about 30 g, about 40 g, about 50 g, about 60 g, about 70 g, about 80 g, about 90 g, about 100 g, about 200 g, or about 300 g. In some embodiments, a single serving of the protein formulations is about 50 g.

The protein formulations of the present disclosure may have any caloric density suitable for the targeted or intended patient population. In some embodiments, a single serving of the protein formulations comprises about 150 calories to about 450 calories. In some embodiments, a single serving of the protein formulations comprises at least about 150 calories. In some embodiments, a single serving of the protein formulations comprises at most about 450 calories. In some embodiments, a single serving of the protein formulations comprises about 150 calories to about 200 calories, about 150 calories to about 250 calories, about 150 calories to about 300 calories, about 150 calories to about 350 calories, about 150 calories to about 400 calories, about 150 calories to about 450 calories, about 200 calories to about 250 calories, about 200 calories to about 300 calories, about 200 calories to about 350 calories, about 200 calories to about 400 calories, about 200 calories to about 450 calories, about 250 calories to about 300 calories, about 250 calories to about 350 calories, about 250 calories to about 400 calories, about 250 calories to about 450 calories, about 300 calories to about 350 calories, about 300 calories to about 400 calories, about 300 calories to about 450 calories, about 350 calories to about 400 calories, about 350 calories to about 450 calories, or about 400 calories to about 450 calories. In some embodiments, a single serving of the protein formulations comprises about 150 calories, about 200 calories, about 250 calories, about 300 calories, about 350 calories, about 400 calories, or about 450 calories. In some embodiments, a single serving of the protein formulations comprises about 215 calories. In some embodiments, a single serving of the protein formulations comprises about 300 calories.

### Applications

### Slow Digestive Release Protein Formulations

In some embodiments, the protein formulations of the present disclosure have a slow digestive release. The slow-digesting protein formulations are capable of improving or preventing problems linked with various physiological or physiopathological states. Indeed, the protein materials with a slow rate of digestion can act by optimizing the postprandial protein gain, by avoiding excessive functioning for key organs or for certain enzymes, by optimizing treatments with L-DOPA, and by increasing the sensation of satiety. The conditions governing the use of these proteins will depend in particular on the categories of people concerned.

In the context of the optimization of the postprandial protein gain, cases of undernourishment may be treated. Undernourishment frequently exists in elderly individuals or during diseases which comprise a substantial loss of body proteins—renal insufficiency, severe burns, trauma, surgical or infectious stress, inflammation, cancer or AIDS. This metabolic state manifests itself by a negative nitrogen balance which is the consequence of a fusion of the body, and more particularly muscle, proteins. Indeed, the muscle proteins are degraded so as to provide energy to the body and allow the redistribution of the amino acids to the synthesis of specific proteins. In cases of undernourishment, the ingestion of slow-digesting protein formulation is capable of limiting this protein loss, by optimizing the postprandial protein gain. These protein formulations ought to increase the rate of physiological recovery, resistance to attacks, the quality of life and therefore the vital prognosis.

Renal abnormality, in the broad sense of the term, is an example of the use of the slow-digesting protein formulation which is not solely based on the optimization of the postprandial protein gain, although it is an essential component thereof. Indeed, during renal abnormalities, patients are subjected to a strict hypoprotein diet so as to reduce the production of nitrogenous waste. It is commonly accepted that such a diet has a favorable effect on the general condition, the quality of life and even on the renal function. However, this diet is very poorly tolerated by patients. The ingestion of slow-digesting protein formulation contributes toward: (1) reducing the production of nitrogen which should be subsequently eliminated by the kidneys; (2) distributing this production over a much longer period; and (3) increasing the satiating action of this type of protein in order to ensure better tolerance of the diet. Proteins with a slow rate of digestion are consequently particularly suitable for the nutrition of patients with renal disorders.

Likewise, the slow-digesting protein formulation may be prescribed for patients with pathological hepatic conditions. After a meal composed of various nitrogenous compounds (proteins, peptides, amino acids), the liver will try to maintain the amino acid concentration within physiological limits by breaking down a portion of the amino acids derived from the diet. A moderate arrival of dietary amino acids is capable of reducing the excessive activity of an organ which exhibits pathological conditions and which will consequently make it possible to avoid excessive work. In addition, the slow-digesting protein formulation induces a better postprandial protein gain. During a deficiency in proteolytic pancreatic enzymes, the ingestion of slow-digesting protein formulation can contribute toward improving the digestion process. This benefit is brought about by the reduction in the quantity of substrate to be hydrolyzed by the proteolytic enzymes of the pancreas and therefore by the obtaining of a better enzyme/substrate ratio. Furthermore, with the slow-digesting protein material, there is a better postprandial protein gain.

The use of slow-digesting protein formulation can also be envisaged for people who are not undernourished, such as premature babies, newborns, children, obese individuals and elderly persons, for example. The ingestion of slow-digesting protein material, in premature babies, newborns or children who are not undernourished, by providing a better yield of use of the dietary proteins, is capable of promoting body growth.

The slow-digesting protein material, by reducing the food intake by a satiating mechanism, may be administered to people with disorders of weight homeostasis (obesity) or during episodes of bulimia. It can limit the reduction in the protein mass subsequent to being on a low-caloric diet. These two combined factors make it possible to reduce their fatty mass with, on the one hand, greater ease for reducing their supplies and, on the other hand, a better preservation of their protein mass. In elderly individuals, compared with young individuals, there is a reduction in the body protein mass, a reduction which has an influence on the autonomy, the resistance to attacks (diseases, various stresses) and the ability to recover from these attacks. Furthermore, aging is associated with a reduction in renal activity. The slow-digesting protein material, by therefore allowing better preservation of the protein mass, thus makes it possible to avoid renal excesses. The protein formulation with a slowed rate of digestion, by providing the amino acids in a more continuous and regular manner, makes it possible to promote the synthesis of novel tissue materials which are involved in the processes of wound healing or of regeneration of biological tissues.

### Therapeutic Ready-to-Use Therapeutic Food

In some embodiments, the protein formulations of the disclosure are therapeutic food, useful in feeding patient. In some embodiments, the protein formulations described herein are used to feed patients with chronic conditions. In some embodiments, the protein formulations described herein are used to feed patient recovering from surgery. In some embodiments, the protein formulations described herein are used to feed patients undergoing a therapy, patients who will undergo a therapy, or patients who are recovering from a therapy, for example chemotherapy. In some embodiments the protein formulations described herein are used to treat patients who suffer from a reduced appetite due to a disease, treatment or a therapy. In some embodiments the protein formulations described herein are used to treat patients who suffer from nausea. In some embodiments, the protein formulations described herein are energy-dense and/or micronutrient-enriched and as such provide superior nutrition to patients, as compared to foods made with conventional proteins.

In some embodiments, the protein formulations useful as therapeutic food are high density protein formulations, which provide high protein availability and slow release carbohydrate source. The therapeutic foods described herein provide slow-release of carbohydrates and/or proteins. In some embodiments, the protein formulations disclosed herein are fat-mediated formulation, which enables digestive release of the nutrients (e.g. protein and/or the carbohydrates).

In some examples, the protein formulations useful as therapeutic food have balanced electrolyte content and provide a low osmolality load. These properties are beneficial in preventing osmolar shock for patients suffering from dehydration, or for patients who are otherwise hydration compromised. These properties are also particularly useful for feeding patients sensitive to nausea, for example with nausea due to a medical condition and/or a therapy.

In some embodiments, the therapeutic food disclosed herein have zero or near zero sugar content and can be used for feeding insulin-sensitive patients and/or glycemic sensitive patients.

In some examples, the protein formulations useful as therapeutic food comprise protein, fat and carbohydrates. In some examples, the amount of protein in the protein formulations useful as therapeutic food is from about 20 wt % to about 75 wt %. In some examples, the amount of protein in the protein formulations useful as therapeutic food is from at least about 20 wt %. In some examples, the amount of protein in the protein formulations useful as therapeutic food is from at most about 75 wt %. In some examples, the amount of protein in the protein formulations useful as therapeutic food is from about 20 wt % to about 25 wt %, about 20 wt % to about 30 wt %, about 20 wt % to about 35 wt %, about 20 wt % to about 40 wt %, about 20 wt % to about 45 wt %, about 20 wt % to about 50 wt %, about 20 wt % to about 55 wt %, about 20 wt % to about 60 wt %, about 20 wt % to about 65 wt %, about 20 wt % to about 70 wt %, about 20 wt % to about 75 wt %, about 25 wt % to about 30 wt %, about 25 wt % to about 35 wt %, about 25 wt % to about 40 wt %, about 25 wt % to about 45 wt %, about 25 wt % to about 50 wt %, about 25 wt % to about 55 wt %, about 25 wt % to about 60 wt %, about 25 wt % to about 65 wt %, about 25 wt % to about 70 wt %, about 25 wt % to about 75 wt %, about 30 wt % to about 35 wt %, about 30 wt % to about 40 wt %, about 30 wt % to about 45 wt %, about 30 wt % to about 50 wt %, about 30 wt % to about 55 wt %, about 30 wt % to about 60 wt %, about 30 wt % to about 65 wt %, about 30 wt % to about 70 wt %, about 30 wt % to about 75 wt %, about 35 wt % to about 40 wt %, about 35 wt % to about 45 wt %, about 35 wt % to about 50 wt %, about 35 wt % to about 55 wt %, about 35 wt % to about 60 wt %, about 35 wt % to about 65 wt %, about 35 wt % to about 70 wt %, about 35 wt % to about 75 wt %, about 40 wt % to about 45 wt %, about 40 wt % to about 50 wt %, about 40 wt % to about 55 wt %, about 40 wt % to about 60 wt %, about 40 wt % to about 65 wt %, about 40 wt % to about 70 wt %, about 40 wt % to about 75 wt %, about 45 wt % to about 50 wt %, about 45 wt % to about 55 wt %, about 45 wt % to about 60 wt %, about 45 wt % to about 65 wt %, about 45 wt % to about 70 wt %, about 45 wt % to about 75 wt %, about 50 wt % to about 55 wt %, about 50 wt % to about 60 wt %, about 50 wt % to about 65 wt %, about 50 wt % to about 70 wt %, about 50 wt % to about 75 wt %, about 55 wt % to about 60 wt %, about 55 wt % to about 65 wt %, about 55 wt % to about 70 wt %, about 55 wt % to about 75 wt %, about 60 wt % to about 65 wt %, about 60 wt % to about 70 wt %, about 60 wt % to about 75 wt %, about 65 wt % to about 70 wt %, about 65 wt % to about 75 wt %, or about 70 wt % to about 75 wt %. In some examples, the amount of protein in the protein formulations useful as therapeutic food is from about 20 wt %, about 25 wt %, about 30 wt %, about 35 wt %, about 40 wt %, about 45 wt %, about 50 wt %, about 55 wt %, about 60 wt %, about 65 wt %, about 70 wt %, or about 75 wt %. In some examples, the amount of protein in the protein formulations useful as therapeutic food is from about 40 wt % to about 50 wt %. In some examples, the amount of protein in the protein formulations useful as therapeutic food is from about 40 wt % to about 45 wt %. In some examples, the amount of protein in the protein formulations useful as therapeutic food is about 42 wt %.

In some examples, the amount of fat in the protein formulations useful as therapeutic food is from about 5 wt % to about 30 wt %. In some examples, the amount of fat in the protein formulations useful as therapeutic food is from at least about 5 wt %. In some examples, the amount of fat in the protein formulations useful as therapeutic food is from at most about 30 wt %. In some examples, the amount of fat in the protein formulations useful as therapeutic food is from about 5 wt % to about 10 wt %, about 5 wt % to about 15 wt %, about 5 wt % to about 20 wt %, about 5 wt % to about 25 wt %, about 5 wt % to about 30 wt %, about 10 wt % to about 15 wt %, about 10 wt % to about 20 wt %, about 10 wt % to about 25 wt %, about 10 wt % to about 30 wt %, about 15 wt % to about 20 wt %, about 15 wt % to about 25 wt %, about 15 wt % to about 30 wt %, about 20 wt % to about 25 wt %, about 20 wt % to about 30 wt %, or about 25 wt % to about 30 wt %. In some examples, the amount of fat in the protein formulations useful as therapeutic food is from about 5 wt %, about 10 wt %, about 15 wt %, about 20 wt %, about 25 wt %, or about 30 wt %. In some examples, the amount of fat in the protein formulations useful as therapeutic food is from about 10 wt % to about 20 wt % of fat. In some examples, the amount of fat in the protein formulations useful as therapeutic food is from about 14 wt % to about 18 wt %. In some examples, the amount of fat in the protein formulations useful as therapeutic food is about 16 wt %.

In some examples, the amount of carbohydrates in the protein formulations useful as therapeutic food is from about 10 wt % to about 65 wt %. In some examples, the amount of carbohydrates in the protein formulations useful as therapeutic food is from at least about 10 wt %. In some examples, the amount of carbohydrates in the protein formulations useful as therapeutic food is from at most about 65 wt %. In some examples, the amount of carbohydrates in the protein formulations useful as therapeutic food is from about 10 wt % to about 15 wt %, about 10 wt % to about 20 wt %, about 10 wt % to about 25 wt %, about 10 wt % to about 30 wt %, about 10 wt % to about 35 wt %, about 10 wt % to about 40 wt %, about 10 wt % to about 45 wt %, about 10 wt % to about 50 wt %, about 10 wt % to about 55 wt %, about 10 wt % to about 60 wt %, about 10 wt % to about 65 wt %, about 15 wt % to about 20 wt %, about 15 wt % to about 25 wt %, about 15 wt % to about 30 wt %, about 15 wt % to about 35 wt %, about 15 wt % to about 40 wt %, about 15 wt % to about 45 wt %, about 15 wt % to about 50 wt %, about 15 wt % to about 55 wt %, about 15 wt % to about 60 wt %, about 15 wt % to about 65 wt %, about 20 wt % to about 25 wt %, about 20 wt % to about 30 wt %, about 20 wt % to about 35 wt %, about 20 wt % to about 40 wt %, about 20 wt % to about 45 wt %, about 20 wt % to about 50 wt %, about 20 wt % to about 55 wt %, about 20 wt % to about 60 wt %, about 20 wt % to about 65 wt %, about 25 wt % to about 30 wt %, about 25 wt % to about 35 wt %, about 25 wt % to about 40 wt %, about 25 wt % to about 45 wt %, about 25 wt % to about 50 wt %, about 25 wt % to about 55 wt %, about 25 wt % to about 60 wt %, about 25 wt % to about 65 wt %, about 30 wt % to about 35 wt %, about 30 wt % to about 40 wt %, about 30 wt % to about 45 wt %, about 30 wt % to about 50 wt %, about 30 wt % to about 55 wt %, about 30 wt % to about 60 wt %, about 30 wt % to about 65 wt %, about 35 wt % to about 40 wt %, about 35 wt % to about 45 wt %, about 35 wt % to about 50 wt %, about 35 wt % to about 55 wt %, about 35 wt % to about 60 wt %, about 35 wt % to about 65 wt %, about 40 wt % to about 45 wt %, about 40 wt % to about 50 wt %, about 40 wt % to about 55 wt %, about 40 wt % to about 60 wt %, about 40 wt % to about 65 wt %, about 45 wt % to about 50 wt %, about 45 wt % to about 55 wt %, about 45 wt % to about 60 wt %, about 45 wt % to about 65 wt %, about 50 wt % to about 55 wt %, about 50 wt % to about 60 wt %, about 50 wt % to about 65 wt %, about 55 wt % to about 60 wt %, about 55 wt % to about 65 wt %, or about 60 wt % to about 65 wt %. In some examples, the amount of carbohydrates in the protein formulations useful as therapeutic food is from about 10 wt %, about 15 wt %, about 20 wt %, about 25 wt %, about 30 wt %, about 35 wt %, about 40 wt %, about 45 wt %, about 50 wt %, about 55 wt %, about 60 wt %, or about 65 wt %. In some examples, the amount of carbohydrates in the protein formulations useful as therapeutic food is from about 25 wt % to about 35 wt %, for example about 27 wt % to about 32 wt %. In some examples, the amount of carbohydrates in the protein formulations useful as therapeutic food is about 30 wt %.

The protein formulations useful as therapeutic food may further comprise one or more additional components disclosed herein. For example, the protein formulations useful as therapeutic food further comprise one more vitamins, minerals, electrolytes, flavoring agents, stabilizing agents, sweeteners, and emulsifiers disclosed herein. In some embodiments, the protein formulations useful as therapeutic food are sweetened using plant-based non-nutritive sweetener. In some embodiments, the protein formulations useful as therapeutic food are flavored to suit needs of the target patient population. In some embodiments, the protein formulations useful as therapeutic food comprise a stabilizing agent. Any suitable stabilizing agent can be used, including those described in this disclosure. In some embodiments, the protein formulations useful as therapeutic food are stabilized with sterins, tocopherols and/or ascorbyl palmitate.

In some embodiments, the protein formulations useful as therapeutic food are supplemented with cannabis extract, for example to feed patients needing anti-emetic or pain management therapies.

In some embodiments, the total amount of the vitamins, minerals, electrolytes, flavoring agents, stabilizing agents, sweeteners, and/or emulsifiers in the protein formulations useful as therapeutic food is about 5% to about 25%, for example about 5%-about 20%, about 5% to about 15%, about 5% to about 10%, about 10% to about 25%, about 10% to about 20%, about 10% to about 15%, about 15% to about 25%, about 15% to about 20%, about 200% to about 25%. In some embodiments, the total amount of the vitamins, minerals, electrolytes, flavoring agents, stabilizing agents, sweeteners, and/or emulsifiers in the protein formulations useful as therapeutic food is about 10% to about 15%, for example about 12%.

### Ketogenic Diets and Fast-Mimicking Diets

In some embodiments, the protein formulations of the invention are useful as ketogenic and/or fast mimicking diet formula. Fast-mimicking diets and ketogenic diets have emerged as very efficient strategies for weight loss and diabetes prevention and management. These diets are very low in carbohydrates and sugar and high in fat and proteins. Research indicates that it is very difficult to adhere to such diets because of the unavailability of foods with the adequate composition and potentially cumbersome preparation. The protein formulations of the disclosure provide composition for such diets.

In some examples, the protein formulations useful as ketogenic and/or fast mimicking diet comprise protein, fat and carbohydrates. In some examples, the amount of protein in the protein formulations useful as ketogenic and/or fast mimicking diet is from about 20 wt % to about 75 wt %. In some examples, the amount of protein in the protein formulations useful as ketogenic and/or fast mimicking diet is from at least about 20 wt %. In some examples, the amount of protein in the protein formulations useful as ketogenic and/or fast mimicking diet is from at most about 75 wt %. In some examples, the amount of protein in the protein formulations useful as ketogenic and/or fast mimicking diet is from about 20 wt % to about 25 wt %, about 20 wt % to about 30 wt %, about 20 wt % to about 35 wt %, about 20 wt % to about 40 wt %, about 20 wt % to about 45 wt %, about 20 wt % to about 50 wt %, about 20 wt % to about 55 wt %, about 20 wt % to about 60 wt %, about 20 wt % to about 65 wt %, about 20 wt % to about 70 wt %, about 20 wt % to about 75 wt %, about 25 wt % to about 30 wt %, about 25 wt % to about 35 wt %, about 25 wt % to about 40 wt %, about 25 wt % to about 45 wt %, about 25 wt % to about 50 wt %, about 25 wt % to about 55 wt %, about 25 wt % to about 60 wt %, about 25 wt % to about 65 wt %, about 25 wt % to about 70 wt %, about 25 wt % to about 75 wt %, about 30 wt % to about 35 wt %, about 30 wt % to about 40 wt %, about 30 wt % to about 45 wt %, about 30 wt % to about 50 wt %, about 30 wt % to about 55 wt %, about 30 wt % to about 60 wt %, about 30 wt % to about 65 wt %, about 30 wt % to about 70 wt %, about 30 wt % to about 75 wt %, about 35 wt % to about 40 wt %, about 35 wt % to about 45 wt %, about 35 wt % to about 50 wt %, about 35 wt % to about 55 wt %, about 35 wt % to about 60 wt %, about 35 wt % to about 65 wt %, about 35 wt % to about 70 wt %, about 35 wt % to about 75 wt %, about 40 wt % to about 45 wt %, about 40 wt % to about 50 wt %, about 40 wt % to about 55 wt %, about 40 wt % to about 60 wt %, about 40 wt % to about 65 wt %, about 40 wt % to about 70 wt %, about 40 wt % to about 75 wt %, about 45 wt % to about 50 wt %, about 45 wt % to about 55 wt %, about 45 wt % to about 60 wt %, about 45 wt % to about 65 wt %, about 45 wt % to about 70 wt %, about 45 wt % to about 75 wt %, about 50 wt % to about 55 wt %, about 50 wt % to about 60 wt %, about 50 wt % to about 65 wt %, about 50 wt % to about 70 wt %, about 50 wt % to about 75 wt %, about 55 wt % to about 60 wt %, about 55 wt % to about 65 wt %, about 55 wt % to about 70 wt %, about 55 wt % to about 75 wt %, about 60 wt % to about 65 wt %, about 60 wt % to about 70 wt %, about 60 wt % to about 75 wt %, about 65 wt % to about 70 wt %, about 65 wt % to about 75 wt %, or about 70 wt % to about 75 wt %. In some examples, the amount of protein in the protein formulations useful as ketogenic and/or fast mimicking diet is from about 20 wt %, about 25 wt %, about 30 wt %, about 35 wt %, about 40 wt %, about 45 wt %, about 50 wt %, about 55 wt %, about 60 wt %, about 65 wt %, about 70 wt %, or about 75 wt %. In some examples, the amount of protein in the protein formulations useful as ketogenic and/or fast mimicking diet is from about 20 wt % to about 40 wt %. In some examples, the amount of protein in the protein formulations useful as ketogenic and/or fast mimicking diet is from about 25 wt % to about 35 wt %. In some examples, the amount of protein in the protein formulations useful as ketogenic and/or fast mimicking diet is about 30 wt %.

In some examples, the amount of fat in the protein formulations useful as ketogenic and/or fast mimicking diets is from about 20 wt % to about 40 wt %. In some examples, the amount of fat in the protein formulations useful as ketogenic and/or fast mimicking diets is from at least about 20 wt %. In some examples, the amount of fat in the protein formulations useful as ketogenic and/or fast mimicking diets is from at most about 40 wt %. In some examples, the amount of fat in the protein formulations useful as ketogenic and/or fast mimicking diets is from about 20 wt % to about 25 wt %, about 20 wt % to about 30 wt %, about 20 wt % to about 35 wt %, about 20 wt % to about 40 wt %, about 25 wt % to about 30 wt %, about 25 wt % to about 35 wt %, about 25 wt % to about 40 wt %, about 30 wt % to about 35 wt %, about 30 wt % to about 40 wt %, or about 35 wt % to about 40 wt %. In some examples, the amount of fat in the protein formulations useful as ketogenic and/or fast mimicking diets is from about 20 wt %, about 25 wt %, about 30 wt %, about 35 wt %, or about 40 wt %. In some examples, the amount of fat in the protein formulations useful as ketogenic and/or fast mimicking diets is from about 25 wt % to about 35 wt %. In some examples, the amount of fat in the protein formulations useful as ketogenic and/or fast mimicking diets is about 30 wt %.

In some examples, the amount of carbohydrates in the protein formulations useful as ketogenic and/or fast mimicking diets is from about 10 wt % to about 65 wt %. In some examples, the amount of carbohydrates in the protein formulations useful as ketogenic and/or fast mimicking diets is from at least about 10 wt %. In some examples, the amount of carbohydrates in the protein formulations useful as ketogenic and/or fast mimicking diets is from at most about 65 wt %. In some examples, the amount of carbohydrates in the protein formulations useful as ketogenic and/or fast mimicking diets is from about 10 wt % to about 15 wt %, about 10 wt % to about 20 wt %, about 10 wt % to about 25 wt %, about 10 wt % to about 30 wt %, about 10 wt % to about 35 wt %, about 10 wt % to about 40 wt %, about 10 wt % to about 45 wt %, about 10 wt % to about 50 wt %, about 10 wt % to about 55 wt %, about 10 wt % to about 60 wt %, about 10 wt % to about 65 wt %, about 15 wt % to about 20 wt %, about 15 wt % to about 25 wt %, about 15 wt % to about 30 wt %, about 15 wt % to about 35 wt %, about 15 wt % to about 40 wt %, about 15 wt % to about 45 wt %, about 15 wt % to about 50 wt %, about 15 wt % to about 55 wt %, about 15 wt % to about 60 wt %, about 15 wt % to about 65 wt %, about 20 wt % to about 25 wt %, about 20 wt % to about 30 wt %, about 20 wt % to about 35 wt %, about 20 wt % to about 40 wt %, about 20 wt % to about 45 wt %, about 20 wt % to about 50 wt %, about 20 wt % to about 55 wt %, about 20 wt % to about 60 wt %, about 20 wt % to about 65 wt %, about 25 wt % to about 30 wt %, about 25 wt % to about 35 wt %, about 25 wt % to about 40 wt %, about 25 wt % to about 45 wt %, about 25 wt % to about 50 wt %, about 25 wt % to about 55 wt %, about 25 wt % to about 60 wt %, about 25 wt % to about 65 wt %, about 30 wt % to about 35 wt %, about 30 wt % to about 40 wt %, about 30 wt % to about 45 wt %, about 30 wt % to about 50 wt %, about 30 wt % to about 55 wt %, about 30 wt % to about 60 wt %, about 30 wt % to about 65 wt %, about 35 wt % to about 40 wt %, about 35 wt % to about 45 wt %, about 35 wt % to about 50 wt %, about 35 wt % to about 55 wt %, about 35 wt % to about 60 wt %, about 35 wt % to about 65 wt %, about 40 wt % to about 45 wt %, about 40 wt % to about 50 wt %, about 40 wt % to about 55 wt %, about 40 wt % to about 60 wt %, about 40 wt % to about 65 wt %, about 45 wt % to about 50 wt %, about 45 wt % to about 55 wt %, about 45 wt % to about 60 wt %, about 45 wt % to about 65 wt %, about 50 wt % to about 55 wt %, about 50 wt % to about 60 wt %, about 50 wt % to about 65 wt %, about 55 wt % to about 60 wt %, about 55 wt % to about 65 wt %, or about 60 wt % to about 65 wt %. In some examples, the amount of carbohydrates in the protein formulations useful as ketogenic and/or fast mimicking diets is from about 10 wt %, about 15 wt %, about 20 wt %, about 25 wt %, about 30 wt %, about 35 wt %, about 40 wt %, about 45 wt %, about 50 wt %, about 55 wt %, about 60 wt %, or about 65 wt %. In some examples, the amount of carbohydrates in the protein formulations useful as ketogenic and/or fast mimicking diets is from about 25 wt % to about 35 wt %, for example about 27 wt % to about 32 wt %. In some examples, the amount of carbohydrates in the protein formulations useful as ketogenic and/or fast mimicking diets is about 30 wt %.

The protein formulations useful as ketogenic and/or fast mimicking diets may further comprise one or more additional components disclosed herein. For example, the protein formulations useful as ketogenic and/or fast mimicking diets further comprise one more vitamins, minerals, electrolytes, flavoring agents, stabilizing agents, sweeteners, and emulsifiers disclosed herein. In some embodiments, the protein formulations useful as ketogenic and/or fast mimicking diets are sweetened using plant-based non-nutritive sweetener. In some embodiments, the protein formulations useful as ketogenic and/or fast mimicking diets are flavored to suit needs of the target patient population. In some embodiments, the protein formulations useful as ketogenic and/or fast mimicking diets comprise a stabilizing agent. Any suitable stabilizing agent can be used, including those described in this disclosure. In some embodiments, the protein formulations useful as ketogenic and/or fast mimicking diets are stabilized with sterins, tocopherols and/or ascorbyl palmitate.

In some embodiments, the protein formulations useful as ketogenic and/or fast mimicking diets are supplemented with cannabis extract, for example to feed patients needing anti-emetic or pain management therapies.

In some embodiments, the total amount of the vitamins, minerals, electrolytes, flavoring agents, stabilizing agents, sweeteners, and/or emulsifiers in the protein formulations useful as therapeutic food is about 5% to about 25%, for example about 5%-about 20%, about 5% to about 15%, about 5% to about 10%, about 10% to about 25%, about 10% to about 20%, about 10% to about 15%, about 15% to about 25%, about 15% to about 20%, about 200% to about 25%. In some embodiments, the total amount of the vitamins, minerals, electrolytes, flavoring agents, stabilizing agents, sweeteners, and/or emulsifiers in the protein formulations useful as ketogenic and/or fast mimicking diets is about 8% to about 12%, for example about 10%.

In some embodiments, the protein formulation useful as ketogenic and/or fast mimicking diets are used in feeding patients suffering from epilepsy, obesity, diabetes, high cholesterol, cancers, inflammatory diseases, trauma, behavioral disorders, senile dementia, autism, cerebral spinal cord injury, migraine, neurodegenerative and degenerative diseases. In some embodiments, the ketogenic and/or fast mimicking diets are useful in treating or controlling epilepsy, obesity, diabetes, high cholesterol, cancers, inflammatory diseases, trauma, behavioral disorders, senile dementia, migraine, neurodegenerative and degenerative diseases. In some embodiments, the protein formulation useful as ketogenic and/or fast mimicking diets is used in feeding patients suffering from obesity. In some embodiments, the ketogenic and/or fast mimicking diets are useful in treating or controlling obesity. In some embodiments, the ketogenic and/or fast mimicking diets are useful in feeding patients in need of, or wanting weight, loos.

### Ready-to-Use Therapeutic Foods (RUTFs) and Ready-to-Use Supplemental Food (RF)

In some embodiments, the protein formulations provided herein are useful as Ready-to-use-therapeutic food (RUTF). In various embodiments, the RUTFs provided herein differ from conventional RUTF, in that they do not comprise any milk protein. For example the RUTFs provided herein do not comprise any non-fat dried milk (NFDM), whey protein concentrate (WPC) or milk protein concentrate (MPC), which tend to be increasingly expensive. In these embodiments, the RUTFs comprise an alternative source of animal-based protein. In some examples this alternate protein is protein from an aquatic animal, for example fish protein. These RUTFs comprise no, or essentially no lactose. In various embodiments these RUTFs maintain an ideal amino acid profile (PDCAAS score). In various embodiments, the RUTFs provided herein have a nutritional profile similar to a traditional F-75 or F-100 milk-based diet.

In some embodiments, the protein formulations provided herein are useful as Ready-to-use-supplemental food (RUSF). In various embodiments, the RUSFs provided herein differ from the conventional RUSFs in that they do not comprise any dairy protein. As such the RUSFs provided herein comprise no or essentially no lactose. In some embodiments, the RUSFs disclosed herein provide no plant-based protein. For example, the RUSF of the disclosure comprise no pea or soy protein. In some embodiments, the RUSFs disclosed herein provide no plant-based protein and no milk based protein. In these examples, the RUSFs described herein comprise an alternate protein instead of the milk and/or plant based-protein. In some examples, the alternate protein is protein from an aquatic animal, for example fish protein.

In various examples, the RUSFs and RUTFs of the disclosure provide improved palatability and/or good dispersibility as compared to the traditional RUSFs and RUTFs. In some embodiments, the RUSFs and RUTFs described herein comprise protein powder that is sourced from “green” zero carbon footprint and low water consumption sourcing and are therefore preferred compared to the traditional RUSFs and RUTFs.

Typically, the RUTFs and RUSFs disclosed herein will follow similar required nutrient formulations as mandated by the GMOs. However in various embodiments, the RUTFs and the RUSFs disclosed herein are formulated from protein powder with high protein content (e.g. >90%, >91%, >92%, >93%, >94%, >95%, >96%, >97%, >98%, or >99% protein) and low carbohydrate content (e.g. <10%, <9%, <8%, <7%, <5%, <4%, <3%, <2%, <1%, <0.5%, <0.25%, or <0.1%). This high quality of the protein powder provides some added flexibility in formulation of these RUSFs and RUTFs, when compared to use of NFDM, MPC, WPC, soy protein isolate (SPI) and pea protein concentrate.

### EXAMPLES

The following specific examples are illustrative and non-limiting. The examples described herein reference and provide non-limiting support to the various embodiments described in the preceding sections.

### Example 1. Isolation of Low Molecular Weight Protein

Two different kinds of fish samples were provided; a fine grind sample of fish from the west coast of the United States and a coarse grind sample of fish from the east coast of the United States. A slurry was prepared by mixing 93 grams of raw fish with 186 milliliters of isopropanol alcohol (“IPA”). The mixture was then heated to 70 degrees Celsius and stirred for two hours prior to filtering. Vacuum filtration was performed on the slurry using the Nutsche filter for 75 seconds. The filter area was 56 cm². The filter media was polypropylene double layer weaves with mesh sizes ranging from 30 μm to 7 μm. The filter media with 30 μm sized mesh allowed some visible solids to pass, while the 7 μm filter media was successful in generating a clear filtrate. Both reslurry and displacement extraction methods were used to extract additional oils after the first extraction. For reslurry extractions, the filter cake derived from the first filtering step was mixed 186 milliliters of IPA. The temperature was maintained at 70 degrees Celsius and stirred for two hours. For the displacement extraction method, 186 milliliters of IPA at 70 degrees Celsius was poured on top of the filter cake and allowed to pass through under vacuum for 75 seconds. The filter cake resulting from both processes was allowed to dry in a vacuum oven for 60 seconds. The resulting filter cake was 14 mm in height. The filter cake was discharged by removing the bulk of the cake and scraping the remaining solids from the media and walls of the filter. The filter media was rinsed on both sides using high pressure tap water from a sink faucet.

This method can be scaled up using a conveyance system and belt filters of particular sizes to support throughput of 1250 kg/hour dry solids.

### Example 2. Properties of Protein (Molecular Weight)

The molecular weight distribution and bioactive peptide profile of the protein compositions described herein (“APP”) was analyzed and compared to Fish Protein Powder manufactured by Aroma, New Zealand (“FPP”), Pea Protein Isolate 80% distributed by Bright Pharma Ingredients, USA (“PPI”), and Whey Protein Isolate 895 distributed by Batory Foods, Inc. (“WPI”) using conventional analysis techniques. It was discovered that the protein compositions described herein comprise a molecular weight distribution profile with a significant percentage (ca. 69%) of peptides of 5,900 Da or less. In contrast to other proteins (even hydrolyzed) having a percentage of low molecular weight, the composition of bioactive peptides in the range of 4 to 27%. Table 1 shows representative results.

TABLE 1
Molecular Weight Distribution and Peptides
Profile of Various Proteins

| Type of | % of Peptides | Molecular Weight |
|---|---|---|
| Proteins | fraction | Distribution (KDa) |
| APP | 68.97 | 5.90 |
| FPP | 26.98 | 5.10 |
| PPI | 3.19 | 5.00 |
| WPI | 50.428 | 19.40 |

APP: Advance Protein Powder/A Perfect Protein ® (APP) manufactured by Advance International, USA.
FPP: Fish Protein Powder manufactured by Aroma, New Zealand.
PPI: Pea Protein Isolate 80% distributed by Bright Pharma Ingredients, USA
WPI: Whey Protein Isolate 895 distributed by Batory Foods, Inc.

### Example 3. Properties of Protein (Chemical Structure)

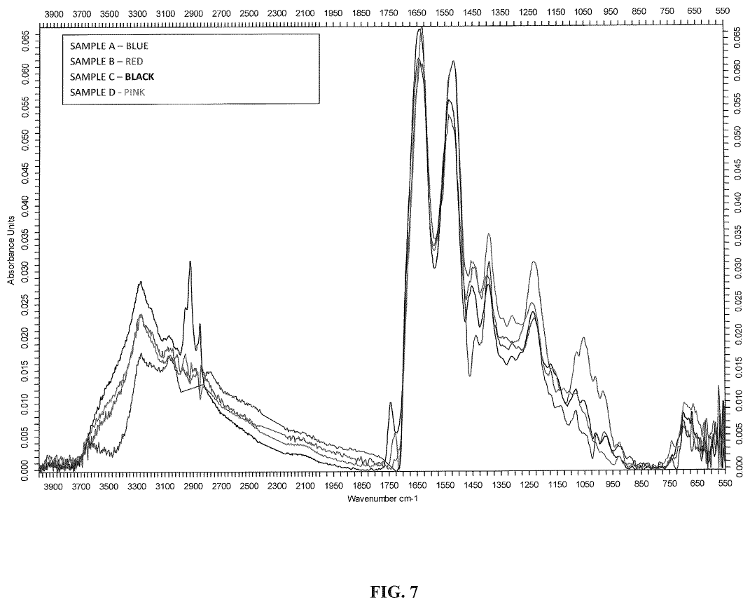

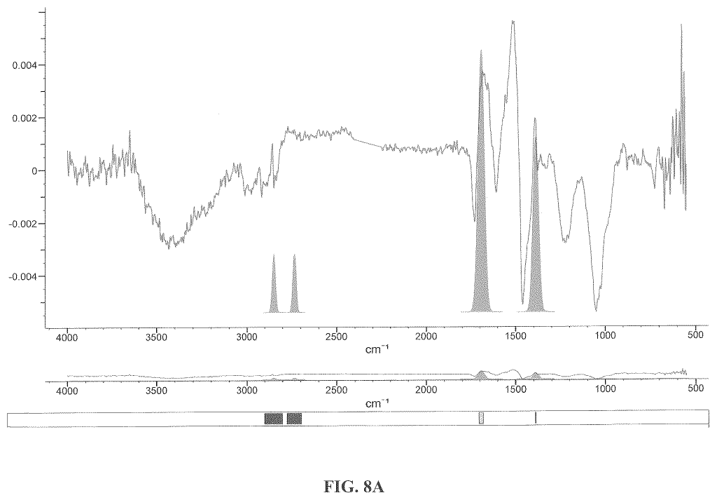

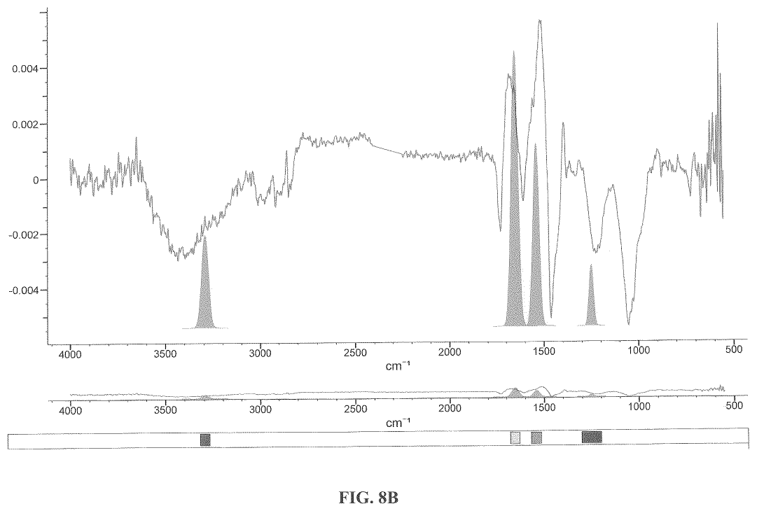

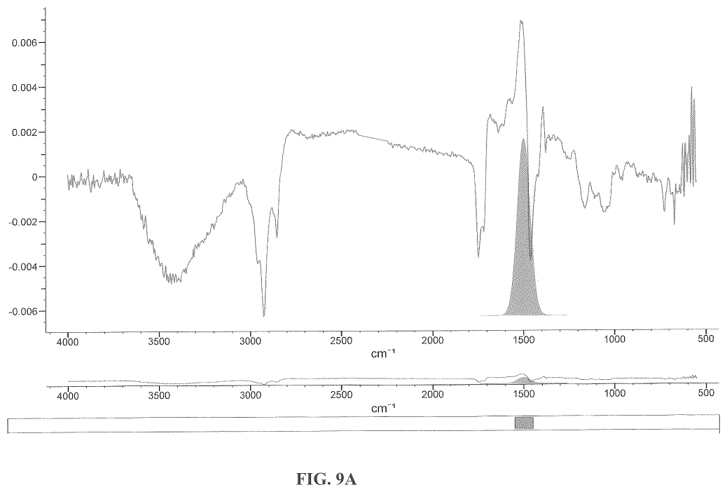

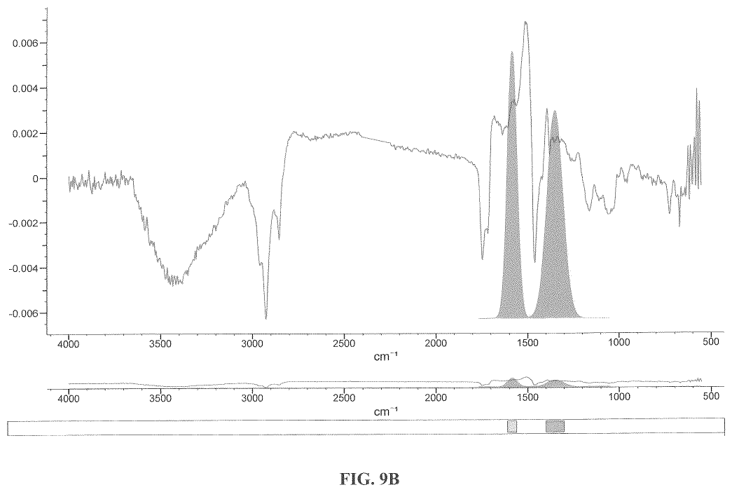

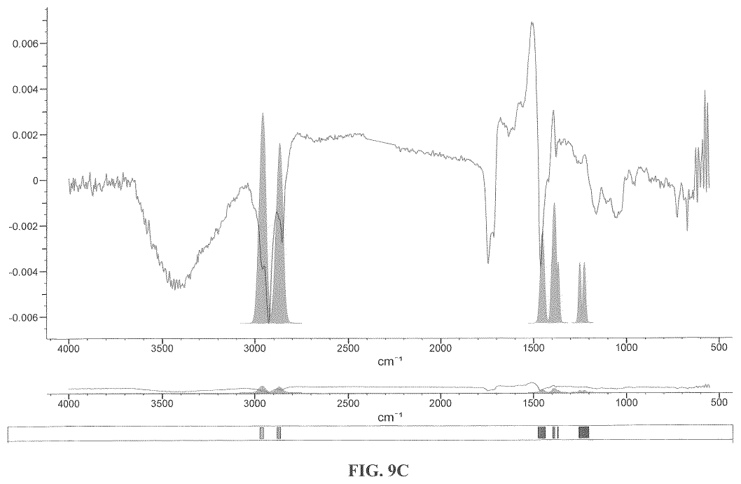

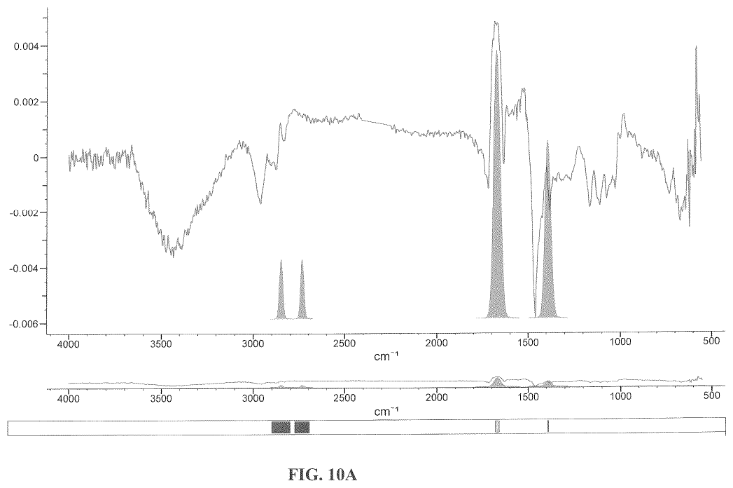

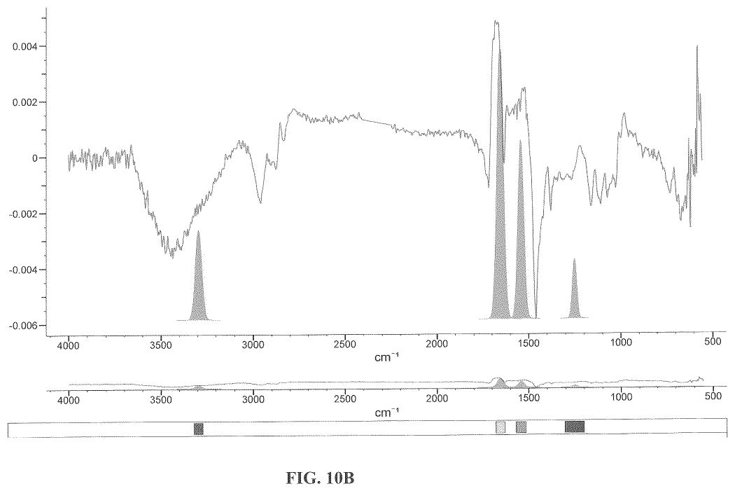

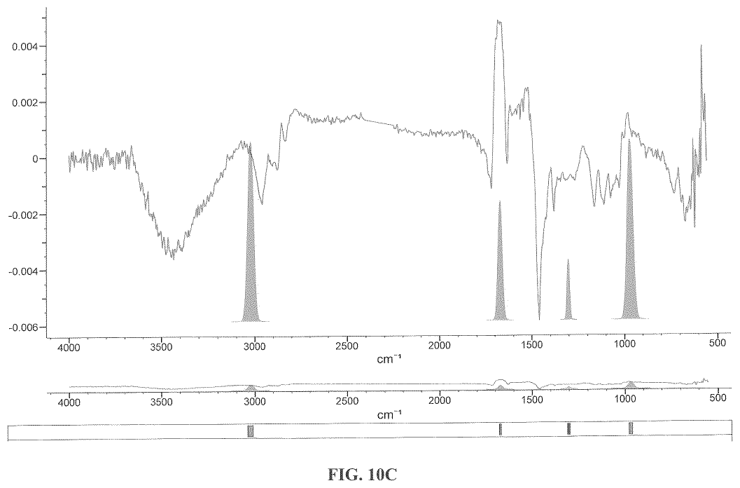

Fourier transform infrared (FTIR) spectroscopy technique was used to compare APP to commercially available proteins (FPP, PPI, and WPI). Spectral subtraction (APP-FPP, APP-PPI, APP-WPI) was used to help identify peaks that are unique APP. This was done via an algorithm that shows peaks present in APP that are not present in other proteins. The colored peaks are areas where the wavelength of the spectroscopy peaks are located. A significantly different chemical structure than conventional protein was detected in APP. The presence of features involving aldehydes, amides, triazenes, carboxylic acids, and alkanes was revealed in APP. FIG. 7 shows the total spectra absorptions of APP, FPP, PPI, and WPI. FIGS. 8A-8B shows APP's spectral absorption subtracted from FPP. FIG. 8A shows the presence of aldehyde groups in APP while aldehyde groups are absent in FPP. FIG. 8B shows the presence of amide groups in APP while amide groups are absent in FPP. FIGS. 9A-9C shows APP's spectral absorption subtracted from PPI. FIG. 9A shows the presence of triazene groups in APP while triazene groups are absent in PPI. FIG. 9B shows the presence of carboxylic acid groups in APP while carboxylic groups are absent in PPI. FIG. 9C shows the presence of alkane groups in APP while alkane groups are absent in PPI. FIGS. 10A-10C shows APP's spectral absorption subtracted from WPI. FIG. 10A shows the presence of aldehyde groups in APP while aldehyde groups are absent in WPI. FIG. 10B shows the presence of amide groups in APP while amide groups are absent in WPI. FIG. 10C shows the presence of alkene groups in APP while alkene groups are absent in WPI. The infrared (“IR”) peaks used for identifying the above structures are summarized in Table 2 below.

TABLE 2
IR peak ranges for Aldehydes, Triazenes,
Carboxylic Acids, Alkenes, and Alkanes

| Classification | Group | IR peaks, cm⁻¹ |
|---|---|---|
| Aldehydes | CH═CH—CHO | CH 2900-2800 (stretching) |
| | | CH 2775-2695 (stretching) |
| | | C═O 1705-1680 (stretching) |
| | | CH 1392-1388 (rocking) |
| Triazenes | C—N═N—NH—C | N═N 1550-1450 (stretching) |
| Carboxylic- | RC(═O)O | C═O 1610-1560 (stretching) |
| Acids | | C═O 1400-1300 (stretching) |
| Alkenes | RCH═CHR trans | CH 3040-3010 (stretching) |
| | | C═C 1678-1668 (stretching) |
| | | CH 1310-1295 (stretching) |
| | | CH 980-960 (deformation) |
| Amides | R—C(═O)—NH—C | NH 3320-3270 (stretching) |
| | | C═O 1680-1630 (stretching) |
| | | NH 1570- 1515 (combination) |
| | | NH 1305-1200 (combination) |
| Alkanes | C—(CH₃)₃ | CH 2972-2952 (antisymmetric |
| | | stretching) |
| | | CH 2882-2862 (symmetric |
| | | stretching) |
| | | CH 1475-1435 (antisymmetric |
| | | deformation) |

### Example 4. Properties of Protein (Fish Allergen Reduction)

In a single batch of APP production, Pacific Whiting (Hake) was used as raw material for the manufacture of the APP. The parvalbumin protein (primary fish allergen) concentration of the APP was analyzed and compared to the parvalbumin protein concentration of the raw Pacific Whiting. The test results, summarized in Table 3 below, showed that parvalbumin was reduced by 99.8% in the APP, compared to parvalbumin content in raw Pacific Whiting.

TABLE 3
Parvalbumin protein concentration reduction in APP

| Sample type | (ppm) | Reduction (%) |
|---|---|---|
| Raw Pacific Whiting | 47,250 | 99.8 |
| APP | 93.7 | |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

### Example 5. Composition of an Exemplary Ready to Use Therapeutic Food

Serving size: 50 g serving size;
Packaging: Paste in a squeezable pouch
Calorie content: 215 calories
   Protein: 21 g of fish protein powder (Example 8)
   Fat: 8 g total fat including omega-3 rich fish oil
   Carbohydrates: 15 g complex carbohydrates, including fiber, from Barley
   Vitamins, electrolytes, minerals, flavorings and stabilizing agents: 6 g

### Example 6. Exemplary Ketogenic Diets and Fast-Mimicking Formula

Serving size: 50 g
Packaging: Paste in a squeezable pouch
Calorie content: 300 calories
   Protein: 15 g of fish protein powder (Example 8)
   Fat: 15 g total fat including omega-3 rich fish oil
   Carbohydrates: 15 g low GI carbohydrate source
   Vitamins, electrolytes, minerals, flavorings and stabilizing agents: 10 g

### Example 7. Exemplary Method of Making the Formulation

A carrier oil (for example vegetable oil comprising canola oil, safflower oil, soya oil, or any combination thereof) is heated to 70° C. and stabilizers and emulsifiers (for example, mono glycerides, palm oil stearin, ascorbyl palmitate, or any combination thereof) are blended to melt. To this mixture vitamins and/or minerals are added and blend well. Fish Protein Powder (Example 4) is added and the mixture is mixed until fully blended. Subsequently flavorings and sweeteners are added and mixed well. Add electrolytes and mix. Next add bulk carbohydrate components and mix until all lumps are smooth and blend is mostly homogenous.

The entire mixture is passed through a sheer pump or polishing mill (Stephan Microcut, Urschil Comitrol, or equivalent) to achieve uniform particle size of 200 μm or finer (e.g. 100-150 μm). Next, any texture-providing ingredients (toasted oats, nut pieces) or other granular components are added following the milling step. The resulting mixture is tempered to 110-130° F. to facilitate filling and handling. The product is filled into single-serve pouches or multi-serve tubs. Ideally allow to cool to <100° F. prior to packing into shipping cases to help prevent oil separation.

### Example 8. Proteins Used in the Formulations Described Herein and Exemplified in Examples 5-7

The proteins used in the formulations described in Examples 1-3 are processed by the methods and systems described herein. Table 4, as shown below, describes the composition an amino gram of solid, protein powder recovered from fish according to an embodiment of the present invention. Specifically, the yield of protein is 85.4%, moisture is 7.68%, and crude fat is 1.42%. The complexity of the amino acid structure as depicted below causes the osmolality rate, solubility rate, and PDCAAS as described herein.

TABLE 4
CERTIFICATE OF ANALYSIS
Sample Identification
Sample #: 05-5432 Advance Protein Powder, Serving = 35 g
Method:
B0202: Amino Acid Profile (Total) by AOAC 98170
PB100 NLEA Abbreviated Nutrient Package (Proximate)
Results: OF AMINO GRAM Sample #05-5432

| | | | | Theoretical |
|---|---|---|---|---|
| Test | /100 g | Serving | Units | Level |
| Protein - Food | 85.4 | 29.9 | grams | 85-90% |
| Protein = Nitrogen × 6.38 | | | | |
| Ash | 9.20 | 3.22 | grams | |
| Moisture By Vacuum Oven | 7.68 | 2.69 | grams | |
| Crude Fat By Acid Hydrolysis | 1.42 | 0.497 | grams | 0.5% |
| Calories, Calculated | 340 | 119 | calories | |

Total Amino Acid Profile

| | | | |
|---|---|---|---|
| Tryptophan | 1.06 | 0.371 | grams |
| Cysteine | 0.83 | 0.291 | grams |
| Methionine | 2.51 | 0.879 | grams |
| Aspartic Acid | 4.58 | 1.6 | grams |
| Threonine | 2.15 | 0.753 | grams |
| Serine | 1.64 | 0.574 | grams |
| Glutamic Acid | 6.64 | 2.32 | grams |
| Proline | 1.89 | 0.662 | grams |
| Glycine | 2.54 | 0.889 | grams |
| Alanine | 2.9 | 1.015 | grams |
| Valine | 2.31 | 0.809 | grams |
| Isoleucine | 2.03 | 0.711 | grams |
| Leucine | 3.51 | 1.23 | grams |
| Tyrosine | 1.54 | 0.539 | grams |
| Phenylalanine | 1.86 | 0.651 | grams |
| Lysine, Total | 3.92 | 1.37 | grams |
| Histidine | 1.22 | 0.427 | grams |
| Arginine | 2.97 | 1.04 | grams |

As shown in Table 5, specific tests conducted on the recovered solid, protein powder derived from fish. As shown, the protein has over 98% digestible protein according to the well-known Pepsin test (0.2% Pepsin). Pepsin is a material that is used to digest protein structures. The Pepsin test is used to determine how much protein is within a mixture. The test involves analyzing the amount of protein that was digested, then back calculating that amount to the original quantity of protein material in the sample undergoing analysis. The trans-fatty acid isomers are less than. 1 wt. %, and preferably less than 0.05 wt. %. The amount of cholesterol is less than. 1 wt. %, preferably less than 0.05 wt. %, and more preferably less than 0.02 wt. % of a 100 g serving.

TABLE 5
CERTIFICATE OF ANALYSIS
Sample Identification
Sample #: 05-5432 Advance Protein Powder, Serving = 35 g
Method:
B0003: Customized Analyses (Pepsin (0.2%) Digestible Protein)
B7033: Cholesterol by Gas Chromatography (GO, AOAC 994.10
O0201: Total Trans Fatty Acid by Gas Chromatography (GO. AOAC
996.06
Results: Sample #05-5432

| Test | /100 g | Serving | Units |
|---|---|---|---|
| Pepsin (0.2%) Digestible Protein | 98.1 | 34.3 | grams |
| Total Trans Fatty Acid Isomers | 0.02 | 0.007 | grams |
| Cholesterol | 0.0173 | 0.00605 | grams |

As shown in Table 6 below, an elemental scan of the solid protein power indicates the following elements present in mg per serving. Also shown below in Table 6 is the amount of each element in parts per million.

TABLE 6
CERTIFICATE OF ANALYSIS AMINOGRAM
Sample Identification:
Sample #: 05-5432 Advance Protein Powder. Serving = 35 g
Method:
AL194: Elemental Scan (65) by ICP MS
Results: Sample #05-5432

| Test | | |
|---|---|---|
| Elemental | Result (meg/serving) | Result (ppm) |
| Lithium | <35 | <1 |
| Boron | <35 | <1 |
| Magnesium | 56,000 | 1,600 |
| Phosphorus | 220,000 | 6,400 |
| Calcium | 770,000 | 22,000 |
| Titanium | 77 | 2.2 |
| Chromium | 91 | 2.6 |
| Iron | 4,600 | 130 |
| Nickle | <35 | <1 |
| Zinc | 2,070 | 59 |
| Germanium | <35 | <1 |
| Selenium | 91 | 2.6 |
| Strontium | 3,900 | 110 |
| Zirconium | <35 | <1 |
| Molybdenum | <35 | <1 |
| Rhodium | <35 | <1 |
| Silver | <35 | <1 |
| Indium | NA | NA |
| Antimony | <35 | <1 |
| Cesium | <35 | <1 |
| Lanthanum | <35 | <1 |
| Praseodymium | <35 | <1 |
| Beryllium | <35 | <1 |
| Sodium | 70,000 | 2,000 |
| Aluminum | 2,000 | 56 |
| Potassium | 190,000 | 5,500 |
| Scandium | <35 | <1 |
| Vanadium | <35 | <1 |
| Manganese | 120 | 3.3 |
| Cobalt | <35 | <1 |
| Copper | 160 | 4.7 |
| Gallium | <35 | <1 |
| Arsenic | <35 | <1 |
| Rubidium | 49 | 1.4 |
| Yttrium | <35 | <1 |
| Niobium | <35 | <1 |
| Ruthenium | <35 | <1 |
| Palladium | <35 | <1 |
| Cadmium | <35 | <1 |
| Tin | <180 | <5 |
| Tellurium | <35 | <1 |
| Barium | 63 | 1.8 |
| Cerium | <35 | <1 |
| Neodymium | <35 | <1 |
| Samarium | <35 | <1 |
| Gadolinium | <35 | <1 |
| Dysprosium | <35 | <1 |
| Erbium | <35 | <1 |

Table 7 shown below compares the nutritional content for 25 mg protein of one example of the recovered solid protein of the inventive process and system which subsequently has been milled into a powder “APP” versus 25 mg protein of commercial products on the market. APP is derived from fish. Specifically, APP has fewer calories than each of the commercial products except for NB soy. APP has fewer carbohydrates and fat than NB soy. Compared with JF soy, APP has fewer calories and less fat. Compared with each DFH whey, JF whey, GNC whey, Whey isolate and Whey concentrate, APP has fewer calories, carbohydrates, fat, saturated fat and cholesterol.

TABLE 7
Standardized to 25 grams of protein per serving

| | | DFH | JF | GNC | Whey | Whey | JF | NB |
|---|---|---|---|---|---|---|---|---|
| | APP | whey | whey′ | whey | isolate | concentrate | SOY | SOY |
| Calories | 100 | 135 | 131 | 135 | 113 | 125 | 110 | 91 |
| Protein | 25 g | 25 g | 25 g | 25 g | 25 g | 25 g | 25 g | 25 g |
| Carbohydrate | 0 g | 3 g | 3 g | 4.2 g | 2.8 g | 3.1 g | 0 g | 0.7 g |
| Fat | 0 g | 2.1 g | 1.4 g | 2.1 g | 0.7 g | 1.6 g | 0.9 g | 0.2 g |
| Saturated Fat | 0 g | 12.1 g | 2.3 g | 1.0 g | 0.5 g | 1.0 g | 0 g | 0 g |
| Cholesterol | 0 g | 31.3 mg | 69.4 mg | 72.9 mg | 2.8 mg | 64.6 mg | 0 g | 0 g |

Table 8 shown below compares chemical elements existing in 25 mg of one example of the recovered solid protein of the inventive process and system which subsequently has been milled into powder “APP” versus 25 mg protein of commercial products on the market. APP is derived from fish. Notably, the calcium, iron and zinc contents of 25 mg APP is significantly greater than for each of DFH whey, JF whey, GNC whey, Whey Isolate, Whey concentrate, JF soy and NB soy. The amount of iron present in APP is significantly greater than in each of DFH whey, JF whey, GNC whey, Whey isolate, and Whey concentrate.

TABLE 8
Comparing mineral content per 25 grams of protein as a percentage of the RDA

| | APP | DFH whey | JF whey | GNC whey | Whey Isolate | Whey concentrate | JF soy | NB soy |
|---|---|---|---|---|---|---|---|---|
| Calcium | 55% | 12.5% | 9.0% | | 8.3% | 18.8% | 2.9% | 5.0% |
| Iron | 18.1% | 4.2% | 1.8% | | | | 22.2% | 22.2% |
| Magnesium | 10% | | 3.5% | | | | | 2.8% |
| Zinc | 9.8% | | | | | | | 6.7% |
| Sodium | 2.1% | 2.0% | 1.7% | 2.6% | 2% | 2.3% | | 0.6% |
| Potassium | 4.6% | 4.6% | 3.7% | 5.7% | 8.7% | 3.7% | 10.6% | 12.9% |
| Phosphorus | 18.4% | 21.3% | 8.9% | | | | | 29.3%, |

1. A composition comprising a protein population, wherein greater than 30% of proteins in said protein population comprise:
a) aldehydes;
b) amides;
c) triazenes;
d) carboxylic acids;
e) alkanes or any combination thereof.

2. The composition of claim 1, wherein greater than 50% of proteins in said protein population have a molecular weight of less than 6000 Daltons.

3. The composition of claim 1, wherein greater than 60% of proteins in said protein population have a molecular weight of less than 6000 Daltons.

4. The composition of claim 1, wherein greater than 30% of proteins in said protein population have a molecular weight of less than 6000 Daltons.

5. (canceled)

6. (canceled)

7. The composition of claim 1, wherein said protein population is water insoluble.

8. The composition of claim 1, wherein said protein population is non-hygroscopic.

9. The composition of claim 1, wherein a majority of proteins in said protein population are non-hydrolyzed proteins.

10. (canceled)

11. The composition of claim 1, wherein said protein population comprises one or more proteins with an aldehyde group.

12. (canceled)

13. (canceled)

14. The composition of claim 1, wherein said protein population comprises one or more proteins with a triazene group.

15. (canceled)

16. (canceled)

17. The composition of claim 1, wherein said protein population comprises one or more proteins with a carboxylic acid group.

18. (canceled)

19. (canceled)

20. The composition of claim 1, wherein said protein population comprises one or more proteins with an alkene group.

21. (canceled)

22. (canceled)

23. (canceled)

24. The composition of claim 1, wherein said protein population comprises one or more proteins with an amide group.

25. (canceled)

26. (canceled)

27. The composition of claim 1, wherein said protein population comprises one or more proteins with an alkane group.

28. (canceled)

29. (canceled)

30. The composition of claim 1 wherein at least 10% of proteins in said protein population are bioactive proteins.

31. (canceled)

32. (canceled)

33. (canceled)

34. (canceled)

35. The composition of claim 1 wherein said protein population is an angiotensin-converting-enzyme inhibitor.

36. The composition of claim 1, wherein greater than 30% of proteins in said protein population have from 50 to 60 amino acids.

37. The composition of claim 36, wherein greater than 50% of proteins in said protein population have from 50 to 60 amino acids.

38. The composition of claim 36, wherein greater than 90% of proteins in said protein population have from 50 to 60 amino acids.

39. The composition of claim 1, wherein said protein population is derived from an animal.

40. The composition of claim 39, wherein said animal is a fish.

41.-48. (canceled)
